(19) United States
(12) Patent Application Publication
(10) Pub. No.: US 2006/0293339 A1
(43) Pub. Date: Dec. 28, 2006

(54) Biaryl substituted 6-membered heterocycles as sodium channel blockers

(76) Inventors: Prasun K. Chakravarty, Edison, NJ (US); Michael H. Fisher, Ringoes, NJ (US); William H. Parsons, Belle Mead, NJ (US); Jun Liang, Eatontown, NJ (US); Bishan Zhou, Edison, NJ (US)

Correspondence Address: MERCK AND CO., INC, P O BOX 2000, RAHWAY, NJ, 07065-0907, US

(21) Appl. No.: 10/550,641

(22) PCT Filed: Mar. 19, 2004
(86) PCT No.: PCT/US04/08532

§ 371 Date: Sep. 23, 2005

Related U.S. Application Data
(60) Provisional application No. 60/456,312, filed on Mar. 24, 2003.

(51) Int. Cl. A61K 31/506; A61K 31/4965; A61K 31/497; A61K 31/455; A61K 31/4439; C07D 403/02; A61K 31/5377; C07D 413/02

(52) U.S. Cl. 514/255.05; 544/122; 544/124; 544/295; 544/296; 544/329; 544/331; 546/315; 544/405; 544/406; 514/255.06; 514/275; 514/355; 514/341

(57) ABSTRACT

Biaryl substituted pyridine, pyrimidine and pyrazine compounds are sodium channel blockers useful for the treatment of pain. Pharmaceutical compositions comprise an effective amount of the instant compounds, either alone, or in combination with one or more therapeutically active compounds, and a pharmaceutically acceptable carrier. Methods of treating conditions associated with, or caused by, sodium channel activity, including, for example, acute pain, chronic pain, visceral pain, inflammatory pain, neuropathic pain, epilepsy, irritable bowel syndrome, depression, anxiety, multiple sclerosis, and bipolar disorder, comprise administering an effective amount of the present compounds, either alone, or in combination with one or more other therapeutically active compounds.

### FIELD OF THE INVENTION

The present invention is directed to a series of biaryl substituted 6-membered heterocyclic compounds. In particular, this invention is directed to biaryl substituted 6-membered pyridine, pyrimidine and pyrazine compounds that are sodium channel blockers useful for the treatment of chronic and neuropathic pain. The compounds of the present invention are also useful for the treatment of other conditions, including, for example, central nervous system (CNS) disorders such as epilepsy, manic depression, bipolar disorder, anxiety, depression and diabetic neuropathy.

### BACKGROUND OF THE INVENTION

Voltage-gated ion channels allow electrically excitable cells to generate and propagate action potentials and therefore are crucial for nerve and muscle function. Sodium channels play a special role by mediating rapid depolarization, which constitutes the rising phase of the action potential and in turn activates voltage-gated calcium and potassium channels. Voltage-gated sodium channels represent a multigene family. Nine sodium channel subtypes have been cloned and functionally expressed to date. [Clare, J. J., Tate, S. N., Nobbs, M. & Romanos, M. A. Voltage-gated sodium channels as therapeutic targets. Drug Discovery Today 5, 506-520 (2000)]. They are differentially expressed throughout muscle and nerve tissues and show distinct biophysical properties. All voltage-gated sodium channels are characterized by a high degree of selectivity for sodium over other ions and by their voltage-dependent gating. [Catterall, W. A. Structure and function of voltage-gated sodium and calcium channels. Current Opinion in Neurobiology 1, 5-13 (1991)]. At negative or hyperpolarized membrane potentials, sodium channels are closed. Following membrane depolarization, sodium channels open rapidly and then inactivate. Sodium channels only conduct currents in the open state and, once inactivated, have to return to the resting state, favored by membrane hyperpolarization, before they can reopen. Different sodium channel subtypes vary in the voltage range over which they activate and inactivate as well as in their activation and inactivation kinetics.

Sodium channels are the target of a diverse array of pharmacological agents, including neurotoxins, antiarrhythmics, anticonvulsants and local anesthetics. [Clare, J. J., Tate, S. N., Nobbs, M. & Romanos, M. A. Voltage-gated sodium channels as therapeutic targets. Drug Discovery Today 5, 506-520 (2000)]. Several regions in the sodium channel secondary structure are involved in interactions with these blockers and most are highly conserved. Indeed, most sodium channel blockers known to date interact with similar potency with all channel subtypes. Nevertheless, it has been possible to produce sodium channel blockers with therapeutic selectivity and a sufficient therapeutic window for the treatment of epilepsy (e.g. lamotrigine, phenytoin and carbamazepine) and certain cardiac arrhythmias (e.g. lignocaine, tocainide and mexiletine).

It is well known that the voltage-gated Na⁺ channels in nerves play a critical role in neuropathic pain. Injuries of the peripheral nervous system often result in neuropathic pain persisting long after the initial injury resolves. Examples of neuropathic pain include, but are not limited to, postherpetic neuralgia, trigeminal neuralgia, diabetic neuropathy, chronic lower back pain, phantom limb pain, pain resulting from cancer and chemotherapy, chronic pelvic pain, complex regional pain syndrome and related neuralgias. It has been shown in human patients as well as in animal models of neuropathic pain, that damage to primary afferent sensory neurons can lead to neuroma formation and spontaneous activity, as well as evoked activity in response to normally innocuous stimuli. [Carter, G. T. and B. S. Galer, Advances in the management of neuropathic pain. Physical Medicine and Rehabilitation Clinics of North America, 2001. 12(2): p. 447459]. The ectopic activity of normally silent sensory neurons is thought to contribute to the generation and maintenance of neuropathic pain. Neuropathic pain is generally assumed to be associated with an increase in sodium channel activity in the injured nerve. [Baker, M. D. and J. N. Wood, Involvement of Na channels in pain pathways. TRENDS in Pharmacological Sciences, 2001. 22(1): p. 27-31].

Indeed, in rat models of peripheral nerve injury, ectopic activity in the injured nerve corresponds to the behavioral signs of pain. In these models, intravenous application of the sodium channel blocker and local anesthetic lidocaine can suppress the ectopic activity and reverse the tactile allodynia at concentrations that do not affect general behavior and motor function. [Mao, J. and L. L. Chen, Systemic lidocaine for neuropathic pain relief. Pain, 2000. 87: p. 7-17). These effective concentrations were similar to concentrations shown to be clinically efficacious in humans. [Tanelian, D. L. and W. G. Brose, Neuropathic pain call be relieved by drugs that are use-dependent sodium channel blockers: lidocaine, carbamazepine and mexiletine. Anesthesiology, 1991. 74(5): p. 949-951). In a placebo-controlled study, continuous infusion of lidocaine caused reduced pain scores in patients with peripheral nerve injury, and in a separate study, intravenous lidocaine reduced pain intensity associated with postherpetic neuralgia (PHN). [Mao, J. and L. L. Chen, Systemic lidocaine for neuropathic pain relief. Pain, 2000. 87: p. 7-17. Anger, T., et al., Medicinal chemistry of neuronal voltage-gated sodium channel blockers. Journal of Medicinal Chemistry, 2001. 44(2): p. 115-137]. Lidoderm®, lidocaine applied in the form of a dermal patch, is currently the only FDA approved treatment for PHN. [Devers, A. and B. S. Galer, Topical lidocaiize patch relieves a variety of neuropathic pain conditions: an open-label study. Clinical Journal of Pain, 2000. 16(3): p. 205-208].

In addition to neuropathic pain, sodium channel blockers have clinical uses in the treatment of epilepsy and cardiac arrhythmias. Recent evidence from animal models suggests that sodium channel blockers may also be useful for neuroprotection under ischaemic conditions caused by stroke or neural trauma and in patients with multiple sclerosis (MS). [Clare, J. J. et. al. And Anger, T. et. al.].

International Patent Publication WO 00/57877 describes aryl substituted pyrazoles, imidazoles, oxazoles, thiazoles, and pyrroles and their uses as sodium channel blockers. International Patent Publication WO 01/68612 describes aryl substituted pyridines, pyrimidines, pyrazines and triazines and their uses as sodium channel blockers. International Patent Publication WO 99/32462 describes triazine compounds for the treatment for CNS disorders. However, there remains a need for novel compounds and compositions that therapeutically block neuronal sodium channels with less side effects and higher potency than currently known compounds.

### SUMMARY OF THE INVENTION

The present invention is directed to biaryl substituted 6-membered pyridine, pyrimidine and pyrazine compounds which are sodium channel blockers useful for the treatment of chronic and neuropathic pain. The compounds of the present invention are also useful for the treatment of other conditions, including CNS disorders such as anxiety, depression, epilepsy, manic depression and bipolar disorder. This invention provides pharmaceutical compositions comprising a compound of the present invention, either alone, or in combination with one or more therapeutically active compounds, and a pharmaceutically acceptable carrier.

This invention further comprises methods for the treatment of conditions associated with, or resulting from, sodium channel activity, such as acute pain, chronic pain, visceral pain, inflammatory pain, neuropathic pain and disorders of the CNS including, but not limited to, anxiety, depression, epilepsy, manic depression and bipolar disorder.

### DETAILED DESCRIPTION OF THE INVENTION

The compounds described in the present invention are represented by Formula (I) or (II): 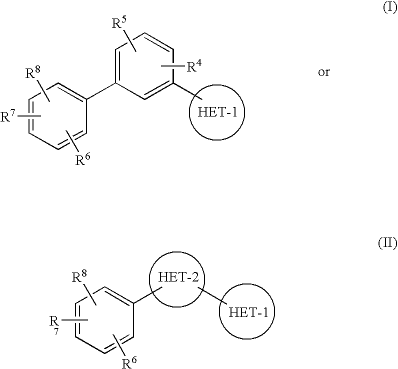
or a pharmaceutically acceptable salt thereof, wherein

HET-1 is one of the following heterocycles:

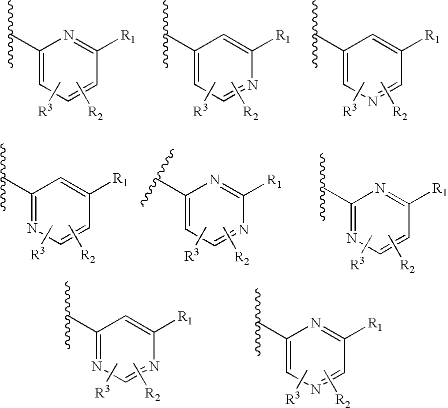

HET-2 is one of the following heterocycles: 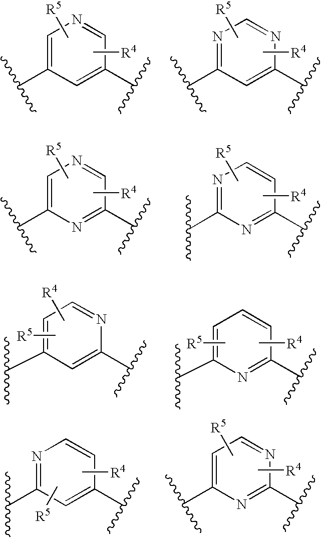
R¹ is
(a) H;
(b) C₁-C₆-alkyl, C₂-C₄-alkenyl, C₂-C₄-alkynyl, C₃-C₆-cycloalkyl, or C₁-C₄-alkyl-[C₃-C₆-cycloalkyl], any of which is optionally substituted with one or more of the following substituents: F, CF₃, OH, O—(C₁-C₄)alkyl, S(O)₀₋₂—(C₁-C₄)alkyl, O—CONR^{a}R^{b}, NR^{a}R^{b}, N(R^{a})CONR^{a}R^{b}, COO—(C₁-C₄)alkyl, COOH, CN, CONR^{a}R^{b}, SO₂NR^{a}R^{b}, N(R^{a})SO₂NR^{a}R^{b}, —C(═NH)NH₂, tetrazolyl, triazolyl, imidazolyl, oxazolyl, oxadiazolyl, isooxazolyl, thiazolyl, furyl, thienyl, pyrazolyl, pyrrolyl, pyridyl, pyrimidinyl, pyrazinyl, phenyl, piperidinyl, morpholinyl, pyrrolidinyl or piperazinyl;
(c) —O—C₁-C₆-alkyl, —O—C₃-C₆-cycloalkyl, —S—C₁-C₆-alkyl or —S—₃-₆-cycloalkyl, any of which is optionally substituted with one or more of the following substituents: F, CF₃, OH, O—(C₁-C₄)alkyl, S(O)₀₋₂—(C₁-C₄)alkyl, O—CONR^{a}R^{b}, NR^{a}R^{b}, N(R^{a})CONR^{a}R^{b}, COO—(C₁-C₄)alkyl, COOH, CN, CONR^{a}R^{b}, SO₂NR^{a}R^{b}, N(R^{a})SO₂NR^{a}R^{b}, —C(═NH)NH₂, tetrazolyl, triazolyl, imidazolyl, oxazolyl, oxadiazolyl, isooxazolyl, thiazolyl, furyl, thienyl, pyrazolyl, pyrrolyl, pyridyl, pyrimidinyl, pyrazinyl, phenyl, piperidinyl, morpholinyl, pyrrolidinyl or piperazinyl;
(d) —C₀-C₄-alkyl-C₁-C₄-perfluoroalkyl, or —CO—₄-alkyl-C₁-C₄-perfluoroalkyl;
(e) —OH;
(f) —O-aryl, or —C₁-C₄-alkyl-aryl, wherein aryl is phenyl, pyridyl, pyrimidinyl, furyl, thienyl, pyrrolyl, triazolyl, pyrazolyl, thiazolyl, isoxazolyl, oxazolyl, or oxadiazolyl, any aryl of which is optionally substituted with 1-3 substituents selected from i) F, Cl, Br, I, ii) —CN, iii) —NO₂, iv) —C(═O)(R^{a}), v) —OR^{a}, vi) —NR^{a}R^{b}, vii) —C₀₋₄alkyl-CO—OR^{a}, viii) -(C₀₋₄alkyl)—NH—CO—OR^{a}, ix) —CO—N(R^{a})(R^{b}), x) —S(O)₀₋₂R^{a}, xi) —SO₂N(R^{a})(R^{b}), xii) —NR^{a}SO₂R^{a}, xiii) —C₁₋₁₀alkyl, and xiv) —C₁₋₁₀alkyl, wherein one or more of the alkyl carbons can be replaced by a —NR^{a}—, —O—, —S(O)₁₋₂—, —O—C(O)—, —C(O)—, —C(O)—O—, —C(O)—N(R^{a})—, —N(R^{a})—C(O)—, —N(R^{a})—C(O)—N(R^{a})—, —C(O)—, —CH(OH)—, —C═C—, or —C≡C—;
(g) —OCON(R^{a})(R^{b}), or —OSO₂N(R^{a})(R^{b});
(h) —SH, or —SCON(R^{a})(R^{b});
(i) NO₂;
(j) NR^{a}R^{b}, —N(COR^{a})R^{b}, —N(SO₂R^{a})R^{b}, —N(R^{a})CON(R^{a})₂, —N(R^{a})CONH₂, —N(OR^{a})CONR^{a}R^{b}, —N(R^{a})CON(R^{a})₂, or —N(R^{a})SO₂N(R^{a})₂;
(k) —CH(OR^{a})R^{a}, —C(OR^{b})CF₃, —CH(NHR^{b})R^{a}, —C(═O)R^{a}, C(═O)CF₃, —SOCH₃, —SO₂CH₃, —N(R^{a})SO₂R^{a}, COOR^{a}, CN, CONR^{a}R^{b}, —COCONR^{a}R^{b}, —SO₂NR^{a}R^{b}, —CH₂O—SO₂NR^{a}R^{b}, SO₂N(R^{a})OR^{a}, —C(═NH)NH₂, —CR^{a}═N—OR^{a}, CH═CHCONR^{a}R^{b}, CONR^{a}, CONHR^{a};
(l) —CONR^{a}(CH₂)₀₋₂C(R^{a})(R^{b})(CH₂)₀₋₂CONR^{a}R^{b};
(m) tetrazolyl, tetrazolinonyl, triazolyl, triazolinonyl, imidazolyl, imidozolonyl, oxazoly], oxadiazolyl, isooxazolyl, thiazolyl, furyl, thienyl, pyrazolyl, pyrazolonyl, pyrrolyl, pyridyl, pyrimidinyl, pyrazinyl, or phenyl, any of which is optionally substituted with 1-3 substituents selected from i) F, Cl, Br, I, ii) —CN, iii) —NO₂, iv) —C(═O)R^{a}, v) C₁-C₆-alkyl, vi) —O—R^{a}, vii) —NR^{a}R^{b}, viii) —C₀-C₄-alkyl-CO—O R^{a}, ix) —(C₀-C₄-alkyl)—NH—CO—OR^{a}, x) —(C₀-C₄-alkyl)—CO—NR^{a}R^{b}, xi) —S(O)₀₋₂R^{a}, xii) —SO₂NR^{a}R^{b}, xiii) —NHSO₂R^{a}, xiv) —C₁-C₄-perfluoroalkyl, and xv) —O—C₁-C₄-perfluoroalkyl;
(n) —C(R^{a})═C(R^{b})—COOR^{a}, or C(R^{a})═C(R^{b})—CONR^{a}R^{b}; 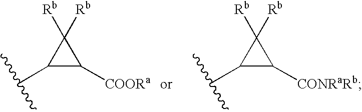
(p) piperidin-1-yl, morpholin-4-yl, pyrrolidin-1-yl, piperazin-1-yl or 4-susbstituted piperazin-1-yl, any of which is optionally substituted with 1-3 substituents selected from i) —CN, ii) —C(═O)(R^{a}), iii) C₁-C₆-alkyl, iv) —OR^{a}, v) —NR^{a}R^{b}, vi) —C₀-C₄-alkyl—CO—OR^{a}, vii) —(C₀-C₄-alkyl)—NH—CO—OR^{a}, viii) —(C₀-C₄-alkyl)—CON(R^{a})(R^{b}), ix) —SR^{a}, x) —S(O)₀₋₂R^{a}, xi) —SO₂N(R^{a})(R^{b}), xii) —NR^{a}SO₂R^{a} xiii) —C₁-C₄-perfluoroalkyl and xiv) —O—C₁-C₄-perfluoroalkyl;
R^{a} is
(a) H;
(b) C₁-C₄-alkyl, optionally substituted with one or more of the following substituents: F, CF₃, OH, O—(C₁-C₄)alkyl, S(O)₀₋₂—(C₁-C₄)alkyl, —OCONH₂, —OCONH(C₁-C₄alkyl), —OCON(C₁-C₄alkyl)(C₁-C₄alkyl), —OCONH(C₁-C₄alkyl-aryl), —OCON(C₁-C₄alkyl)(C₁-C₄alkyl-aryl), NH₂, NH(C₁-C₄alkyl), N(C₁-C₄alkyl)(C₁-C₄alkyl), NH(C₁-C₄alkyl-aryl), N(C₁-C₄alkyl)(C₁-C₄alkyl-aryl), NHCONH₂, NHCONH(C₁-C₄alkyl), NHCONH(C₁-C₄alkyl-aryl), —NHCON(C₁-C₄alkyl)(C₁-C₄alkyl), NHCON(C₁-C₄alkyl)(C₁-C₄alkyl-aryl), N(C₁-C₄alkyl)CON(C₁-C₄alkyl)(C₁-C₄alkyl), N(C₁-C₄alkyl)CON(C₁-C₄alkyl)(C₁-C₄alkyl-aryl), COO—(C₁-C₄-alkyl), COOH, CN, CONH₂, CONH(C₁-C₄alkyl), CON(C₁-C₄alkyl)(C₁-C₄alkyl), SO₂NH₂, SO₂NH(C₁-C₄alkyl), SO₂NH(C₁-C₄alkyl-aryl), SO₂N(C₁-C₄alkyl)(C₁-C₄alkyl), NHSO₂NH₂, —C(═NH)NH₂, tetrazolyl, triazolyl, imidazolyl, oxazolyl, oxadiazolyl, isooxazolyl, thiazolyl, furyl, thienyl, pyrazolyl, pyrrolyl, pyridyl, pyrimidinyl, pyrazinyl, phenyl, piperidinyl, morpholinyl, pyrrolidinyl or piperazinyl;
(c) C₀-C₄-alkyl-(C₁-C-₄)-perfluoroalkyl; or
(d) —C₁-C₄-alkyl-aryl, wherein aryl is phenyl, pyridyl, pyrirnidinyl, furyl, thienyl, pyrrolyl, triazolyl, pyrazolyl, thiazolyl, isoxazolyl, oxazolyl, or oxadiazolyl, any aryl of which is optionally substituted with 1-3 substituents selected from i) F, Cl, Br, I, ii) —CN, iii) —NO₂, iv) —C(═O)(C₁-C₄-alkyl), v) —O(C₁-C₄-alkyl), vi) —N(C₁-C₄-alkyl)(C₁-C₄-alkyl), vii) —C₁₋₁₀alkyl, and viii) —C₁₋₁₀alkyl, wherein one or more of the alkyl carbons can be replaced by a, —O—, —S(O)₁₋₂—, —O—C(O)—, —C(O)—O—, —C(O)—, —CH(OH)—, —C═C—, or —C≡C—;
R^{b} is
(a) H; or
(b) C₁-C₆-alkyl, optionally substituted with one or more of the following substituents: F, CF₃, OH, O—(C₁-C₄)alkyl, S(O)₀₋₂—(C₁-C₄)alkyl, —OCONH₂, —OCONH(C₁-C₄alkyl), NH₂, NH, NH(C₁-C₄alkyl), N(C₁-C₄alkyl), N(C₁-C₄alkyl)(C₁-C₄alkyl), NHCONH₂, NHCONH(C₁-C₄alkyl), —NHCON(C₁-C₄alkyl)(C₁-C₄alkyl), COO—(C₁-C₄-alkyl), COOH, CN, pyridyl, piperidinyl, pyrimidinyl, piperazinyl, CONH₂ or (C₁-C₄alkyl)CONH₂; or
R^{a} and R^{b}, together with the N to which they are attached, can form a 5- or 6-membered ring which optionally contains a heteroatom selected from N, O, and S, and wherein said ring is optionally substituted with 1-3 substituents selected from i) F, Cl, Br, I, ii) —CN, iii) —NO₂, iv) —C(═O)(R^{a}), v) —OR^{a}, vi) —NR^{a}R^{b}, vii) —C₀₋₄alkyl-CO—OR^{a}, viii) —(C₀₋₄alkyl)—NH—CO—OR^{a}, ix) —(C₀₋₄alkyl)—CO—N(R^{a})(R^{b}), x) —S(O)₀₋₂R^{a}, xi) —SO₂N(R^{a})(R^{b}), xii) —NR^{a}SO²R^{a}, xiii) —C₁₋₁₀alkyl, and xiv) —O—;
R² and R³ each independently is:
(a) H;
(b) —C₁-C₄-alkyl, or —O—C₁-C₄-alkyl;
(c) —C₀-C₄-alkyl—C₁-C₄-perfluoroalkyl, or —O—C₀-C₄-alkyl-C₁-C₄-perfluoroalkyl; or
(d) CN, N R^{a} R^{b}, NO₂, F, Cl, Br, I, OH, OCONR^{a} R^{b}, O(C₁-C₄-alkyl)CONR^{a} R^{b}, —OSO₂NR^{a} R^{b}, COOR^{a}, N(R^{a})COR^{a}, or CONR^{a} R^{b};
R⁴ and R⁵ each independently is:
(a) H;
(b) —C₁-C₆-alkyl, —C₂-C₆-alkenyl, —C₂-C₆-alkynyl or —C₃-C₆-cycloalkyl, any of which is optionally substituted with one or more of the following substituents: F, CF₃, —O—(C₁-C₄)alkyl, CN, —N(R^{a})(R^{b}), —N(R^{a})CO—(C₁-C₄)alkyl, COOR^{b}, CON(R^{a})(R^{b}) or phenyl;
(c) —O—C₀-C₆-alkyl, —O-aryl, or —O—C₁-C₄-alkyl-aryl, wherein aryl is phenyl, pyridyl, pyrimidinyl, furyl, thienyl, pyrrolyl, triazolyl, pyrazolyl, thiazolyl, isoxazolyl, oxazolyl, or oxadiazolyl, any aryl of which is optionally substituted with 1-3 substituents selected from i) F, Cl, Br, I, ii) —CN, iii) —NO₂, iv) —C(═O)(R^{a}), v) —OR^{a}, vi) —NR^{a}R^{b}, vii) —C₀₋₄alkyl-CO—OR^{a}, viii) —(C₀₋₄alkyl)—NH—CO—OR^{a}, ix) —(C₀₋₄alkyl)—CO—N(R^{a})(R^{b}), x) —S(O₀₋₂R^{a}, xi) —SO₂N(R^{a})(R^{b}), xii) —NR^{a}SO₂R^{a}, xiii) —C₁₋₁₀alkyl, and xiv) —C₁₋₁₀alkyl, wherein one or more of the alkyl carbons can be replaced by a —NR^{a}—, —O—, —S(O)₁₋₂—, —O—C(O)—, —C(O)—O—, —C(O)—N(R^{a})—, —N(R^{a})—C(O)—, —N(R^{a})—C(O)—N(R^{a})—, —C(O)—, —CH(OH)—, —C═C—, or —C≡C—;
(d) —C₀-C₄-alkyl-C₁-C₄-perfluoroalkyl, or —O—C₀-C₄-alkyl-C₁-C₄-perfluoroalkyl; or
(e) CN, NH₂, NO₂, F, Cl, Br, I, OH, OCON(R^{a})(R^{b})O(C₁-C₄-alkyl)CONR^{a}R^{b}, —OSO₂N(R^{a})(R^{b}), COOR^{b}, CON(R^{a})(R^{b}), or aryl, wherein aryl is phenyl, pyridyl, pyrimidinyl, furyl, thienyl, pyrrolyl, triazolyl, pyrazolyl, thiazolyl, isoxazolyl, oxazolyl, or oxadiazolyl, any aryl of which is optionally substituted with 1-3 substituents selected from i) F, Cl, Br, I, ii) —CN, iii) —NO₂, iv) —C(═O)(R^{a}), v) —OR^{a}, vi) —NR^{a}R^{b}, vii) —C₀₋₄alkyl-CO—OR^{a}, viii) —(C₀₋₄alkyl)—NH—CO—OR^{a}, ix) —(C₀₋₄alkyl)—CO—N(R^{a})(R^{b}), x) —S(O)₀₋₂R^{a}, xi) —SO₂N(R^{a})(R^{b}), xii) —NR^{a}SO₂R^{a}, xiii) —C₁₋₁₀alkyl, and xiv) —C₁₋₁₀alkyl, wherein one or more of the alkyl carbons can be replaced by a —NR^{a}—, —O—, —S(O)₁₋₂—, —O—C(O)—, —C(O)—O—, —C(O)—N(R^{a})—, —N(R^{a})—C(O)—, —N(R^{a})—C(O)—N(R^{a})—, —C(O)—, —CH(OH)—, —C═C—, or —C≡C; and
R⁶, R⁷ and R⁸ each independently is:
(a) H;
(b) C₁-C₆-alkyl, C₂-C₄-alkenyl, C₂-C₄-alkynyl or C₃-C6-cycloalkyl, any of which is optionally substituted with one or more of the following substituents: F, CF₃, OH, O—(C₁-C₄)alkyl, OCON(R^{a})(R^{b}), NR^{a}R^{b}, COOR^{a}, CN, CONR^{a}R^{b}, N(R^{a})CONR^{a}R^{b}, N(R^{a})SO₂NR^{a}R^{b}, SO₂NR^{a}R^{b}, S(O)₀₋₂(C₁-C₄-alkyl), —C(═NH)NH₂, tetrazolyl, triazolyl, imidazolyl, oxazolyl, oxadiazolyl, isooxazolyl, thiazolyl, furyl, thienyl, pyrazolyl, pyrrolyl, pyridyl, pyrimidinyl, pyrazinyl, phenyl, piperidinyl, morpholinyl, pyrrolidinyl, or piperazinyl;
(c) —O—C₁-C₆-alkyl, —O—C₃-C₆-cycloalkyl, —S—C₁-C₆-alkyl or —S—C₃-C₆-cycloalkyl, any of which is optionally substituted with one or more of the following substituents: F, CF₃, OH, O—(C₁-C₄)alkyl, NH₂, NH(C₁-C₄-alkyl), N(C₁-C₄-alkyl)₂, COOH, CN, CONH₂, CONH(C₁-C₄-alkyl), CONH(C₁-C₄-alkyl)₂, SO₂NH₂, SO₂NH(C₁-C₄-alkyl), tetrazolyl, triazolyl, imidazolyl, oxazolyl, oxadiazolyl, isooxazolyl, thiazolyl, furyl, thienyl, pyrazolyl, pyrrolyl, pyridyl, pyrimidinyl, pyrazinyl, phenyl, piperidinyl, morpholinyl, pyrrolidinyl, or piperazinyl;
(d) —C₀-C₄-alkyl-C₁-C₄-perfluoroalkyl, or —O—C_{0-C}₄-alkyl-C₁-C₄-perfluoroalkyl;
(e) —O-aryl, or —O—C₁-C₄-alkyl-aryl, wherein aryl is phenyl, pyridyl, pyrimidinyl, furyl, thienyl, pyrrolyl, triazolyl, pyrazolyl, thiazolyl, isoxazolyl, oxazolyl, or oxadiazolyl, any aryl of which is optionally substituted with 1-3 substituents selected from i) F, Cl, Br, I, ii) —CN, iii) —NO₂, iv) —C(═O)(R^{a}), v) —OR^{a}, vi) —NR^{a}R^{b}, vii) —C₀₋₄alkyl-CO—OR^{a}, viii) —(C₀₋₄alkyl)—NH—CO—OR^{a}, ix) —(C₀₋₄alkyl)—CO—N(R^{a})(R^{b}), x) —S(O)₀₋₂R^{a}, xi) —SO₂N(R^{a})(R^{b}), xii) —NR^{a}SO₂R^{a}, xiii) —C₁₋₁₀alkyl, and xiv) —C₁₋₁₀alkyl, wherein one or more of the alkyl carbons can be replaced by a —NR^{a}—, —O—, —S(O)₁₋₂—, —O—C(O)—, —C(O)—O—, —C(O)—N(R^{a})—, —N(R^{a})—C(O)—, —N(R^{a})—C(O)—N(R^{a})—, —C(O)—, —CH(OH)—, —C═C—, or —C≡C;
(f) CN, N(R^{a})(R^{b}), NO₂, F, Cl, Br, I, —OR^{a}, —SR^{a}, —OCON(R^{a})(R^{b}), —OSO₂N(R^{a})(R^{b}), COOR^{b}, CON(R^{a})(R^{b}), —N(R^{a})CON(R^{a}(R^{b}), —N(R^{a})SO₂N(R^{a})(R^{b}), —C(OR^{b})R^{a}, —C(OR^{a})CF₃, —C(NHR^{a})CF₃, —C(═O)R^{a}, C(═O)CF₃, —SOCH₃, —SO₂CH₃, —NHSO₂(C₁₋₆-alkyl), —NHSO₂-aryl, SO₂N(R^{a})(R^{b}), —CH₂OSO₂N(R^{a})(R^{b}), SO₂N(R^{b})—OR^{a}, —C(═NH)NH₂, —CR^{a}═N—ORₐ, CH═CH or aryl, wherein aryl is phenyl, pyridyl, pyrimidinyl, furyl, thienyl, pyrrolyl, triazolyl, pyrazolyl, thiazolyl, isoxazolyl, oxazolyl, or oxadiazolyl, any aryl of which is optionally substituted with 1-3 substituents selected from i) F, Cl, Br, I, ii) —CN, iii) —NO₂, iv) —C(═O)(R^{a}), v) —OR^{a}, vi) —NR^{a}R^{b}, vii) —C₀₋₄alkyl-CO—OR^{a}, viii) —(C₀₋₄alkyl)—NH—CO—OR^{a}, ix) —(C₀₋₄alkyl)—CO—N(R^{a})(R^{b}), x) —S(O)₀₋₂R^{a}, xi) —SO₂N(R^{a}(R^{b}), xii) —NR^{a}SO₂R^{a}, xiii) —C₁₋₁₀alkyl, and xiv) —C₁₋₁₀alkyl, wherein one or more of the alkyl carbons can be replaced by a —NR^{a}—, —O—, —S(O)₁₋₂—, —O—C(O)—, —C(O)—O—, —C(O)—N(R^{a})—, —N(R^{a})—C(O)—, —N(R^{a})—C(O)—N(R^{a})—, —C(O)—, —CH(OH)—, —C═C—, or —C≡C; or

when R⁶ and R⁷ are present on adjacent carbon atoms, R⁶ and R⁷, together with the benzene ring to which they are attached, can form a bicyclic aromatic ring selected from naphthyl, indolyl, quinolinyl, isoquinolinyl, quinoxalinyl. benzofuryl, benzothienyl, benzoxazolyl, benzothiazolyl, and benzimidazolyl, any of which is optionally substituted with 1-4 independent substituents selected from i) halogen, ii) —CN, iii) —NO₂, iv) —CHO, v) —O—C₁₋₄alkyl, vi) —N(C₀₋₄alkyl)(C₀₋₄alkyl), vii) —C₀₋₄alkyl-CO—O(C₀₋₄alkyl), viii) —(C₀₋₄alkyl)—NH—CO—O(C₀₋₄alkyl), ix) —(C₀₋₄alkyl)—CO—N(C₀₋₄alkyl)(C₀₋₄alkyl), x) —S(C₀₋₄alkyl), xi) —S(O)(C₁₋₄alkyl), xii) —SO₂(C₀₋₄alkyl), xiii) —SO₂N(C₀₋₄alkyl)(C₀₋₄alkyl), xiv) —NHSO₂(C₀₋₄alkyl)(C₀₋₄alkyl), xv) —C₁₋₁₀alkyl and xvi) —C₁₋₁₀alkyl in which one or more of the carbons can be replaced by a —N(C₀₋₆alkyl)—, —O—, —S(O)₁₋₂—, —O—C(O)—, —C(O)—O—, —C(O)—N(C₀₋₆alkyl)—, —N(C₀₋₆alkyl)—C(O)—, —N(C₀₋₆alkyl)—C(O)—N(C₀₋₆alkyl)—, —C(O)—, —CH(OH), —C═C—, or —C≡C—.

In one aspect, the present invention provides a compound described by the chemical Formula (I), or a pharmaceutically acceptable salt thereof.

In an embodiment of this one aspect, the present invention provides a compound described by the chemical Formula (I), or a pharmaceutically acceptable salt thereof, wherein

HET-1 is

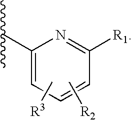

In another embodiment of this one aspect, the present invention provides a compound described by the chemical Formula (I), or a pharmaceutically acceptable salt thereof, wherein

HET-1 is

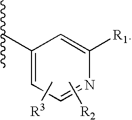

In a further embodiment of this one aspect, the present invention provides a compound described by the chemical Formula (I), or a pharmaceutically acceptable salt thereof, wherein

HET-1 is

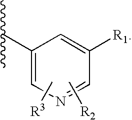

In yet another embodiment of this one aspect, the present invention provides a compound described by the chemical Formula (I), or a pharmaceutically acceptable salt thereof, wherein

HET-1 is

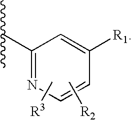

In a still further embodiment of this one aspect, the present invention provides a compound described by the chemical Formula (I), or a pharmaceutically acceptable salt thereof, wherein

HET-1 is

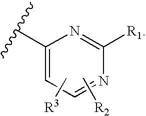

In a still other embodiment of this one aspect, the present invention provides a compound described by the chemical Formula (I), or a pharmaceutically acceptable salt thereof, wherein

HET-1 is

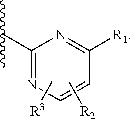

In yet still another embodiment of this one aspect, the present invention provides a compound described by the chemical Formula (I), or a pharmaceutically acceptable salt thereof, wherein

HET-1 is

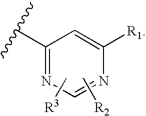

In a yet further embodiment of this one aspect, the present invention provides a compound described by the chemical Formula (I), or a pharmaceutically acceptable salt thereof, wherein

HET-1 is

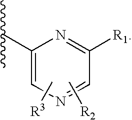

In a yet still further embodiment of this one aspect, the present invention provides a compound described by the chemical Formula (I), or a pharmaceutically acceptable salt thereof, wherein

R⁶ is other than H and is attached at the ortho position.

In a second aspect, the present invention provides a compound described by the chemical Formula (II), or a pharmaceutically acceptable salt thereof.

In an embodiment of this second aspect, the present invention provides a compound described by the chemical Formula (II), or a pharmaceutically acceptable salt thereof, wherein

HET-2 is

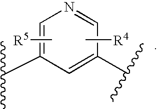

In another embodiment of this second aspect, the present invention provides a compound described by the chemical Formula (II), or a pharmaceutically acceptable salt thereof, wherein

HET-2 is

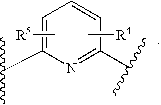

In a further embodiment of this second aspect, the present invention provides a compound described by the chemical Formula (II), or a pharmaceutically acceptable salt thereof, wherein

HET-2 is

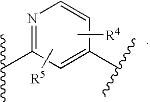

In a still further embodiment of this second aspect, the present invention provides a compound described by the chemical Formula (II), or a pharmaceutically acceptable salt thereof, wherein

HET-1 is

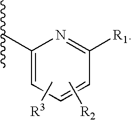

In yet another embodiment of this second aspect, the present invention provides a compound represented by the Formula (II), or a pharmaceutically acceptable salt thereof, wherein

HET-1 is

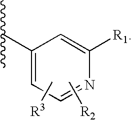

In an other embodiment of this second aspect, the present invention provides a compound represented by the Formula (I), or a pharmaceutically acceptable salt thereof, wherein

HET-1 is

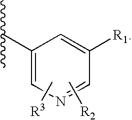

In a still other embodiment of this second aspect, the present invention provides a compound represented by the Formula (II), or a pharmaceutically acceptable salt thereof, wherein

HET-1 is

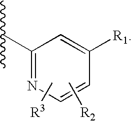

In yet still another embodiment of this second aspect, the present invention provides a compound represented by the Formula (II), or a pharmaceutically acceptable salt thereof, wherein

HET-1 is

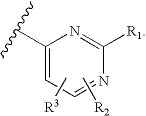

In a yet further embodiment of this second aspect, the present invention provides a compound represented by the Formula (II), or a pharmaceutically acceptable salt thereof, wherein

HET-1 is

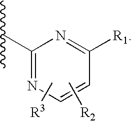

In a yet still further embodiment of this second aspect, the present invention provides a compound represented by the Formula (I), or a pharmaceutically acceptable salt thereof, wherein

HET-1 is

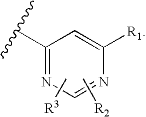

In an additional embodiment of this second aspect, the present invention provides a compound represented by the Formula (II), or a pharmaceutically acceptable salt thereof, wherein

HET-1 is

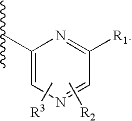

In a still additional embodiment of this second aspect, the present invention provides a compound represented by the Formula (II), or a pharmaceutically acceptable salt thereof, wherein

HET-2 is

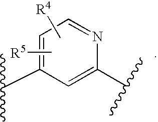

In a yet additional embodiment of this second aspect, the present invention provides a compound represented by the Formula (II), or a pharmaceutically acceptable salt thereof, wherein

HET-2 is

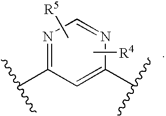

In a further additional embodiment of this second aspect, the present invention provides a compound represented by the Formula (II), or a pharmaceutically acceptable salt thereof, wherein

HET-2 is

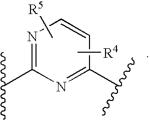

In a yet still other embodiment of this second aspect, the present invention provides a compound represented by the Formula (II), or a pharmaceutically acceptable salt thereof, wherein

HET-2 is

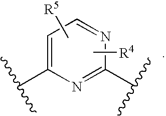

In a yet still another embodiment of this second aspect, the present invention provides a compound represented by the Formula (II), or a pharmaceutically acceptable salt thereof, wherein

HET-2 is

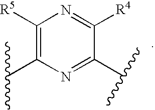

In a third aspect, the present invention provides a compound represented by the Formula (II), or a pharmaceutically acceptable salt thereof, wherein

HET-1 is 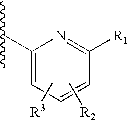
and

HET-2 is

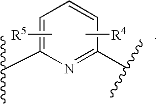

In a fourth aspect, the present invention provides a compound represented by the Formula (II), or a pharmaceutically acceptable salt thereof, wherein

HET-1 is 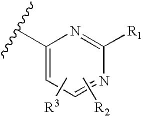
and

HET-2 is

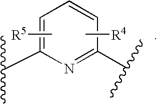

In a fifth aspect, the present invention provides a compound represented by the Formula (II), or a pharmaceutically acceptable salt thereof, wherein

HET-1 is 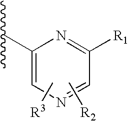
and

HET-2 is

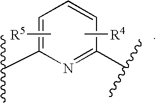

As used herein, “alkyl” as well as other groups having the prefix “alk” such as, for example, alkoxy, alkanoyl, alkenyl, and alkynyl means carbon chains which may be linear or branched or combinations thereof. Examples of alkyl groups include methyl, ethyl, propyl, isopropyl, butyl, sec- and tert-butyl, pentyl, hexyl, and heptyl. “Alkenyl,” “alkynyl” and other like terms include carbon chains containing at least one unsaturated C—C bond.

The term “cycloalkyl” means carbocycles containing no heteroatoms, and includes mono-, bi- and tricyclic saturated carbocycles, as well as fused ring systems. Such fused ring systems can include one ring that is partially or fully unsaturated such as a benzene ring to form fused ring systems such as benzofused carbocycles. Cycloalkyl includes such fused ring systems as spirofused ring systems. Examples of cycloalkyl include cyclopropyl, cyclobutyl, cyclopentyl, cyclohexyl, decahydronaphthalene, adamantane, indanyl, indenyl, fluorenyl, and 1,2,3,4-tetrahydronaphalene. Similarly, “cycloalkenyl” means carbocycles containing no heteroatoms and at least one non-aromatic C—C double bond, and include mono-, bi- and tricyclic partially saturated carbocycles, as well as benzofused cycloalkenes. Examples of cycloalkenyl include cyclohexenyl, and indenyl.

The term “aryl” includes, but is not limited to, an aromatic substituent that is a single ring or multiple rings fused together. When formed of multiple rings, at least one of the constituent rings is aromatic. The term “aryl”, unless specifically noted otherwise, also includes heteroaryls, and thus includes stable 5- to 7-membered monocyclic and stable 9- to 10-membered fused bicyclic heterocyclic ring systems that consist of carbon atoms and from one to four heteroatoms selected from the group consisting of N, O and S, wherein the nitrogen and sulfur heteroatoms may optionally be oxidized, and the nitrogen heteroatom may optionally be quaternized. Suitable aryl groups include phenyl, naphthyl, pyridyl, pyrimidinyl, furyl, thienyl, pyrrolyl, triazolyl, pyrazolyl, thiazolyl, isoxazolyl, oxazolyl, and oxadiazolyl.

The term “cycloalkyloxy,” unless specifically stated otherwise, includes a cycloalkyl group connected by a short C₁₋₂alkyl to the oxy connecting atom.

The term “C₀₋₆alkyl” includes alkyls containing 6, 5, 4, 3, 2, 1, or no carbon atoms. An alkyl with no carbon atoms is a hydrogen atom substituent when the alkyl is a terminal group and is a direct bond when the alkyl is a bridging group.

The term “hetero,” unless specifically stated otherwise, includes one or more O, S, or N atoms. For example, heterocycloalkyl and heteroaryl include ring systems that contain one or more O, S, or N atoms in the ring, including mixtures of such atoms. The hetero atoms replace ring carbon atoms. Thus, for example, a heterocycloC₅alkyl is a five-member ring containing from 4 to no carbon atoms. Examples of heteroaryls include pyridinyl, quinolinyl, isoquinolinyl, pyridazinyl, pyrimidinyl, pyrazinyl, quinoxalinyl, furyl, benzofuryl, dibenzofuryl, thienyl, benzthienyl, pyrrolyl, indolyl, pyrazolyl, indazolyl, oxazolyl, benzoxazolyl, isoxazolyl, thiazolyl, benzothiazolyl, isothiazolyl, imidazolyl, benzimidazolyl, oxadiazolyl, thiadiazolyl, triazolyl, and tetrazolyl. Examples of heterocycloalkyls include azetidinyl, pyrrolidinyl, piperidinyl, piperazinyl, morpholinyl, tetrahydrofuranyl, imidazolinyl, pyrolidin-2-one, piperidin-2-one, and thiomorpholinyl.

The term “heteroC₀₋₄alkyl” means a heteroalkyl containing 3, 2, 1, or no carbon atoms. However, at least one heteroatom must be present. Thus, as an example, a heteroC₀₋₄alkyl having no carbon atoms but one N atom would be a —NH— if a bridging group and a —NH₂ if a terminal group. Analogous bridging or terminal groups are clear for an O or S heteroatom.

The term “amine,” unless specifically stated otherwise, includes primary, secondary and tertiary amines.

The term “carbonyl,” unless specifically stated otherwise, includes a C₀₋₆alkyl substituent group when the carbonyl is terminal.

The term “halogen” includes fluorine, chlorine, bromine and iodine atoms.

The term “optionally substituted” is intended to include both substituted and unsubstituted. Thus, for example, optionally substituted aryl could represent a pentafluorophenyl or a phenyl ring. Further, optionally substituted multiple moieties such as, for example, alkylaryl are intended to mean that the alkyl and the aryl groups are optionally substituted. If only one of the multiple moieties is optionally substituted then it will be specifically recited such as “an alkylaryl, the aryl optionally substituted with halogen or hydroxyl.”

Compounds described herein may contain one or more double bonds and may thus give rise to cis/trans isomers as well as other conformational isomers. The present invention includes all such possible isomers as well as mixtures of such isomers unless specifically stated otherwise.

Compounds described herein can contain one or more asymmetric centers and may thus give rise to diastereoisomers and optical isomers. The present invention includes all such possible diastereoisomers as well as their racemic mixtures, their substantially pure resolved enantiomers, all possible geometric isomers, and pharmaceutically acceptable salts thereof. The above chemical Formulas are shown without a definitive stereochemistry at certain positions. The present invention includes all stereoisomers of the chemical Formulas and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolaamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, and tromethamine.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like.

The pharmaceutical compositions of the present invention comprise a compound represented by Formula I or II (or pharmaceutically acceptable salts thereof) as an active ingredient, a pharmaceutically acceptable carrier, and optionally one or more additional therapeutic agents or adjuvants. Such additional therapeutic agents can include, for example, i) opiate agonists or antagonists, ii) calcium channel antagonists, iii) 5HT receptor agonists or antagonists iv) sodium channel antagonists, v) NMDA receptor agonists or antagonists, vi) COX-2 selective inhibitors, vii) NKl antagonists, viii) non-steroidal anti-inflammatory drugs (“NSAID”), ix) selective serotonin reuptake inhibitors (“SSRI”) and/or selective serotonin and norepinephrine reuptake inhibitors (“SSNRI”), x) tricyclic antidepressant drugs, xi) norepinephrine modulators, xii) lithium, xiii) valproate, and xiv) neurontin (gabapentin). The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

The present compounds and compositions are useful for the treatment of chronic, visceral, inflammatory and neuropathic pain syndromes. They are useful for the treatment of pain resulting from traumatic nerve injury, nerve compression or entrapment, postherpetic neuralgia, trigeminal neuralgia, and diabetic neuropathy. The present compounds and compositions are also useful for the treatment of chronic lower back pain, phantom limb pain, chronic pelvic pain, neuroma pain, complex regional pain syndrome, chronic arthritic pain and related neuralgias, and pain associated with cancer, chemotherapy, HIV and HIV treatment-induced neuropathy. Compounds of this invention may also be utilized as local anesthetics. Compounds of this invention are useful for the treatment of irritable bowel syndrome and related disorders, as well as Crohns disease.

The instant compounds have clinical uses for the treatment of epilepsy and partial and generalized tonic seizures. They are also useful for neuroprotection under ischaemic conditions caused by stroke or neural trauma and for treating multiple sclerosis. The present compounds are useful for the treatment of tachy-arrhythmias. Additionally, the instant compounds are useful for the treatment of neuropsychiatric disorders, including mood disorders, such as depression or more particularly depressive disorders, for example, single episodic or recurrent major depressive disorders and dysthyric disorders, or bipolar disorders, for example, bipolar I disorder, bipolar II disorder and cyclothymic disorder; anxiety disorders, such as panic disorder with or without agoraphobia, agoraphobia without history of panic disorder, specific phobias, for example, specific animal phobias, social phobias, obsessive-compulsive disorder, stress disorders including post-traumatic stress disorder and acute stress disorder, and generalised anxiety disorders;

It will be appreciated that for the treatment of depression or anxiety, a compound of the present invention may be used in conjunction with other anti-depressant or anti-anxiety agents, such as norepinephrine reuptake inhibitors, selective serotonin reuptake inhibitors (SSRIs), monoamine oxidase inhibitors (MAOIs), reversible inhibitors of monoamine oxidase (RMAs), serotonin and noradrenaline reuptake inhibitors (SNRIs), α-adrenoreceptor antagonists, atypical anti-depressants, benzodiazepines, 5-HT_{1A} agonists or antagonists, especially 5-HT_{1A} partial agonists, neurokinin-1 receptor antagonists, corticotropin releasing factor (CRF) antagonists, and pharmaceutically acceptable salts thereof.

Further, it is understood that compounds of this invention can be administered at prophylactically effective dosage levels to prevent the above-recited conditions and disorders, as well as to prevent other conditions and disorders associated with sodium channel activity.

Creams, ointments, jellies, solutions, or suspensions containing the instant compounds can be employed for topical use. Mouth washes and gargles are included within the cope of topical use for the purposes of this invention.

Dosage levels from about 0.01 mg/kg to about 140 mg/kg of body weight per day are useful in the treatment of inflammatory and neuropathic pain, or alternatively about 0.5 mg to about 7 g per patient per day. For example, inflammatory pain may be effectively treated by the administration of from about 0.01 mg to about 75 mg of the compound per kilogram of body weight per day, or alternatively about 0.5 mg to about 3.5 g per patient per day. Neuropathic pain may be effectively treated by the administration of from about 0.01 mg to about 125 mg of the compound per kilogram of body weight per day, or alternatively about 0.5 mg to about 5.5 g per patient per day.

The amount of active ingredient that may be combined with the carrier materials to produce a single dosage form will vary depending upon the host treated and the particular mode of administration. For example, a formulation intended for the oral administration to humans may conveniently contain from about 0.5 mg to about 5 g of active agent, compounded with an appropriate and convenient amount of carrier material which may vary from about 5 to about 95 percent of the total composition. Unit dosage forms will generally contain between from about 1 mg to about 1000 mg of the active ingredient, typically 25 mg, 50 mg, 100 mg, 200 mg, 300 mg, 400 mg, 500 mg, 600 mg, 800 mg or 1000 mg.

It is understood, however, that the specific dose level for any particular patient will depend upon a variety of factors. Such patient-related factors include the age, body weight, general health, sex, and diet of the patient. Other factors include the time and route of administration, rate of excretion, drug combination, and the severity of the particular disease undergoing therapy.

In practice, the compounds represented by Formula I or II, or pharmaceutically acceptable salts thereof, can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds represented by Formula I or II, or pharmaceutically acceptable salts thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt of Formula I or II. The compounds of Formula I or II, or pharmaceutically acceptable salts thereof, can also be included in pharmaceutical compositions in combination with one or more therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, and disintegrating agents can be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.1 mg to about 500 mg of the active ingredient and each cachet or capsule preferably containing from about 0.1 mg to about 500 mg of the active ingredient. Thus, a tablet, cachet, or capsule conveniently contains 0.1 mg, 1 mg, 5 mg, 25 mg, 50 mg, 100 mg, 200 mg, 300 mg, 400 mg, or 500 mg of the active ingredient taken one or two tablets, cachets, or capsules, once, twice, or three times daily.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage, and thus should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, and dusting powder. Further, the compositions can be in a form suitable for use in transdermal devices. These formulations may be prepared, utilizing a compound represented by Formula I or II, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid, such as, for example, where the mixture forms unit dose suppositories. Suitable carriers include cocoa butter and other materials commonly used in the art. The suppositories may be conveniently formed by first admixing the composition with the softened or melted carrier(s) followed by chilling and shaping in moulds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, and preservatives (including anti-oxidants). Furthermore, other adjuvants can be included to render the formulation isotonic with the blood of the intended recipient. Compositions containing a compound described by Formula I or II, or pharmaceutically acceptable salts thereof, may also be prepared in powder or liquid concentrate form.

The compounds and pharmaceutical compositions of this invention have been found to block sodium channels. Accordingly, an aspect of the invention is the treatment in mammals of maladies that are amenable to amelioration through blockage of neuronal sodium channels, including, for example, acute pain, chronic pain, visceral pain, inflammatory pain, and neuropathic pain by administering an effective amount of a compound of this invention. The term “mammals” includes humans, as well as other animals, such as, for example, dogs, cats, horses, pigs, and cattle. Accordingly, it is understood that the treatment of mammals other than humans refers to the treatment of clinical afflictions in non-human mammals that correlate to the above recited afflictions.

Further, as described above, the instant compounds can be utilized in combination with one or more therapeutically active compounds. In particular, the inventive compounds can be advantageously used in combination with i) opiate agonists or antagonists, ii) calcium channel antagonists, iii) 5HT receptor agonists or antagonists iv) sodium channel antagonists, v) N-methyl-D-aspartate (NMDA) receptor agonists or antagonists, vi) COX-2 selective inhibitors, vii) neurokinin receptor 1 (NK1) antagonists, viii) non-steroidal anti-inflammatory drugs (NSAID), ix) selective serotonin reuptake inhibitors (SSRI) and/or selective serotonin and norepinephrine reuptake inhibitors (SSNRI), x) tricyclic antidepressant drugs, xi) norepinephrine modulators, xii) lithium, xiii) valproate, and xiv) neurontin (gabapentin).

The abbreviations used herein have the following tabulated meanings. Abbreviations not tabulated below have their meanings as commonly used unless specifically stated otherwise.

| | |
|---|---|
| Ac | Acetyl |
| AIBN | 2,2′-azobis(isobutyronitrile) |
| BINAP | 1,1′-bi-2-naphthol |
| Bn | Benzyl |
| CAMP | cyclic adenosine-3′,5′-monophosphate |
| DAST | (diethylamino)sulfur trifluoride |
| DEAD | diethyl azodicarboxylate |
| DBU | 1,8-diazabicyclo[5.4.0]undec-7-ene |
| DIBAL | diisobutylaluminum hydride |
| DMAP | 4-(dimethylamino)pyridine |
| DMF | N,N-dimethylformamide |
| Dppf | 1,1′-bis(diphenylphosphino)-ferrocene |
| EDCI | 1-(3-dimethylaminopropyl)-3-ethylcarbodiimide |
| | hydrochloride |
| Et₃N | Triethylamine |
| GST | glutathione transferase |
| HMDS | Hexamethyldisilazide |
| LDA | lithium diisopropylamide |
| m-CPBA | metachloroperbenzoic acid |
| MMPP | monoperoxyphthalic acid |
| MPPM | monoperoxyphthalic acid, magnesium salt 6H₂O |
| Ms | methanesulfonyl = mesyl = SO₂Me |
| Ms0 | methanesulfonate = mesylate |
| NBS | N-bromo succinimide |
| NSAID | non-steroidal anti-inflammatory drug |
| o-Tol | ortho-tolyl |
| OXONE ® | 2KHSO₅.KHSO₄.K₂SO₄ |
| PCC | pyridinium chlorochromate |
| Pd₂(dba)₃ | Bis(dibenzylideneacetone) palladium(0) |
| PDC | pyridinium dichromate |
| PDE | Phosphodiesterase |
| Ph | Phenyl |
| Phe | Benzenediyl |
| PMB | para-methoxybenzyl |
| Pye | Pyridinediyl |
| r.t. or RT | room temperature |
| Rac. | Racemic |
| SAM | aminosulfonyl or sulfonamide or SO₂NH₂ |
| SEM | 2-(trimethylsilyl)ethoxymethoxy |
| SPA | scintillation proximity assay |
| TBAF | tetra-n-butylammonium fluoride |
| Th | 2- or 3-thienyl |
| TFA | trifluoroacetic acid |
| TFAA | trifluoroacetic acid anhydride |
| THF | Tetrahydrofuran |
| Thi | Thiophenediyl |
| TLC | thin layer chromatography |
| TMS-CN | trimethylsilyl cyanide |
| TMSI | trimethylsilyl iodide |
| Tz | 1H (or 2H)-tetrazol-5-yl |
| XANTPHOS | 4,5-Bis-diphenylphosphanyl-9,9-dimethyl-9H- |
| | xanthene |
| C₃H₅ | Allyl |

ALKYL GROUP ABBREVIATIONS

| | |
|---|---|
| Me = | Methyl |
| Et = | ethyl |
| n-Pr = | normal propyl |
| i-Pr = | isopropyl |
| n-Bu = | normal butyl |
| i-Bu = | isobutyl |
| s-Bu = | secondary butyl |
| t-Bu = | tertiary butyl |
| c-Pr = | cyclopropyl |
| c-Bu = | cyclobutyl |
| c-Pen = | cyclopentyl |
| c-Hex = | cyclohexyl |

The following in vitro and in vivo assays were used in assessing the biological activity of the instant compounds.

Compound Evaluation (in vitro Assay):

The identification of inhibitors of the sodium channel is based on the ability of sodium channels to cause cell depolarization when sodium ions permeate through agonist-modified channels. In the absence of inhibitors, exposure of an agonist-modified channel to sodium ions will cause cell depolarization. Sodium channel inhibitors will prevent cell depolarization caused by sodium ion movement through agonist-modified sodium channels. Changes in membrane potential can be determined with voltage-sensitive fluorescence resonance energy transfer (FRET) dye pairs that use two components, a donor coumarin (CC₂DMPE) and an acceptor oxanol (DiSBAC₂(3)). Oxanol is a lipophilic anion and distributes across the membrane according to membrane potential. In the presence of a sodium channel agonist, but in the absence of sodium, the inside of the cell is negative with respect to the outside, oxanol is accumulated at the outer leaflet of the membrane and excitation of coumarin will cause FRET to occur. Addition of sodium will cause membrane depolarization leading to redistribution of oxanol to the inside of the cell, and, as a consequence, to a decrease in FRET. Thus, the ratio change (donor/acceptor) increases after membrane depolarization. In the presence of a sodium channel inhibitor, cell depolarization will not occur, and therefore the distribution of oxanol and FRET will remain unchanged.

Cells stably transfected with the PN1 sodium channel (BEK-PN1) were grown in polylysine-coated 96-well plates at a density of ca. 140,000 cells/well. The media was aspirated, and the cells were washed with PBS buffer, and incubated with 100 μL of 10 μM CC₂-DMPE in 0.02% pluronic acid. After incubation at 25° C. for 45 min, media was removed and cells were washed 2× with buffer. Cells were incubated with 100 μL of DiSBAC₂(3) in TMA buffer containing 20 μM veratridine, 20 nM brevetoxin-3, and test sample. After incubation at 25° C. for 45 min in the dark, plates were placed in the VIPR instrument, and the fluorescence emission of both CC₂-DMPE and DiSBAC₂(3) recorded for 10 s. At this point, 100 μL of saline buffer was added to the wells to determine the extent of sodium-dependent cell depolarization, and the fluorescence emission of both dyes recorded for an additional 20 s. The ratio CC₂-DMPE/DiSBAC₂(3), before addition of saline buffer equals 1. In the absence of inhibitors, the ratio after addition of saline buffer is >1.5. When the sodium channel has been completely inhibited by either a known standard or test compound, this ratio remains at 1. It is possible, therefore, to titrate the activity of a sodium channel inhibitor by monitoring the concentration-dependent change in fluorescence ratio.

Electrophysiological Assays (In Vitro Assays):

Cell preparation: A HBEK-293 cell line stably expressing the PN1 sodium channel subtype was established in-house. The cells were cultured in MEM growth media (Gibco) with 0.5 mg/mL G418, 50 units/mL Pen/Strep and 1 mL heat-inactivated fetal bovine serum at 37° C. and 10% CO₂. For electrophysiological recordings, cells were plated on 35 mm dishes coated with poly-D-lysine.

Whole-cell recordings: HEK-293 cells stably expressing the PN1 sodium channel subtype were examined by whole cell voltage clamp (Hamill et. al. Pfluegers Archives 391:85-100 (1981)) using an EPC-9 amplifier and Pulse software (BEKA Electronics, Lamprecht, Germany). Experiments were performed at room temperature. Electrodes were fire-polished to resistances of 2-4 MΩ. Voltage errors were minimized by series resistance compensation, and the capacitance artifact was canceled using the EPC-9's built-in circuitry. Data were acquired at 50 kHz and filtered at 7-10 kHz. The bath solution consisted of 40 mM NaCl, 120 mM NMDG Cl, 1 mM KCl, 2.7 mM CaCl₂, 0.5 mnM MgCl₂, 10 mM NMDG HEPES, pH 7.4, and the internal (pipet) solution contained 110 mM Cs-methanesulfonate, 5 mM NaCl, 20 mM CsCl, 10 mM CsF, 10 mM BAPTA (tetra Cs salt), 10 mM Cs HEPES, pH 7.4.

The following protocols were used to estimate the steady-state affinity of compounds for the resting and inactivated state of the channel (Kᵣ and Kᵢ, respectively):

1) 8 ms test-pulses to depolarizing voltages from −60 mV to +50 mV from a holding potential of −90 mV were used to construct current-voltage relationships (IV-curves). A voltage near the peak of the IV-curve (typically −10 or 0 mV) was used as the test-pulse voltage throughout the remainder of the experiment.

2) Steady-state inactivation (availability) curves were constructed by measuring the current activated during an 8 ms test-pulse following 10 s conditioning pulses to potentials ranging from −120 mV to −10 mV.

3) Compounds were applied at a holding potential at which 20-50% of the channels was inactivated and sodium channel blockage was monitored during 8ms test pulses at 2 s intervals.

4) After the compounds equilibrated, the voltage-dependence of steady-state inactivation in the presence of compound was determined according to protocol 2) above. Compounds that block the resting state of the channel decrease the current elicited during test-pulses from all holding potentials, whereas compounds that primarily block the inactivated state shift the mid-point of the steady-state inactivation curve. The maximum current at negative holding potentials (Iₘₐₓ) and the difference in the mid-points of the steady-state inactivation curves (□V) in control and in the presence of a compound were used to calculate Kᵣ and Kᵢ using the following equations:

$${K}_{r} = \frac{[ Drug ] * {I}_{Max , Drug}}{{I}_{Max , Control} - {I}_{Max , Drug}}$$

$${K}_{i} = \frac{[ Drug ]}{( 1 + \frac{[ Drug ]}{{K}_{r}} ) * {ⅇ}^{\frac{- Δ V}{k}} - 1}$$

In cases where the compound did not affect the resting state, Kᵢ was calculated using the following equation:

$${K}_{i} = \frac{[ Drug ]}{{ⅇ}^{\frac{- Δ V}{k}} - 1}$$

Rat Formalin Paw Test (in vivo Assay):

Compounds were assessed for their ability to inhibit the behavioral response evoked by a 50 μL injection of formalin (5%). A metal band was affixed to the left hind paw of male Sprague-Dawley rats (Charles River, 200-250 g) and each rat was conditioned to the band for 60 min within a plastic cylinder (15 cm diameter). Rats were dosed with either vehicle or a test compound either before (local) or after (systemic) formalin challenge. For local administration, compounds were prepared in a 1:4:5 vehicle of ethanol, PEG400 and saline (EPEGS) and injected subcutaneously into the dorsal surface of the left hind paw 5 min prior to formalin. For systemic administration, compounds were prepared in either a EPEGS vehicle or a Tween80 (10%)/sterile water (90%) vehicle and were injected i.v. (via the lateral tail vein 15 min after formalin) or p.o. (60 min before formalin). The number of flinches was counted continuously for 60 min using an automated nociception analyzer (UCSD Anesthesiology Research, San Diego, Calif.). Statistical significance was determined by comparing the total flinches detected in the early (0-10 min) and late (11-60 min) phase with an unpaired t-test.

In vivo Assay using Rat CFA Model:

Unilateral inflammation was induced with a 0.2 ml injection of complete Freund's adjuvant (CFA: Mycobacterium tuberculosis, Sigma; suspended in an oil/saline (1:1) emulsion; 0.5 mg Mycobacterium/mL) in the plantar surface of the left hindpaw. This dose of CFA produced significant hind paw swelling but the animals exhibited normal grooming behavior and weight gain over the course of the experiment. Mechanical hyperalgesia was assessed 3 days after tissue injury using a Randall-Selitto test. Repeated Measures ANOVA, followed by Dunnett's Post Hoc test.

SNL: Mechanical Allodynia (in vivo Assay):

Tactile allodynia was assessed with calibrated von Frey filaments using an up-down paradigm before and two weeks following nerve injury. Animals were placed in plastic ages with a wire mesh floor and allowed to acclimate for 15 min before each test session. To determine the 50% response threshold, the von Frey filaments (over a range of intensities from 0.4 to 28.8 g) were applied to the mid-plantar surface for 8 s, or until a withdrawal response occurred. Following a positive response, an incrementally weaker stimulus was tested. If there was no response to a stimulus, then an incrementally stronger stimulus was presented. After the initial threshold crossing, this procedure was repeated for four stimulus presentations per animal per test session. Mechanical sensitivity was assessed 1 and 2 hr post oral administration of the test compound.

The compounds described in this invention displayed sodium channel blocking activity of from about <0.1 μM to about <50 μM in the in vitro assays described above. It is advantageous that the compounds display sodium channel blocking activity of <5 μM in the in vitro assays. It is more advantageous that the compounds display sodium channel blocking activity of <1 μM in the in vitro assays. It is even more advantageous that the compounds display sodium channel blocking activity of <0.5 μM in the in vitro assays. It is still more advantageous that the compounds display sodium channel blocking activity of <0.1 μM in the in vitro assays.

The present compounds can be prepared according to the general schemes provided below as well as the procedures provided in the Examples:. The following Schemes and Examples further describe, but do not limit, the scope of the invention.

Unless specifically stated otherwise, the experimental procedures were performed under the following conditions: All operations were carried out at room or ambient temperature; that is, at a temperature in the range of 18-25° C. Evaporation of solvent was carried out using a rotary evaporator under reduced pressure (600-4000 pascals: 4.5-30 mm. Hg) with a bath temperature of up to 60° C. The course of reactions was followed by thin layer chromatography (TLC) and reaction times are given for illustration only. Melting points are uncorrected and ‘d’ indicates decomposition. The melting points given are those obtained for the materials prepared as described. Polymorphism may result in isolation of materials with different melting points in some preparations. The structure and purity of all final products were assured by at least one of the following techniques: TLC, mass spectrometry, nuclear magnetic resonance (NMR) spectrometry or microanalytical data. When given, yields are for illustration only. When given, NMR data is in the form of delta (δ) values for major diagnostic protons, given in parts per million (ppm) relative to tetramethylsilane (TMS) as internal standard, determined at 300 Mz, 400 MHz or 500 MHz using the indicated solvent. Conventional abbreviations used for signal shape are: s. singlet; d. doublet; t. triplet; m. multiplet; br. broad; etc. In addition, “Ar” signifies an aromatic signal. Chemical symbols have their usual meanings; the following abbreviations, are used: v (volume), w (weight), b.p. (boiling point), m.p. (melting point), L (liter(s)), mL (milliliters), g (gram(s)), mg (milligrams(s)), mol (moles), mmol (millimoles), eq (equivalent(s)).

### Methods of Synthesis

Compounds of the present invention can be prepared according to the following methods. The substituents are the same as in the above Formulas except where defined otherwise.

The novel compounds of the present invention can be readily synthesized using techniques known to those skilled in the art, such as those described, for example, in Advanced Organic Chemistry, March, 4^{th} Ed., John Wiley and Sons, New York, NY, 1992; Advanced Organic Chemistry, Carey and Sundberg, Vol. A and B, 3^{rd} Ed., Plenum Press, Inc., New York, N.Y., 1990; Protective groups in Organic Synthesis, Green and Wuts, 2^{nd} Ed., John Wiley and Sons, New York, N.Y., 1991; Comprehensive Organic Transformations, Larock, VCH Publishers, Inc., New York, N.Y., 1988; Handbook of Heterocyclic Chemistry, Katritzky and Pozharskii, 2^{nd} Ed., Pergamon, New York, N.Y., 2000 and references cited therein. The starting materials for the present compounds may be prepared using standard synthetic transformations of chemical precursors that are readily available from commercial sources such as Aldrich Chemical Co. (Milwaukee, Wis.); Sigma Chemical Co. (St. Louis, Mo.); Lancaster Synthesis (Windham, N.H.); Ryan Scientific (Columbia, S.C.); Maybridge (Cornwall, UK); Matrix Scientific (Columbia, S. C.); Arcos, (Pittsburgh, Pa.) and Trans World Chemicals (Rockville, Md.).

The procedures described herein for synthesizing the compounds may include one or more steps of protecting group manipulations and various purification steps, such as, recrystallization, distillation, column chromatography, flash chromatography, thin-layer chromatography (TLC), radial chromatography and high-pressure chromatography (HPLC). The products can be characterized using various techniques well known in chemical arts, such as, proton and carbon-13 nuclear magnetic resonance (¹H and ¹³C NMR), infrared and ultraviolet spectroscopy (R and UV), X-ray crystallography, elemental analysis and BPLC and mass spectrometry (LC-MS). Methods of protecting group manipulation, purification, structure identification and quantification are well known to one skilled in the art of chemical synthesis.

Pyridine compounds of the present invention as represented by the formula shown immediately below can be prepared as outlined in SCHEME 1.

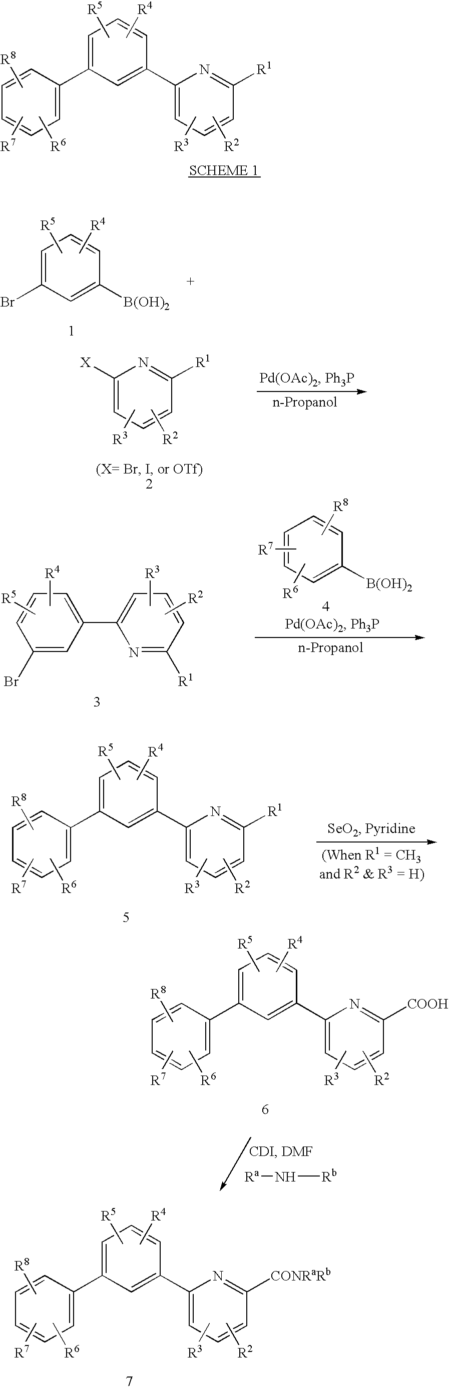

An appropriate bromo, iodo pyridine or trifluoromethanesulfonate (triflate) derivative 2 can be subjected to the Pd-catalyzed cross-coupling reaction (Suzuki reaction) [Huff, B. et al., Org. Synth. 75: 53-60 (1997); Goodson, F. E. et al. Org. Synth. 75: 61-68 (1997)) in the presence of an appropriately substituted aryl boronic acid 1 to provide 3, which can be then subjected to a second cycle of Suzuki reaction with 4 to give the biaryl pyridine compound 5. When R⁵ in 5 is a methyl group (R₅═Me), it can be oxidized under a mild condition as described to provide the carboxylic acid 6. The acid 6 can be converted to the amide 7 using an approprite amine R⁹—NH—R¹⁰ in the presence of an approprite carboxylic acid activating agent, such as carbonyl-di- imidazole (CDI). Alternatively, an appropriate ester or amide derivative of the commercially available 6-bromo-picolinic acid can be used in the synthesis of 7. The regioisomers of 7 also can be prepared by employing a similar sequence of reactions using appropriately substituted pyridine derivatives.

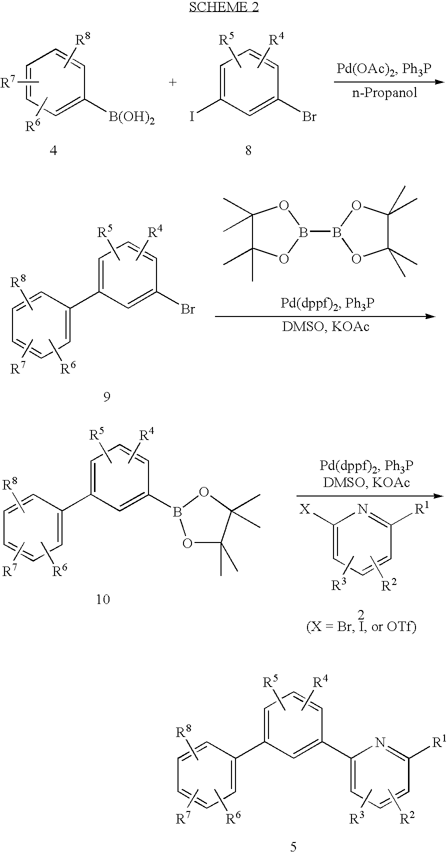

In an alternative approach to preparing pyridine compounds of the instant invention, the boronic acid 4 can be coupled with an appropriately substituted bromo, iodo or triflate derivative of 8 to provide the biphenyl 9, which can then be converted into the corresponding boronic acid ester 10 under the conditions described. The appropriate aryl or heteroaryl compound 2 can be then be coupled under Pd-catalyzed cross-coupling reaction condition to provide 5.

Compounds of the instant invention represented by the formula shown immediately below can be prepared as outlined in SCHEME 3.

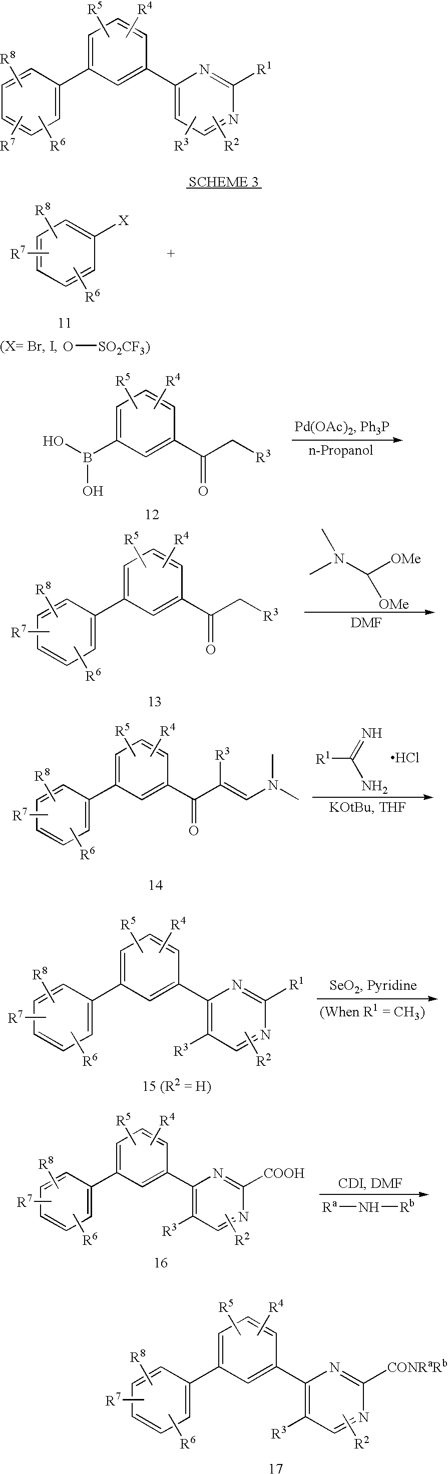

An appropriate aryl halide or aryl triflate 11 can be reacted with an appropriate boronic acid 12 under Pd-catalyzed cross-coupling reaction (Suzuki reaction) conditions to provide the ketone 13. The ketone can be converted to the intermediate 14, which can be then converted to the desired pyrimidine derivative 15 using the methods described by Domagala, J. M. et al. [J. Heterocyclic Chem. 26: 1147-1158 (1989)3 and Fischer, G. W. (J. Heterocyclic Chem. 26: 1147-1158 (1989)]. The methyl pyrimidine 15 (when R¹═CH₃) can be oxidized with SeO₂ using the conditions described by Sakamoto, T. et al, [Chem Pharm. Bull. 28: 571-577(1980)] to provide the corresponding carboxylic acid 16, which could then be elaborated into appropriate analogs including the amide 17 as described.

Alternatively, the biaryl pyrimidine 15 can also be synthesized by Pd-catalyzed cross-coupling reaction between the pyrimidine 20 and an appropriate aryl boronic acid 21 as outlined in SCHEME 4. A variety of aryl boronic acids are commercially available or these can be prepared conveniently from the corresponding aryl bromide or iodide by converting it to an organolithium derivative [Baldwin, J. E. et al. Tetrahedron Lett. 39: 707-710 (1998)) or a Grignard reagent followed by treatment with trialkylborate [Li, J. J. et al, J. Med. Chem, 38: 4570-4578(1995) and Piettre, S. R. et al. J. Med Chem. 40: 4208-4221 (1997)]. Aryl boronates can also be used as an alternative to aryl boronic acids in these Pd-catalyzed coupling reactions [Giroux, A. et. al., Tetrahedron Lett., 38: 3841(1997)]. The boronates can be easily prepared from the aryl bromides, iodides and trifluoromethane sulfonates using the method described by Murata, M. et. al. [J. Org. Chem. 65: 164-168 (2000)].

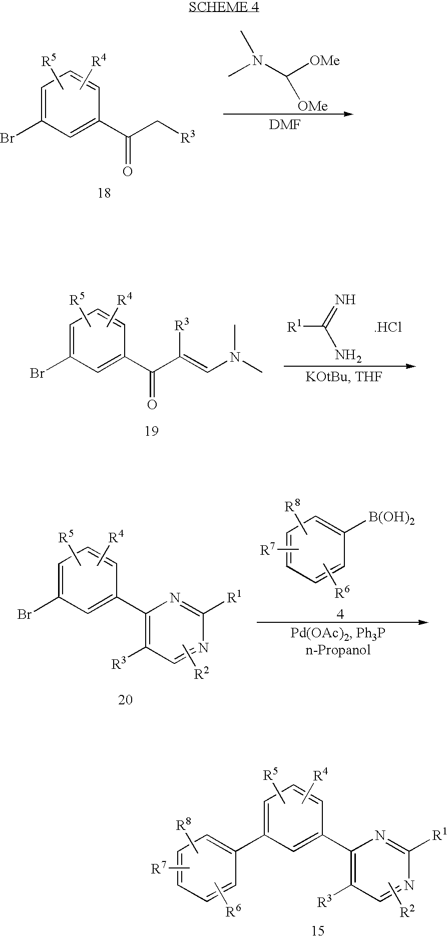

Compounds of the instant invention represented by the formula shown immediately below can be prepared from the biphenyl nitrile 22 as illustrated in

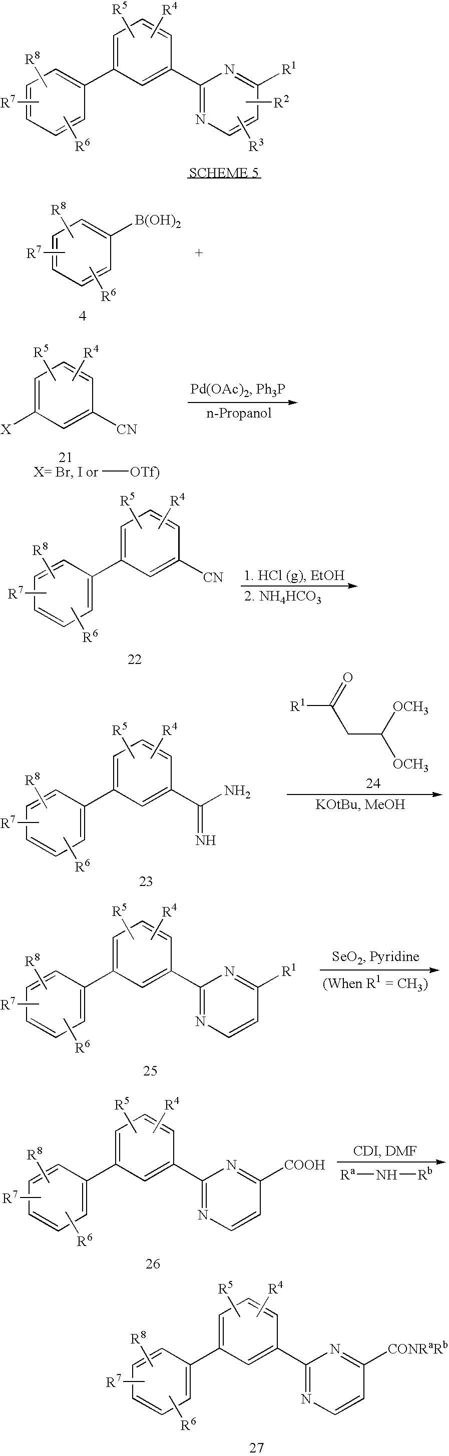

The nitrile 22 can be prepared from the Pd-catalyzed coupling of the boronic acid 4 with an appropriately substituted benzonitrile 21. The nitrile 22 can then be converted into the amidine 23 as oulined. The reaction of 23 with with an appropriate β-keto aldehyde derivative (24) can provide the desired pyrimidine 25. The R¹ substituent can be then manipulated to provide the carboxylic acid 26 and the corrsponding amides 27, as outlined.

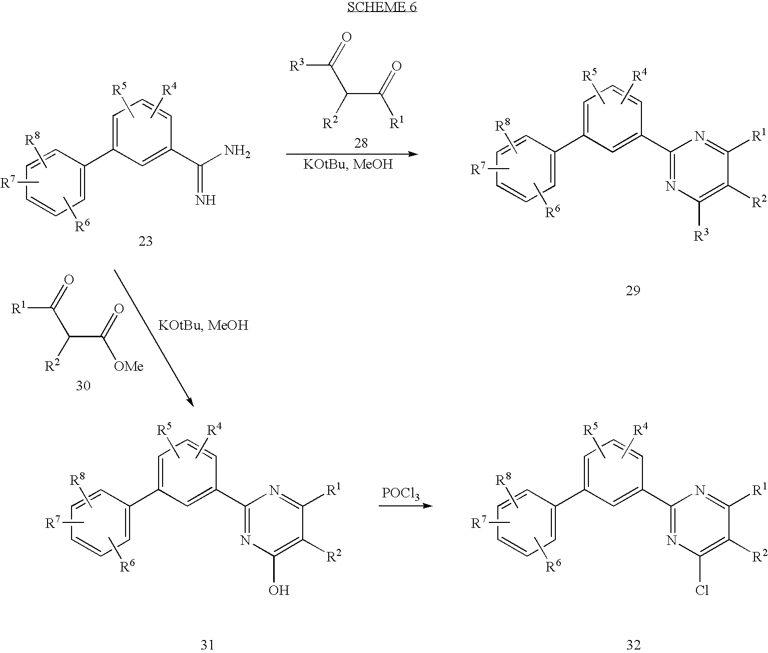

Alternatively, according to SCHEME 6, a reaction of β-diketones such as 28 with the amidine 23 may also provide a 4,6-disubstituted pyrimidine 29 (where R²═H). Similarly, the pyrimidone 31 can be synthesized by reacting an appropriate β-ketoester 30 with 23 (SCHEME 6). The pyrimidone 31 can be easily transformed into the corresponding chloro derivative 32. Replacement of the chloro group in 32 with appropriate nucleophillic reagents may provide a series analogs of 32 that can be further elaborated.

Pyrazine compounds of the present invention represented by the formula shown immediately below can be prepared as shown in SCHEME 7.

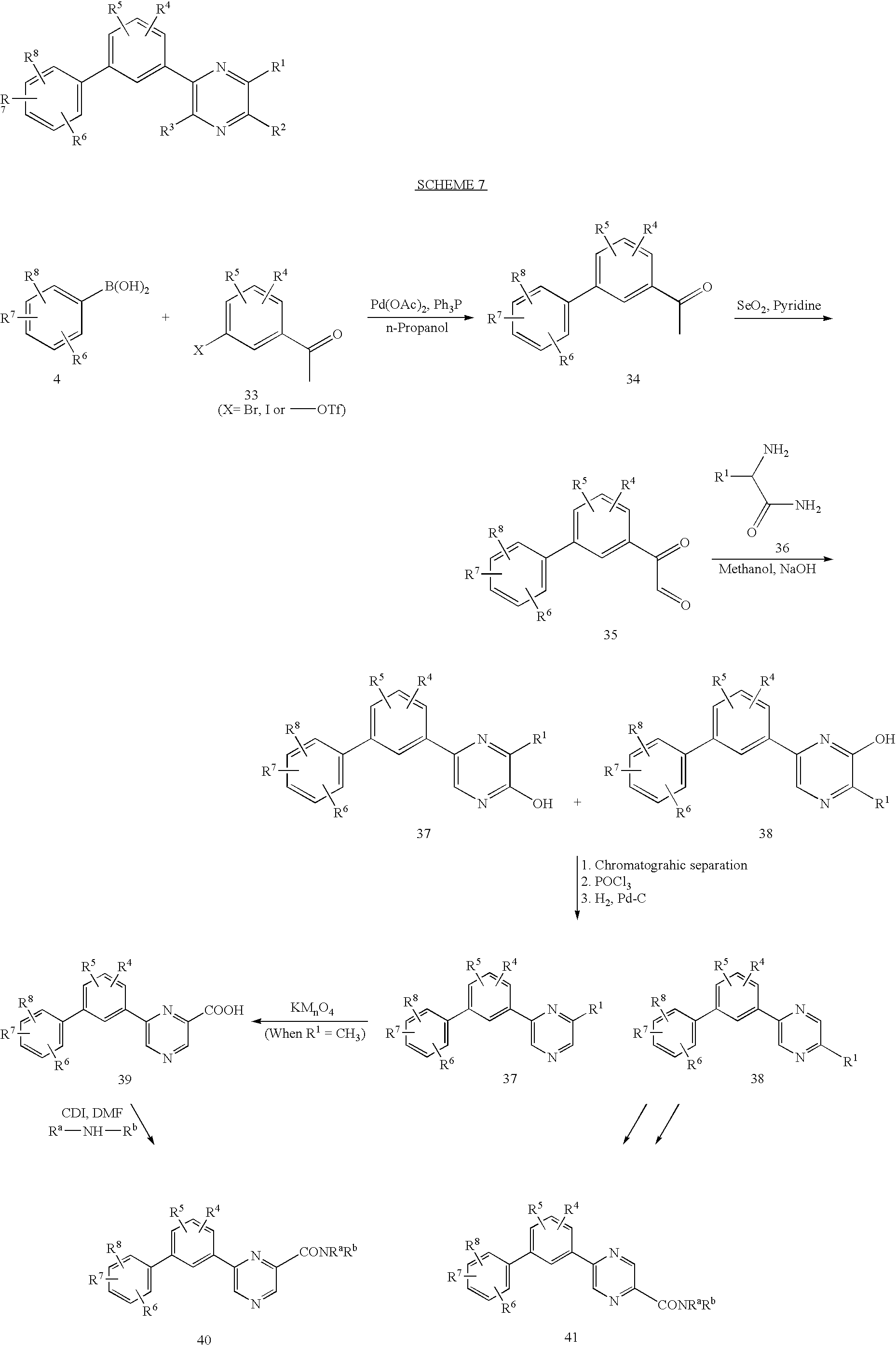

The dicarbonyl compound 35, obtained from 34, can be reacted in an appropriate solvent with an appropriate α-aminocarboxamide 36 to provide a regioisomeric mixture of pyrazinones 37 and 38, which can be separated and transformed into appropriate pyrazine derivatives such as 39, 40 and 41.

Pyrazine compounds of the instant invention represented by the formula shown immediately below can also be prepared as outlined in SCHEME 8.

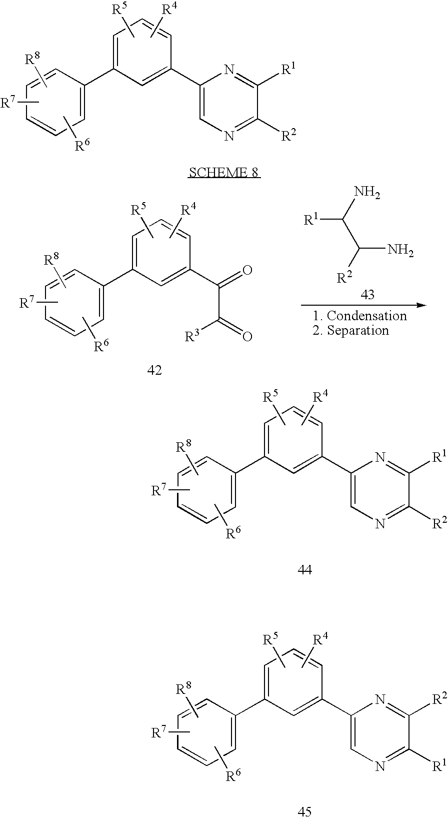

Appropriate solvents are those which will at least partially dissolve one or all of the reactants and will not adversely interact with either the reactants or the product. Suitable solvents are aromatic hydrocarbons (e.g, toluene, xylenes), halogenated solvents (e.g, methylene chloride, chloroform, carbontetrachloride, chlorobenzenes), ethers (e.g, diethyl ether, diisopropylether, tert-butyl methyl ether, diglyme, tetrahydrofuran, dioxane, anisole), nitrites (e.g, acetonitrile, propionitrile), ketones (e.g, 2-butanone, dithyl ketone, tert-butyl methyl ketone), alcohols (e.g, methanol, ethanol, n-propanol, iso-propanol, n-butanol, t-butanol), dimethyl formamide (DNM), dimethylsulfoxide (DMSO) and water. Mixtures of two or more solvents can also be used. Suitable bases are, generally, alkali metal hydroxides, alkaline earth metal hydroxides such as lithium hydroxide, sodium hydroxide, potassium hydroxide, barium hydroxide, and calcium hydroxide; alkali metal hydrides and alkaline earth metal hydrides such as lithium hydride, sodium hydride, potassium hydride and calcium hydride; alkali metal amides such as lithium amide, sodium amide and potassium amide; alkali metal carbonates and alkaline earth metal carbonates such as lithium carbonate, sodium carbonate, Cesium carbonate, sodium hydrogen carbonate, and cesium hydrogen carbonate; alkali metal alkoxides and alkaline earth metal alkoxides such as sodium methoxide, sodium ethoxide, potassium tert-butoxide and magnesium ethoxide; alkali metal alkyls such as methyllithium, n-butyllithium, sec-butyllithium, t-bultyllithium, phenyllithium, alkyl magnaesium halides, organic bases such as trimethylamine, triethylamine, triisopropylamine, N,N-diisopropylethylamine, piperidine, N-methyl piperidine, morpholine, N-methyl morpholine, pyridine, collidines, lutidines, and 4-dimethylaminopyridine; and bicyclic amines such as DBU and DABCO.

As described previously, in preparing the compositions for oral dosage form, any of the usual pharmaceutical media can be employed. For example, in the case of oral liquid preparations such as suspensions, elixirs and solutions, water, glycols, oils, alcohols, flavoring agents, preservatives, coloring agents and the like may be used; or in the case of oral solid preparations such as powders, capsules and tablets, carriers such as starches, sugars, microcrystalline cellulose, diluents, granulating agents, lubricants, binders, disintegrating agents, and the like may be included. Because of their ease of administration, tablets and capsules represent the most advantageous oral dosage unit form in which solid pharmaceutical carriers are employed. If desired, tablets may be coated by standard aqueous or nonaqueous techniques. In addition to the common dosage forms set out above, controlled release means and/or delivery devices may also be used in administering the instant compounds and compositions.

It is understood that the functional groups present in compounds described in the above schemes can be further manipulated, when appropriate, using the standard functional group transformation techniques available to those skilled in the art, to provide desired compounds described in this invention.

Other variations or modifications, which will be obvious to those skilled in the art, are within the scope and teachings of this invention. This invention is not to be limited except as set forth in the following claims.

### EXAMPLE 1

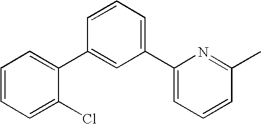 Step 1: Preparation of:

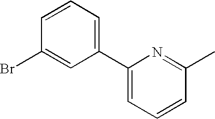

A 100-ml round-bottom flask fitted with a stirbar, condenser, and septum was flushed with N₂ and charged with 2-bromo-6-methyl pyridine (1.50 g), toluene (36 mL), deionized water (18 mL), and ethanol (18 mL). 3-bromophenylboronic acid (1.84 g) was then added to the mixture followed by sodium carbonate (1.85 g). Finally, tetrakis(triphenylphosphine) palladium (0) (0.508 g) was added to the solution quickly, and the reaction was refluxed. After two hours, the reaction was cooled to room temperature and partitioned between EtOAc and water. The aqueous layer was extracted a second time with EtOAc. The combined organic phase was dried over sodium sulfate and concentrated in vacuo. The crude material obtained was purified by column chromatography on silica gel using a gradient of 5-8% EtOAc in hexanes to yield the pure desired bromo compound.

MS: m/e 249/251 (M+1)⁺
Step 2: Preparation of

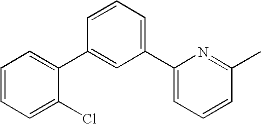

A 25-ml round-bottom flask fitted with a stirbar, condenser, and septum was flushed with N₂ and charged with the bromo compound from step 1 above (0.455 g), toluene (6 mL), deionized water (3 mL), and ethanol (3 mL). 2-chlorophenylboronic acid (572 mg) was then added followed by sodium carbonate (0.388 g). To the resulting solution, tetrakis(triphenylphosphine) palladium (0) (0.106 g) was added quickly. The reaction was refluxed for two hours and then cooled to room temperature. The mixture was partitioned between EtOAc and water. The aqueous layer was extracted a second time with EtOAc. The combined organic phase was dried over sodium sulfate and concentrated in vacuo. The crude material, thus obtained, was purified by column chromatography on silica gel using 8% EtOAc in hexanes to provide the desired biphenyl pyridine MS: m/e 280 (M+1)⁺

### EXAMPLE 2

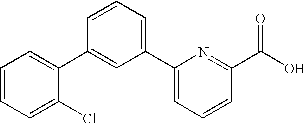

To a solution of the methyl pyridyl compound (0.475 g) from Step 2 of Example 1 and anhydrous pyridine (7 mL) was added selenium dioxide (1.30 g). The mixture was refluxed overnight (˜18 hours). An additional 8 equivalents of selenium dioxide were added and the reaction was allowed to proceed for another 30 hours. The reaction was cooled to room temperature and filtered through a pad of Celite. The filtrate was concentrated in vacuo. The crude material was purified by reverse-phase column chromatography using CH3CN-water containing 0.1% TFA to provide the desired carboxylic acid. MS: m/e 310 (M+1)⁺

### EXAMPLE 3

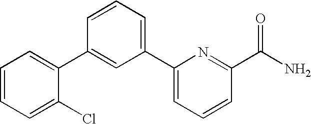

The carboxylic acid from Example 2 (0.09 g) was dissolved in anhydrous DMF (6 mL) in a 10-ml round bottom flask under N₂. Carbonyl-di-imidazole (CDI) (0.094 g) was added and the solution was stirred at room temperature for 1 hour. Solid ammonium acetate (0.089 g) was then added and stirring continued overnight at room temperature. The reaction was quenched with water (˜4mL) and extracted with 2×4 ml portions of EtOAc. The organic phase was dried over sodium sulfate and concentrated in vacuo. The crude material was then purified by column chromatography on silica gel using 50% EtOAc in hexanes to give the pure desired amide.

¹H NMR (CDCl₃): 5.89 (s, 1H), 7.36-7.42 (m, 2H), 7.47 (d, J=7.3 Hz, 1H), 7.56-7.64 (m, 3H), 7.97-8.01 (m, 2H), 8.05 (s, 1H), 8.07 (d, J=7.8 Hz, 1H), 8.15 (s, 1H), 8.23 (d, J=6.2 Hz, 1H) MS (ESI): m/e 309 (M+1)⁺

Other Examples of the instant compounds are given below in TABLE 1.

TABLE 1

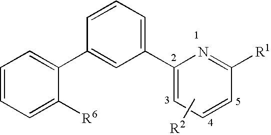

| | | | | MS |
|---|---|---|---|---|
| | | | | (m/e, |
| EXAMPLE # | R⁶ | R² | R¹ | M + 1) |
| 4 | OCF₃ | 5-CO₂CH₃ | H | 374 |
| 5 | OCF₃ | 5-CH₃ | H | 330 |
| 6 | OCF₃ | 5-COOH | H | 360 |
| 7 | OCF₃ | 4-CH₃ | H | 330 |
| 8 | OCF₃ | 4-COCH | H | 360 |
| 9 | OCF₃ | 4-CONH₂ | H | 359 |
| 10 | OCF₃ | 3-CO₂CH₃ | H | 374 |
| 11 | OCF₃ | 3-CH₃ | H | 330 |
| 12 | OCF₃ | 3-COOH | H | 360 |
| 13 | OCF₃ | 3-CONH₂ | H | 359 |
| 14 | OCF₃ | H | CH₃ | 330 |
| 15 | OCF₃ | H | COOH | 360 |
| 16 | OCF₃ | 4-CH₃ | CONH₂ | 359 |
| 17 | CF₃ | 4-COOH | H | 314 |
| 18 | CF₃ | 3-CH₃ | H | 344 |
| 19 | CF₃ | H | H | 314 |
| 20 | CF₃ | H | CH₃ | 314 |
| 21 | CF₃ | H | COOH | 344 |
| 22 | CF₃ | H | CONH₂ | 343 |
| 23 | Cl | 4-CH₃ | H | 280 |
| 24 | Cl | 4-COOH | H | 310 |
| 25 | Cl | 3-CH₃ | H | 280 |
| 26 | OCF₃ | 3-OCH₃ | H | 280 |

Further Examples of this invention are shown in TABLE 2 and TABLE 3.

TABLE 2

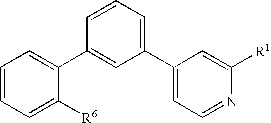

| EXAMPLE # | R⁶ | R¹ | MS (m/e, M + 1) |
|---|---|---|---|
| 27 | OCF₃ | Me | 330 |
| 28 | OCF₃ | COOH | 360 |
| 29 | OCF₃ | CONH₂ | 359 |
| 30 | CF₃ | Me | 314 |
| 31 | CF₃ | COOH | 344 |
| 32 | CF₃ | CONH₂ | 343 |

TABLE 3

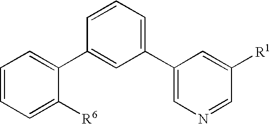

| EXAMPLE # | R⁶ | R¹ | MS (m/e, M + 1) |
|---|---|---|---|
| 33 | OCF₃ | CO₂Me | 374 |
| 34 | OCF₃ | COOH | 360 |
| 35 | OCF₃ | CONH₂ | 359 |

### EXAMPLE 36

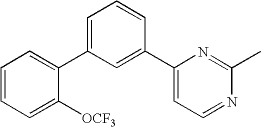 Step 1: 2-(Trifluoromethoxy)phenlboronic Acid:

n-Butyllithium (5.9 ml, 9.5 mmol) was added to a solution of 1-bromo-2-(trifluoromethoxy)benzene (2 g, 8.2 mmol) in tetrahydrofuran (28 ml) at −78° C. and stirred for 45 minutes. Triisopropyl borate (2.58 ml, 11.1 mmol) was added dropwise to the reaction mixture and the solution was slowly brought to room temperature over 16 hours. The reaction mixture was quenched with water, made basic with 2N NaOH and extracted with ethyl acetate. The aqueous solution was acidified with 2N HCl, stirred for 1 hour at room temperature and extracted into ethyl acetate. The organic layer was washed with water, brine solution and dried over sodium sulfate. It was filtered and concentrated to give the product (1.10 g, 65%) as a white solid.

¹HNMR (CDCl₃)(δ, ppm): 7.96 (dd, J=7.2, 1.6 Hz, 1 H), 7.53 (ddd, J=9.1, 7.3, 1.8 Hz, 1 H), 7.38 (td, J=7.3, 0.7 Hz, 1 H), 7.28 (d, J=8.2 Hz, 1 H), 5.25 (br s, 2H). MS (M+H): 206.9.
Step 2: Preparation of

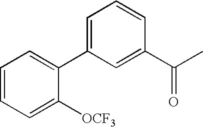

To a solution of 2-bromo(trifluoromethoxy)benzene (4.82 g, 20 mmol) (from Step 1) in n-propanol (35 mL) was added 3-acetylbenzeneboronic acid (3.61 g, 22 mmol) under N₂. After 15 min. of stirring at room temperature, Ph₃P (0.46 g, 1.7 mmol) was added followed by 2M sodium carbonate (11 mL)and water (10 mL). To the well stirred solution, palladium acetate (50 mg) was finally added quickly, and the reaction mixture was refluxed for 4 hours. The reaction was allowed to cool to room temperature and partitioned between EtOAc and water. The aqueous layer was extracted a second time with EtOAc. The combined organic phase was dried over sodium sulfate and concentrated in vacuo. The crude material, thus obtained, was purified by column chromatography on silica gel using 5% EtOAc in hexanes to yield the pure ketone as an oil. Yield: 4.45 g (79%).

NMR (CDCl₃) (δ, ppm): 8.09 (s, 1H), 8.06 (d, 1H), 7.71 (d, 2H), 7.58 (t, 1H), 7.50-7.40 (m, 4H), 2.67 (s, 3H). MS(ESI): m/e 281 (M+1)⁺
Step 3: Preparation of

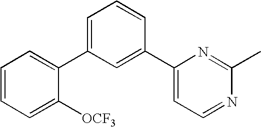

The ketone (1.12 g, 4 mmol), from Step 2 above, was dissolved in dry DMF (5 mL) and N, N-dimethyl formamide dimethyl acetal (0.59 mL, 4.2 mmol) was added. The resulting mixture was refluxed overnight. The mixture was then cooled and partitioned between EtOAc and water. The organic phase was separated, dried over sodium sulfate and concentrated in vacuo to give an orange colored solid (1.35 g, 95%). MS (ESI): ni/e 336.1 (M+1)⁺. A solution of the solid (0.335 g, 1 mmol) in anhydrous THF (2 mL) was then added to an aged acetamniidine in THF suspension (prepared by refluxing a mixture of acetamidine hydrochloride (0.177 g, 1.5 mmol) and potassium t-butoxide (0.168 g, 1.5 mmol) in THF (5 mL) for 1 hour). The orange suspension was then refluxed overnight. After cooling to room temperature, the reaction mixture was diluted in water, and extracted with EtOAc (3 times). The combined organic layer was washed with brine, and dried over anhydrous sodium sulfate. After concentration, the crude product was purified by column chromatograghy on silica gel using 33% EtOAc in hexane to afford desired product as a foam (0.28 g) in 81% yield.

¹H NMR (CDCl₃) (δ, ppm): 8.70 (d, J=5.0 Hz, 1H), 8.18 (m, 1H), 8.11 (q, J=4.5, 7.0 Hz, 1H), 7.50 (m, 3H), 7.45 (t, J=3.0 Hz, 1H), 7.34 (t, J=9.0 Hz, 1H), 7.22 (t, J=9.0 Hz, 1H), 2.82 (s, 1H). MS(ESI): m/e 331.1 (M+1)⁺

### EXAMPLE 37

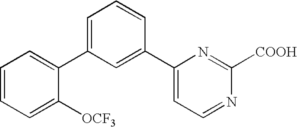

To a solution of the pyrimidine (0.27 g, 0.818 mmol), from Step 3 of Example 36, in dry pyridine (5 ml) was added SeO₂ (0.32 g, 2.8 mmol), and the mixture was refluxed overnight. The reaction was cooled to room temperature and filtered through a pad of Celite. The filtrate was concentrated in vacuo. The residue was stirred with 2N NaOH (3 mL) for 30 min and then acidified with 2N HCl. The resulting precipitate was extracted into EtOAc and the organic layer was washed with water, dried over sodium sulfate and concentrated in vacuo. The residue obtained was triturated with a 1:1 mixture of ether and hexane to give the desired carboxylic acid (0.23 g, 78%) as a cream colored solid.

¹H NMR (CDCl₃) (δ, ppm): 8.97 (d, J=5.5 Hz, 1H), 8.28 (m, 1H), 8.18 (q, J=4.5, 7.0 Hz, 1H,), 7.86 (d, J=5.5 Hz, 1H), 7.52 (m, 1H), 7.46 (t, J=7.0 Hz, 1H), 7.38 (t, J=9.0 Hz, 1H), 7.26 (t, J=9.0 Hz, 1H). MS(ESI): m/e 361.1 (M+1)⁺

### EXAMPLE 38

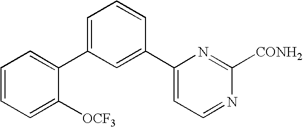

To a solution of the carboxylic acid (0.18 g, 0.5 mmol), from Example 37, in dry DMF (2 mL) was added CDI (0.1 g, 0.62 mmol), and the mixture was stirred at room temperature for 1 h. Solid dry ammonium acetate (0.5 g, 6.5 mmol) was then added and the mixture was stirred at room temperature overnight. The reaction was quenched with water (˜10 mL) and extracted with EtOAc. The organic phase was washed with water, dried over sodium sulphate and concentrated in vacuo. The crude product obtained was purified on silica-gel by radial chromatography using 75% EtOAc in hexane to yield the pure product (0.08 g, 44%) as a cream colored solid.

¹H NMR (CDCl₃) (δ, ppm): 8.89 (d, J=5.5 Hz, 1H), 8.18 (m, 1H), 8.13 (m, 1H,), 7.88 (bs, 1H), 7.79 (d, J=5.5 Hz, 1H), 7.45 (m, 1H), 7.43 (m, 1H), 7.31 (t, J=9.0 Hz, 1H), 7.18 (t, J=9.0 Hz, 1H), 6.60 (bs, 1H). MS(ESI): m/e 360.1 (M+1)⁺.

Further Examples of this invention are described in TABLE 4. These compounds were prepared employing the chemistry similar to that described in Examples 36-38.

TABLE 4

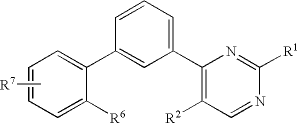

| | | | | | MS (m/e, |
|---|---|---|---|---|---|
| EXAMPLE # | R⁶ | R⁷ | R² | R¹ | M + 1) |
| 39 | OCF₃ | H | H | H | 317 |
| 40 | OCF₃ | H | H | 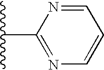 | 395 |
| 41 | OCF₃ | H | H | —SCH₃ | 363 |
| 42 | OCF₃ | H | H | —SO₂CH₃ | 395 |
| 43 | OCF₃ | H | H | —SOCH₃ | 379 |
| 44 | OCF₃ | H | H | NH₂ | 332 |
| 45 | OCF₃ | H | H | NHSO₂CH₃ | 410 |
| 46 | OCF₃ | H | H | N(SO₂CH₃)₂ | 488 |
| 47 | OCF₃ | H | H | NHCO(CH₃)₃ | 416 |
| 48 | OCF₃ | H | H | CON(CH₃)OCH₃ | 404 |
| 49 | OCF₃ | H | H | 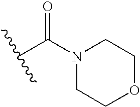 | 430 |
| 50 | OCF₃ | H | H | CH₃CO | 359 |
| 51 | OCF₃ | H | H | CONHC(CH₃)₂COOCH₃ | 460 |
| 52 | OCF₃ | H | H | CONHCH₂CH₂CN | 413 |
| 53 | OCF₃ | H | H | CONHC(CH₃)₂COOH | 446 |
| 54 | OCF₃ | H | H | CONHC(CH₃)₂CONIH₂ | 445 |
| 55 | OCF₃ | H | H | CON(CH₂CH₂)₂NH | 429 |
| 56 | OCF₃ | H | H | 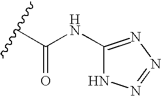 | 428 |
| 57 | OCF₃ | H | H | CONHC(CH₂)₂COOCH₃ | 458 |
| 58 | OCF₃ | H | H | CONHC(CH₂)₂COOH | 444 |
| 59 | OCF₃ | H | H | CONHC(CH₂)₂CONH₂ | 443 |
| 60 | OCF₃ | H | H | CON(CH₂)₂N(CH₃)₂ | 431 |
| 61 | OCF₃ | H | H | CONHCH₃ | 373 |
| 62 | OCF₃ | H | H | CON(CH₃)₂ | 388 |
| 63 | OCF₃ | H | H | COOCH₃ | 375 |
| 64 | OCF₃ | H | H | CONHCH(CH₃)CONH₂(S) | 431 |
| 65 | OCF₃ | H | H | 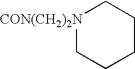 | 471 |
| 66 | OCF₃ | H | H | CONHC(CH₃)₃ | 416 |
| 67 | OCF₃ | H | H | CON(CH₃)₂CH₂OH | 431 |
| 68 | OCF₃ | H | H | CONHC(CH₃)CONH₂(R) | 431 |
| 69 | OCF₃ | H | H | CONH₂ | 457 |
| 70 | OCF₃ | H | CH₃ | CH₃ | 345 |
| 71 | OCF₃ | H | CH₃ | COOH | 375 |
| 72 | OCF₃ | H | CH₃ | CONH₂ | 374 |
| 73 | OCF₃ | H | H | CONHCH₂CONH₂ | 417 |
| 74 | OCF₃ | H | Cl | CH₃ | 365 & 367 |
| 75 | OCF₃ | H | Cl | CONH₂ | 394 & 396 |
| 76 | OCF₃ | H | H | NHCONH₂ | 409 |
| 77 | CF₃ | H | H | CH₃ | 315 |
| 78 | CF₃ | H | H | H | 301 |
| 79 | CF₃ | H | H | COOH | 345 |
| 80 | CF₃ | H | H | CONH₂ | 344 |
| 81 | CF₃ | H | H | 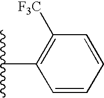 | 445 |
| 82 | CF₃ | H | H | SH | 333 |
| 83 | CF₃ | H | H | S—COCH₃ | 375 |
| 84 | CF₃ | H | H | Cl | 335 & 337 |
| 85 | CF₃ | H | H | CN | 326 |
| 86 | CF₃ | H | H | 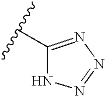 | 369 |
| 87 | CF₃ | 5-F | H | CH₃ | 333 |
| 88 | CF₃ | 5-F | H | COOH | 363 |
| 89 | CF₃ | 5-F | H | CONH₂ | 362 |
| 90 | CF₃ | 4-CF₃ | H | CH₃ | 383 |
| 91 | CF₃ | 4-CF₃ | H | COOH | 413 |
| 92 | CF₃ | 4-CF₃ | H | CONH₂ | 412 |
| 93 | CF₃ | 4-CF₃ | H | 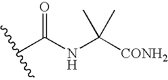 | 497 |
| 94 | O-Ph | H | H | CH₃ | 339 |
| 95 | O-Ph | H | H | COOH | 369 |
| 96 | O-Ph | H | H | CONH₂ | 368 |
| 97 | H | O-Ph | H | CONH₂ | 368 |
| 98 | Cl | H | H | CH₃ | 281 |
| 99 | H | 3-Cl | H | CH₃ | 281 |
| 100 | —SO₂NH— | H | H | CH₃ | 382 |
| | tBu | | | | |
| 101 | —SO₂NH₂ | H | H | CH₃ | 326 |
| 102 | —CONH— | H | H | CH₃ | 346 |
| | tBu | | | | |
| 103 | —CONH₂ | H | H | CH₃ | 290 |
| 104 | —CONH— | H | H | COOH | 376 |
| | tBu | | | | |
| 105 | —CONH— | H | H | CONH₂ | 375 |
| | tBu | | | | |
| 106 | Cl | 3-Cl | H | COOH | 344 |
| 107 | Cl | 3-Cl | H | CONH₂ | 343 |
| 108 | Cl | 3-Cl | H | COOCH₃ | 359 |
| 109 | —SO₂NH— | H | H | COOH | 412 |
| | tBu | | | | |
| 110 | —SO₂NH₂ | H | H | COOH | 356 |
| 111 | —SO₂NH— | H | H | CONH₂ | 411 |
| | tBu | | | | |
| 112 | —SO₂NH₂ | H | H | CONH₂ | 355 |
| 113 | OtBu | H | H | CH₃ | 319 |
| 114 | OtBu | H | H | COOH | 349 |
| 115 | OtBu | H | H | CONH₂ | 348 |
| 116 | 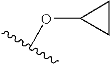 | H | H | CH₃ | 303 |
| 117 | 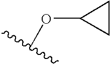 | H | H | COOH | 333 |
| 118 | 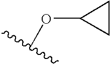 | H | H | CONH₂ | 332 |
| 119 | OCH₂CF₃ | H | H | CH₃ | 345 |
| 120 | OCH₂CF₃ | H | H | COOH | 375 |
| 121 | OCH₂CF₃ | H | H | CONH₂ | 374 |
| 122 | CHO | H | H | CONH₂ | 304 |
| 123 | H | 3-CF₃ | H | CONH₂ | 344 |
| 124 | H | 4-CF₃ | H | CONH₂ | 344 |
| 125 | H | 3-F | H | CONH₂ | 294 |
| 126 | H | 4-Cl | H | CONH₂ | 310 |
| 127 | H | 4-F | H | CONH₂ | 294 |
| 128 | 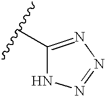 | H | H | CONH₂ | 344 |
| 129 | OCH₃ | 3-OCH₃ | H | CONH₂ | 336 |
| 130 | OCH₃ | 5-Cl | H | CONH₂ | 340 |
| 131 | CH₃ | H | H | CONH₂ | 290 |
| 132 | CH₃ | 3-F | H | CONH₂ | 308 |
| 133 | 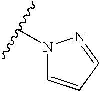 | H | H | CONH₂ | 342 |
| 134 | H | 4-(CH₂OH) | H | CONH₂ | 306 |
| 135 | H | 3-Cl | H | CONH₂ | 310 |
| 136 | H | 3-OHt | H | CONH₂ | 320 |
| 137 | H | 4-OHt | H | CONH₂ | 320 |
| 138 | F | H | H | CONH₂ | 294 |
| 139 | CH₃ | 6-CH₃ | H | CONH₂ | 304 |
| 140 | H | 4-tBu | H | CONH₂ | 332 |
| 141 | H | 4-OCF₃ | H | CONH₂ | 360 |
| 142 | H | 4-COCH₃ | H | CONH₂ | 318 |
| 143 | H | 3-COCH₃ | H | CONH₂ | 318 |
| 144 | H | 3-(CH₂OH) | H | CONH₂ | 306 |
| 145 | H | 4-CN | H | CONH₂ | 301 |
| 146 | H | 3-OCF₃ | H | CONH₂ | 360 |
| 147 | F | 4-F | H | CONH₂ | 312 |
| 148 | H | H | H | CONH₂ | 276 |
| 149 | OCF₃ | 4- | H | CH₃ | 438 |
| | | N(Me)SO₂Me | | | |
| 150 | OCF₃ | 4- | H | CONH₂ | 467 |
| | | N(Me)SO₂Me | | | |
| 151 | OCF₃ | 4-NHCO-tBu | H | CH₃ | 430 |
| 152 | OCF₃ | 4-NHCO-tBu | H | COOH | 460 |
| 153 | OCF₃ | 4-NHCO-tBu | H | CONH₂ | 459 |
| 154 | OCF₃ | H | H | 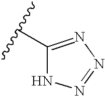 | 385 |
| 155 | OCF₃ | H | H | 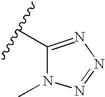 | 399 |
| 156 | OCF₃ | H | H | 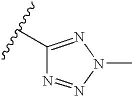 | 399 |
| 157 | OCF₃ | H | H | 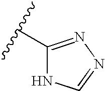 | 384 |
| 158 | OCF₃ | H | H | —CH₂CONH₂ | 374 |
| 159 | OCF₃ | H | H | —CH₂CN | 356 |
| 160 | OCF₃ | H | H | —SO₂NHtBu | 452 |
| 161 | OCF₃ | H | H | —SO₂NH₂ | 396 |
| 162 | OCF₃ | H | H | —SO₂NHMe | 410 |
| 163 | OCF₃ | H | H | —CH₂OH | 347 |
| 164 | OCF₃ | H | H | —CH(Me)OH | 361 |
| 165 | OCF₃ | H | H | —CH₂NHCOCH₃ | 388 |
| 166 | OCF₃ | H | H | —CH₂OSO₂NH₂ | 426 |
| 167 | OCF₃ | H | H | —NHCH₃ | 346 |
| 168 | OCF₃ | H | H | —NH—CH(CH₃)₂ | 374 |
| 169 | OCF₃ | H | H | 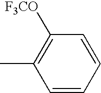 | 477 |

Further Examples of this invention are described in TABLE 5.

TABLE 5

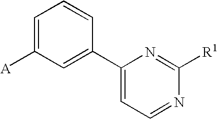

| | | | MS (m/e, |
|---|---|---|---|
| EXAMPLE # | A | R¹ | M + 1) |
| 170 | 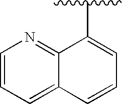 | CONH₂ | 328 |
| 171 | 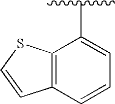 | CONH₂ | 332 |
| 172 | 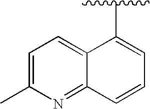 | CONH₂ | 343 |
| 173 | 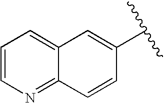 | CONH₂ | 328 |
| 174 | 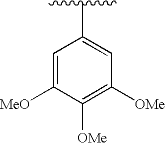 | CONH₂ | 366 |
| 175 | 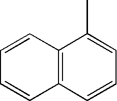 | CONH₂ | 328 |
| 176 | 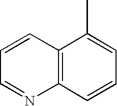 | CONH₂ | 329 |
| 177 | 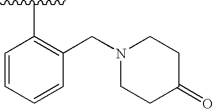 | CONH₂ | 387 |
| 178 | 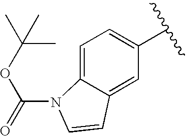 | CONH₂ | 415 |

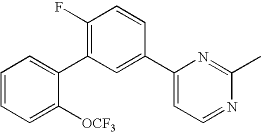 Step A: Preparation of 2-methyl-4-(3-bromo-4-fluoro phenyl)-pyrimidine

To the solution of 3-bromo-4-fluoroacetophenone (434 mg, 2 mmol) in DMF (5 mL) was added N, N-dimethyl formamide dimethyl acetal (0.41 mL, 3 mmol). The resulting solution was stirred at room temperature overnight. After removal of the solvent and excess reagent, the residue was dissolved in anhydrous THF, and teated with aged acetamidine in THF suspension (a mixture of acetamidine hydrochloride (283 mg, 3 mmol) and potassium t-butoxide (336 mg, 3 mmol) in THF (10 mL), reflux 1 hour). The orange suspension was then refluxed overnight. After cooling to room temperature, the reaction mixture was diluted in water, and extracted with EtOAc (3 times). The combined organic layer was washed with brine, and dried over anhydrous sodium sulfate. After concentration, the crude product was applied to column chromatographyon silica gel to afford the final product as a yellow solid, 400 mg, 75% yield. The above product was used for the Suzuki coupling in the next step,

Step B: Coupling of 2-methyl-4-(3-bromo-4-fluorophenyl)-pyrimidine with 2-trifluoromethoxyphenyl Boronic Acid

To the solution of 2-trifluoromethoxyphenyl boronic acid (216 mg, 1.05 mmol) and the bromophenyl compound (200 mg, 11.6 mmol) in n-propanol (5 mL) was added palladium acetate (35 mg, 0.15 mmol), triphenyl phosphine (118 mg, 0.45 mmol), and aqueous sodium carbonate (2.0M, 0.45 mL, 0.9 mmol). The reaction mixture was stirred at 90° C. for 16 hours. After cooling to room temperature, the mixture was filtered through a Celite pad, and washed with ethyl acetate (3 times). The filtrate was concentrated. The resulting residue was dissolved in ethyl acetate and washed with saturated sodium carbonate aqueous solution and brine, the organic layer was dried over anhydrous sodium sulfate. After concentration, the crude product was applied to column chromatographyon silica gel to afford the final the titled compound, as a white solid. ¹H NMR (CDCl₃) (δ, ppm): 8.70 (d, J=5.0 Hz, 1H), 8.18 (m, 1H), 8.11 (q, J=4.5, 7.0 Hz, 1H), 7.50 (m, 3H), 7.45 (t, J=3.0 Hz, 1H), 7.34 (t, J=9.0 Hz, 1H), 7.22 (t, J=9.0 Hz, 1H), 2.82 (s, 1H). MS (ESI): m/e 349 (M+1)⁺

### EXAMPLE 180

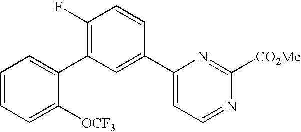

To the solution of 2-methylpyrimidine(from Example 179) (70 mg, 0.21 mmol) in pyridine (3 ml) was added selenium dioxide (117 mg, 1.1 mmol). The resulting yellow solution was refluxed for 20 hours. After cooling to room temperature, the solvent was removed under reduced pressure. The residue was partitioned between ethyl acetate and 2N HCl. The aqueous layer was extracted with ethyl acetate. The combined organic layer was washed with brine and dried over anhydrous sodium sulfate. The crude acid was dissolved in methanol, and treated with excess 2.0M trimethylsilyldiazomethane in methanol solution at room temperature for 10 minutes. After concentration, the titled compound was isolated via column chromatography on silica gel, as a yellow solid.

¹H NMR (CDCl₃) (δ, ppm): 8.97 (d, J=5.5 Hz, 1H), 8.28 (m, III), 8.18 (q, J=4.5, 7.0 Hz, 1H,), 7.86 (d, J=5.5 Hz, 1H), 7.52 (m, 1H), 7.46 (t, J=7.0 Hz, 1H), 7.38 (t, J=9.0 Hz, 1H), 7.26 (t, J=9.0 Hz, 1H), 4.12 (s, 1H). MS (ESI): m/e 393 (M+1)

### EXAMPLE 181

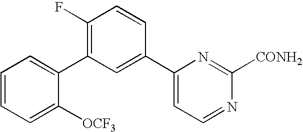

The pyrimidine methyl ester (from Example 180) (120 mg, 0.31 mmol) in ammonium-methanol (2.0M, 3 mL), was stirred at 70° C. in a sealed tube. The reaction was stirred at that temperature for overnight. After cooling down, the reaction mixture was concentrated to give the titled compound as yellow foam.

¹HNMR (CDCl₃) (δ, ppm): 8.89 (d, J=5.5 Hz, 1H), 8.18 (m, 1H), 8.13 (m, 1H,), 7.88 (bs, 1H), 7.79 (d, J=5.5 Hz, 1H), 7.45 (m, 1H), 7.43 (m, 1H), 7.31 (t, J=9.0 Hz, 1H), 7.18 (t, J=9.0 Hz, 1H), 6.60 (bs, 1H). MS (ESI): m/e 378 (M+1)⁺

Further Examples of this invention are shown below in TABLE 6.

TABLE 6

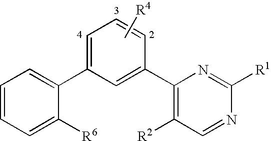

| | | | | | MS (m/e, |
|---|---|---|---|---|---|
| EXAMPLE # | R⁶ | R⁴ | R² | R¹ | M + 1) |
| 182 | OCF₃ | 4-F | H | CH₃ | 349 |
| 183 | OCF₃ | 4-F | H | COOH | 379 |
| 184 | OCF₃ | 4-F | H | COOCH₃ | 393 |
| 185 | OCF₃ | 4-F | H | CONH₂ | 378 |
| 186 | CF₃ | 4-F | H | COOCH₃ | 377 |
| 187 | CF₃ | 4-F | H | CONH₂ | 362 |
| 188 | CF₃ | 4-F | H | CH₃ | 351 |
| 189 | OCF₃ | 2-OCH₂Ph | H | CH₃ | 437 |
| 190 | OCF₃ | 2-OH | H | CH₃ | 347 |
| 191 | OCF₃ | 4-NHAc | H | CH₃ | 386 |
| 192 | OCF₃ | 4-NHAc | H | COOCH₃ | 432 |
| 193 | OCF₃ | 4-NHAc | H | CONH₂ | 417 |
| 194 | OCF₃ | 2-F | H | CH₃ | 349 |
| 195 | OCF₃ | 2-F | H | COOCH₃ | 393 |
| 196 | OCF₃ | 2-F | H | CONH₂ | 378 |
| 197 | OCF₃ | 4-Br | H | CH₃ | 410 |
| 198 | OCF₃ | 4-Br | H | COOCH₃ | 454 |
| 199 | OCF₃ | 4-Br | H | CONH₂ | 439 |
| 200 | OCF₃ | 4-Br | H | COOH | 440 |
| 201 | OCF₃ | 4-Ph | H | CH₃ | 407 |
| 202 | OCF₃ | 4-Ph | H | COOCH₃ | 451 |
| 203 | OCF₃ | 4-Ph | H | CONH₂ | 436 |
| 204 | OCF₃ | 4-Cl | H | CH₃ | 365 |
| 205 | OCF₃ | 4-Cl | H | COOCH₃ | 409 |
| 206 | OCF₃ | 4-Cl | H | COOH | 395 |
| 207 | OCF₃ | 4-Cl | H | CONH₂ | 394 |
| 208 | OCF₃ | 2-Cl | H | CH₃ | 365 |
| 209 | OCF₃ | 2-Cl | H | COOCH₃ | 409 |
| 210 | OCF₃ | 2-Cl | H | CONH₂ | 394 |
| 211 | OCH₂CF₃ | 4-F | H | CH₃ | 363 |
| 212 | OCH₂CF₃ | 4-F | H | COOCH₃ | 407 |
| 213 | OCH₂CF₃ | 4-F | H | COOH | 393 |
| 214 | OCH₂CF₃ | 4-F | H | CONH₂ | 392 |
| 215 | H | 4- | H | CONH₂ | 373 |
| | | OCH₂CF₃ | | | |
| 216 | F | 4- | H | CONH₂ | 392 |
| | | OCH₂CF₃ | | | |

### EXAMPLE 217

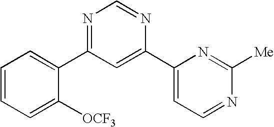 Step 1A: Preparation of 4-chloro-6-methoxypyrimidine

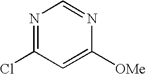

To the solution of 4,6-dichloropyrimidine (2 g, 13.4 mmol) in methanol (20 mL), was added sodium methoxide (25% w/w, 3.1 mL, 13.4 mmol). The white precipitate was formed immediately. 30 minutes later the reaction mixture was filtrated through a Celite pad, the filter cake was washed with ethyl acetate. The filtrate was then concentrated, and applied to column chromatoghraphy on silica gel to afford the titled compound as a white crystalline solid.

Step 1B: Coupling of 4-chloro-6-methoxypyrimidine with 2-trifluoromethoxyphenylboronic Acid

To the solution of 2-trifluoromethylphenyl boronic acid (1.74 g, 9.1 mmol) and the 4-chloro-6-methoxypyrimidine (940 mg, 6.5 mmol) in n-propanol (15 mL) was added palladium acetate (292 mg, 1.3 mmol), triphenyl phosphine (1 g, 4 mmol), and aqueous sodium carbonate (2.0M, 4 mL, 7.8 mmol). The reaction mixture was stirred at 90° C. for 16 hours. After cooling to room temperature, the mixture was filtered through a Celite pad, and washed with ethyl acetate (3 times). The filtrate was concentrated. The resulting residue was dissolved in ethyl acetate, and washed with saturated sodium carbonate aqueous solution and brine. The organic layer was dried over anhydrous sodium sulfate. After concentration, the crude product was applied to column chromatographyon silica gel to afford the titled compound as yellow oil.

¹H NMR (CDCl₃) (δ, ppm): 8.83 (s, 1H), 7.75 (d, J=8.0 Hz, 1H,), 7.61 (t, J=8.0 Hz, 1H), 7.54 (t, J=7.5 Hz, 1H), 7.45 (t, J=7.5 Hz, 1H), 6.83 (s, 1H), 4.02 (s, 1H). MS (ESI): m/e 255 (M+1)⁺
Step 2: Preparation of

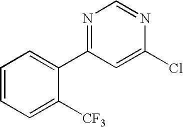

To the solution of the 4-(2-trifluoromethylbenzene)-6-methoxypyrimidine (from Step B of Step 1) (45 mg, 0.18 mmol) in acetic acid (1.5 mL) was added HBr (0.5 mL). The resulting colorless solution was stirred at 80° C. for 1 hour. After cooling to room temperature, the solvent was removed under reduced pressure, the residue was partitioned between ethyl acetate and saturated sodium bicarbonate aqueous solution. The aqueous layer was extracted with ethyl acetate. The combined organic layer was washed with brine, and dried over anhydrous sodium sulfate. The crude product was used immediately for the next step. The above pyrimidone was dissolved in POCl₃ (5 mL). The reaction mixture was refluxed for 30 minutes. After removing the solvent, the residue was partitioned between ethyl acetate and saturated sodium bicarbonate aqueous solution. The combined organic layer was washed with brine, and dried over anhydrous sodium sulfate. The titled compound was isolated via column chromatography on silica gel, as a yellow solid.

¹HNMR (CDCl₃) (δ, ppm): 9.06 (s, 1H), 7.80(d, J=4.0 Hz, 1H), 7.75 (t, J=8.0 Hz, 1H), 7.61 (t, J=7.5 Hz, 1H), 7.45 (t, J=7.0 Hz, 1H), 7.24 (s, 1H). MS (ESI): m/e 259 (M+1)⁺
Step 3: Preparation of

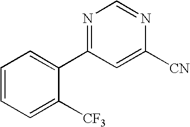

To the solution of the chloropyrimidine (from Step 2) (300mg, 1.2 mmol) in DMF (5 mL), was added potassium cyanide (117 mg, 1.7 mmol) and p-tosylate sodium salt (83 mg, 0.46 mmol). The resulting mixture was stirred at 80° C. for 2 hours. After cooling to room temperature, and removing the solvent under reduced pressure, the residue was partitioned between ethyl acetate and water. The aqueous was extracted with ethyl acetate, the organic layer was washed with brine, and dried over anhydrous sodium sulfate. After concentration, the titled compound was collected as a yellow solid.

¹H NMR (CDCl₃) (δ, ppm): 9.41 (s, 1H), 7.83 (d, J=7.5 Hz, 1H), 7.78 (s, 1H), 7.70-7.64 (m, 2H), 7.50 (d, J=7.5 Hz, 1H). MS (ESI): m/e 250 (M+1)⁺
Step 4: Preparation of

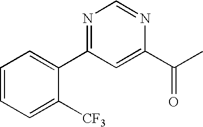

To the solution of the cyano compound (from Step 3) (160 mg, 0.64 mmol) in dry ether (5 mL) was added dropwise, at −78° C., the methyl magnesium bromide in ether solution (3.0 m, 0.64 mL, 1.9 mmol). The reaction mixture was stirred at −78° C. for 1 hour, and at room temperature for another 1 hour. The reaction mixture was partitioned between ether and water. The aqueous layer was extracted with ethyl acetate. The combined organic layer was washed with brine, and dried over anhydrous sodium sulfate. After concentration, the titled compound was collected as a yellow solid.

¹HNMR (CDCL₃) (δ, ppm): 9.41 (s, 1H), 8.02 (s, 1H), 7.81 (d, J=7.0 Hz, 1H), 7.65 (d, J=7.0 Hz, 1H), 7.61 (d, J=7.0 Hz, 1H), 7.48 (d, J=7.0 Hz, 1H), 2.76 (s, 1H). MS (ESI): M/E 267 (M+1)⁺
Step 5: Preparation of

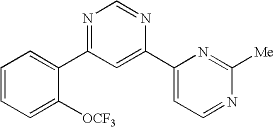

To the solution of methylketone (from Step 4) (50 mg, 0.19 mmol) in DMF (2 mL) was added N, N-dimethyl formamide dimethyl acetal (0.034 mL, 0.28 mmol). The resulting solution was stirred at room temperature for overnight. After removal of the solvent and excess reagent, the residue was dissolved in anhydrous TBF, and teated with aged acetamidine in THF suspension (a mixture of acetamidine hydrochloride (26 mg, 0.28 mmol) and potassium t-butoxide (32 mg, 0.28 mmol) in TBF (5 mL), reflux 1 hour). The orange suspension was then refluxed for overnight. After cooling to room temperature, the reaction mixture was diluted in water, and extracted with EtOAc (3 times). The combined organic layer was washed with brine, and dried over anhydrous sodium sulfate. After concentration, the crude product was applied to column chromatography on silica gel to afford the titled compound as a yellowish solid.

¹HNMR (CDCL₃) (δ, ppm): 9.38 (s, 1H), 8.86 (d, J=5.5 Hz, 1H), 8.58 (s, 1H), 8.25 (d, J=5.5 Hz, 1H), 7.82 (d, J=8.0 Hz, 1H), 7.68 (t, J=7.5 Hz, 1H), 7.59 (t, J=7.5 Hz, 1H), 7.55 (d, J=5.5 Hz, 1H), 2.80 (s, 1H). MS (ESI): M/E 317 (M+1)⁺

### EXAMPLE 218

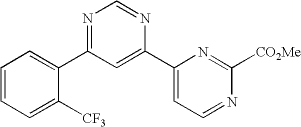

To the solution of methylpyrimidine (form Example 217, Step 5) (50 mg, 0.15 mmol) in pyridine (2 mL), was added selenium dioxide (166 mg, 1.5 mmol). The resulting yellow solution was refluxed for 20 hours. After cooling to room temperature, the solvent was removed under reduced pressure. The residue was partitioned between ethyl acetate and 2N HCl. The aqueous layer was extracted with ethyl acetate. The combined organic layer was washed with brine, and dried over anhydrous sodium sulfate. The crude acid was dissolved in MeOH, and treated with excess 2.0M trimethylsilyldiazomethane in methanol solution at room temperature for 10 minutes. After concentration, the titled compound was isolated via column chromatography on silica gel, as a yellow solid.

¹HNMR (CDCL₃) (δ, ppm): 9.45 (s, 1H), 9.18 (d, J=5.0 Hz, 1H), 8.68 (m, 2H), 7.83 (d, J=8.0 Hz, 1H), 7.68 (t, J=7.5 Hz, 1H), 7.55 (t, J=7.5 Hz, 1H), 7.54 (d, J=5.5 Hz, 1H), 4.06 (s, 1H). MS (ESI): m/e 361 (M+1)⁺

### EXAMPLE 219

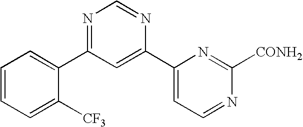

The pyrimidine methyl ester (from Example 218) (14 mg, 0.04 mmol) in ammonium-methanol (2.0M, 2 mL), was stirred at 70° C. in a sealed tube. The reaction was stirred at that temperature for overnight. After cooling down, the reaction mixture was concentrated to give the titled compound as yellow foam.

1HNMR (CDCL₃) (δ, ppm): 9.39 (s, 1H), 9.10 (d, J=5.0 Hz, 1H), 8.60 (s, 1H), 8.57 (d, J=5.0 Hz, 1H), 7.86 (bs, 1H), 7.77 (d, J=8.0 Hz, 1H), 7.64 (t, J=7.5 Hz, 1H), 7.58 (t, J=7.5 Hz, 1H), 7.52 (d, J=5.5 Hz, 1H), 6.94 (bs, 1H). MS (ESI): M/E 346 (M+1)⁺.

Further Examples of this invention were synthesized using the same procedures described in Examples 217-219 and are summarized in TABLE 7.

TABLE 7

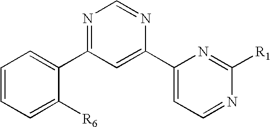

| EXAMPLE # | R₆ | R₁ | MS (m/e, M + 1) |
|---|---|---|---|
| 220 | OCF₃ | CH₃ | 333 |
| 221 | OCF₃ | COOH | 363 |
| 222 | OCF₃ | CONH₂ | 362 |

### EXAMPLE 223

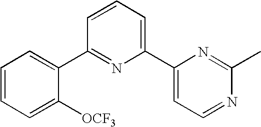 Step 1: Preparation of

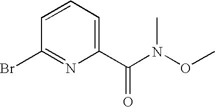

To a solution of 6-bromopicolinic acid (2.0 g) in anhydrous DMF (10 mL) was added carbonyl diimidazole (2.4 g), and the solution was stirred at room temperature for 1 hour. N,O-dimethylhydroxyl-amine hydrochloride (1.5 g) was then added and the reaction was stirred overnight at room temperature. The reaction, after quenching with water (30 mL), was extracted with 2×20 ml portions of EtOAc. The organic phase was dried over sodium sulfate and concentrated in vacuo. The crude material was purified by column chromatography on silica gel using 50% EtOAc in hexanes to give the pure amide.

¹HNMR (CDCL₃) (6, ppm): 7.70-7.61 (m, 2H), 7.59 (t, J=7.5 Hz, 1H), 3.85 (s, 3H), 3.4 (s, 3H). MS: m/e 245/247 (M+1)⁺
Step 2: Preparation of

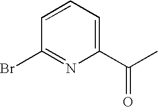

A solution of the amide (from Step 1) (2.3 g) in anhydrous THF (˜3 ml) was cooled to 0° C., and methylmagnesiumchloride (9.4 ml) was added. After stirring for 1 h at 0° C., the reaction was poured into 5% HCl in ethanol, and the mixture was partitioned between brine and a 1:1 ether and methylene chloride. The organic phase was separated and dried over sodium sulfate and concentrated in vacuo. The material was used in the next step without any purification.

¹HNMR (CDCL₃) (δ, ppm): 8.03 (dd, J₁=1.2 Hz and J₂=7.0 Hz, 1H), 7.72 (m, 2H), 2.74 (s, 3H). MS: m/e 200/2 (M+1)⁺
Step 3: Preparation of

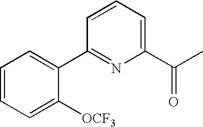

To a solution of the ketone (from step 2) (0.8 g) in a mixture of toluene (15 mL), 8 ml of ethanol (8 mL), and deionized water (8 mL) was added 2-trifluoromethoxyphenylboronic acid (0.824 g) under N₂. Sodium carbonate (0.848 g) was added to the solution followed by tetrakistriphenylphosphine palladium (0.231 g). The reaction was refluxed for 2 h, cooled to room temperature and partitioned between EtOAc and water. The aqueous layer was extracted a second time with EtOAc. The combined organic phase was dried over sodium sulfate and concentrated in vacuo. The crude material obtained was purified by column chromatography on silica gel using 15% EtOAc in hexanes to yield the pure ketone.

¹HNMR (CDCL₃) (δ, ppm): 8.03 (dd, 1H), 7.93 (dd, 1H), 7.88 (d, 1H), 7.87 (s, 1H, 7.45 (m, 2H), 7.39 (m, 1H), 2.78 (s, 3H). MS: mn/e 282 (M+1)⁺
Step 4: Preparation of

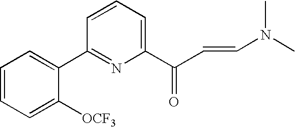

To a solution of the ketone from Step 3 (0.96 g) in DM (3.5 mL) was added N,N-dimethylformamide dimethyl acetal (0.44 g), and the mixture was stirred at 150° C. for 18 h. The reaction was then cooled to room temperature and partitioned between EtOAc and water. The aqueous layer was extracted a second time with EtOAc. The combined organic phase was dried over sodium sulfate and concentrated in vacuo. The crude material obtained was used in the next step without purification.

MS: m/e 337 (M+1)⁺
Step 5: Preparation of

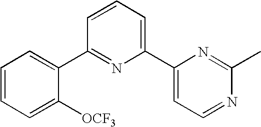

Acetamidine hydrochloride (0.51 g), anhydrous DMF (2 ml) and potassium t-butoxide (0.605 g) were placed in a 5 ml-microwave reaction tube fitted with a stirbar. A solution of the product from step 4 (1.2 g) in anhydrous DUT (2 mL) was added to the content in tube. The reaction vessel was sealed and heated 140° C. for 20 min. The microwave tube was cooled, and the reaction was partitioned between EtOAc and water. The organic phase was washed with water, dried over sodium sulfate and concentrated in vacuo. The crude material was purified by column chromatography on silica gel using 25% EtOAc in hexanes.

¹HNMR (CDCL₃) (δ, ppm): 8.78 (d, J=5.3 Hz, 1H), 8.52 (dd, J=0.9 Hz and 7.8 Hz. 1H), 8.28 (d, J=5.0 Hz, 1H), 7.92-7.98 (m, 2H), 7.80 (dd, J=0.9 Hz and 7.8 Hz. 1H), 7.42-7.5 (m, 2H), 7.38-7.43 ( m, 1H), 2.85 (s, 3H). MS: m/e 332 (M+1)⁺

### EXAMPLE 224

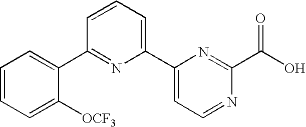

A mixture of the methyl pyrimidine, from Example 223, (0.4 g), SeO2 (2.0 g) and anhydrous pyridine (16 mL) was refluxed overnight. The reaction was filtered through Celite and the filtrate was concentrated in vacuo. The residue obtained was dissolved in EtOAc and washed with 1 N HCl. The organic phase, after drying over sodium sulfate, was concentrated in vacuo. The crude product was purified by reverse-phase column chromatography using CH3CN-water containing 0.1% TFA to give the desired product.

NMR (CDCl3): MS: m/e 362 (M+1)⁺

### EXAMPLE 225

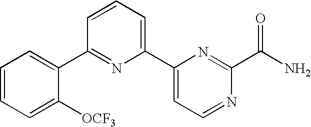

To a solution of the acid (from Example 215) (0.2 g) in anhydrous DMF (1 mL) was added carbonyldiimidazole (0.178 g), and the solution was stirred at room temperature for 1 hour. Anhydrous ammonium acetate (0.17 g) was then added and the reaction was stirred overnight. The reaction was poured into water (10 mL) and extracted with EtOAc. The organic phase was dried over sodium sulfate and concentrated in vacuo. The crude product obtained was purified by column chromatography on silica gel using 100% EtOAc in hexanes to give the pure amide.

NMR(CDCl3): MS: m/e 361 (M+1)⁺

### EXAMPLE 226

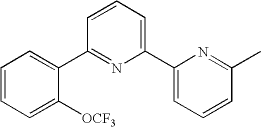 Step 1: Preparation of

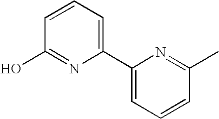

To a solution of 6-methyl-2,2′-dipyridyl (1.0 g) in CH₃CN (12 mL) was added iodomethane (5.0 g) and the reaction refluxed for two days. The reaction was cooled to room temperature and filtered. The filtrate was diluted with ether, and the precipitate formed (mono-methylated desired product) was filtered, washed with ether and dried in vacuo.

To a cold solution of potassium ferricyanide (III) (4.4 g) in water (22 ml) were added cold solutions of sodium hydroxide (4.5 g) (in water (17.5 ml)) and the above solid (1.04 g) (in water (17.5 ml)). The reaction was kept at 5° C. for 4 hours and then extracted with dichloromethane. The product was purified by column chromatography on silica gel using 20% methanol in EtOAc. MS: m/e 201 (M+1)⁺
Step 2: Preparation of

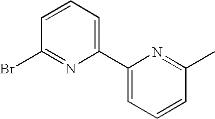

To a mixture of triphenylphosphine (0.682 g) and dry acetonitrile (7 ml) was added Br₂ (0.384 g) dropwise under stirring at 0° C. The resulting mixture was stirred at ambient for 1 h and then cooled down to 0° C. A solution of the compound from Step 1 in anhydrous acetonitrile (2 mL) was added to the reaction and refluxed overnight. The reaction was cooled, poured over ice and filtered. The filtrate was neutralized with 10% sodium carbonate solution and extracted with dichloromethane. The organic phase was dried over sodium sulfate and concentrated in vacuo. The crude material was purified by column chromatography on silica gel using 5% EtOAc in hexanes.

MS: m/e 249/251 (M+1)⁺
Step 3: Preparation of

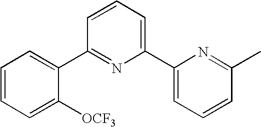

To a mixture of the bromo compound, from Step 2, (0.067 g) and 2-trifluoromethoxyphenyl boronic acid (0.167 g), anhydrous toluene (0.5 mL) and potassium fluoride (0.031 g) were added triphenylphosphine (0.007 g) and palladium acetate (0.003 g) under N₂. The reaction was refluxed for 3 h, cooled and partitioned between EtOAc and water. The organic layer was dried over sodium sulfate and concentrated in vacuo. The crude material obtained was purified by column chromatography on silica gel using a gradient of 12-15% EtOAc in hexanes to yield the pure product.

MS: m/e 331 (M+1)⁺

### EXAMPLE 227

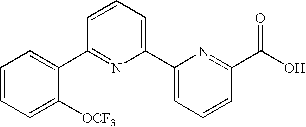

A solution of the methyl pyridine (from Step 3 of Example 226) (0.068 g) in anhydrous pyridine (3 mL) was treated with selenium dioxide (0.4 g). The reaction was refluxed overnight. The reaction was cooled, filtered through Celite and concentrated. The residue dissolved EtOAc, washed with 1 N HCl and water. The organic phase was dried over sodium sulfate and concentrated. The product obtained was carried forward to the next step. MS: m/e 361 (M+1)⁺

### EXAMPLE 228

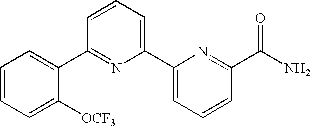

The titled compound was prepared from the acid obtained in Example 227 using the procedure described in Example 216. The crude material was purified by column chromatography on silica gel using 50% EtOAc in hexanes to give the pure amide.

¹H NMR (CDCl₃): 5.88 (s, 1H), 7.44 (d, J=7.6 Hz, 1H), 7.47-7.55 (m, 2H), 7.80 (d, J=7.8 Hz, 1H), 7.96-8.07 (m, 4H), 8.30 (d, J=7.8 Hz, 1H), 8.44 (d, J=8.0 Hz, 1H), 8.75 (d, J=8.0 Hz, 1) MS: m/e 360 (M+1)⁺

### EXAMPLE 229

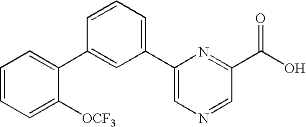 Step 1: Preparation of

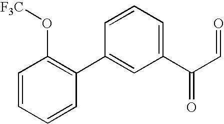

A mixture of selenium dioxide (1.50 g), dioxane (6 mL) and deionized water (0.25 mL) was stirred at 50° C. for 15 minutes to dissolve the selenium dioxide, and then the methyl ketone (from Example 217, Step 4) (3.1 g) was added in one portion to the reaction and refluxed for six hours. The reaction was cooled and filtered. The filtrate was concentrated in vacuo and the residue (yellow) was diluted in 50% EtOAc in hexanes and washed with saturated sodium thiosulfate solution until the organic layer was clear. The organic phase was dried over sodium sulfate and concentrated. The crude keto-aldehyde was used in the next step without further purification.

Step 2:

To a solution of the keto-aldehyde (from Step 1) (2.8 g) in anhydrous methanol (3.1 mL) at −30° C. was added a pre-cooled solution of L-alaninamide hydrochloride (1.20 g) in anhydrous methanol (6.2 ml). A 2M NaOH solution (6.2 ml) was then added dropwise, and the mixture was stirred at 0° C. for 2 h and then 2 h at room temperature. The reaction was quenched with 10 ml of 1N HCl, then neutralized with ˜1 g of solid sodium bicarbonate. The solvent was removed in vacuo and the residue was extracted with EtOAc. The organic phase was washed with water, dried over sodium sulfate and concentrated to give a mixture of regioisomers of pyrazinones that were not separated and carried to the next step. MS: m/e 347 (M+1)⁺

Step 3:

A mixture of the pyrazinone isomers from Step 2 (1.75 g) and POCl₃ (8 mL) were placed in sealed tube and heated to 170° C. for 18 hours. The reaction was concentrated in vacuo and the residue was dissolved in EtOAc. The organic phase was washed with water and saturated sodium bicarbonate solution, then dried over sodium sulfate. The regioisomers were separated by column chromatography on silica gel using a gradient of 5-6% EtOAc in hexanes. The less polar isomer was then taken forward to Step 4 described below. MS: m/e 365 (M+1)⁺

Step 4:

To a solution of the chloropyrazine (from Step 3) (0.31 g) in EtOH (3 mL) were added sodium acetate (77 mg) and 10% (w/w) palladium on carbon (0.1 g). The reaction was shaken under 45 pounds of hydrogen gas for four hours. After that period, the reaction aws filtered through a pad of Celite and the filtrate was concentrated in vacuo. The crude product was purified by column chromatography on silica gel using 15% EtOAc in hexanes to give the titled methyl pyrazine compound.

MS: m/e 331 (M+1)⁺

### EXAMPLE 230

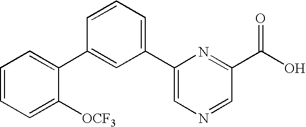

To a solution of the methyl pyrazine (from Example 229 Step 4) (0.051 g) in anhydrous pyridine (0.3 mL) was added a solution of nBu₄N⁺MnO4⁻ (0.11 g) in pyridine (0.3 mL) slowly and the reaction was stirred at room temperature for 30 min. and then at 65° C. overnight. Two additional equivalents of tetrabutylammonium permanganate were added the following morning and the reaction was heated for two more hours. The reaction was allowed to cool to room temperature at which point it was quenched with saturated sodium thiosulfate sulfate. The aqueous layer was acidified to pH=1 with 1 N HCl. The aqueous layer was subsequently extracted with two portions of EtOAc. The organics were further washed with 1 N HCl and dried over sodium sulfate. The organic material was concentrated via rotary evaporator. No further purification was attempted.

MS: m/e 361 (M+1)⁺

### EXAMPLE 231

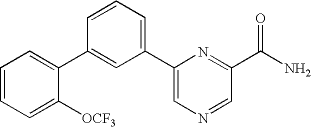

The acid (54 mg) (from Example 230) was dissolved in 200 ul of anhydrous DMF and treated with carbonyl diimidazole (49 mg) at room temperature for 1 hour. Then, solid ammonium acetate (46 mg) was added and the reaction was allowed to continue overnight. The reaction was quenched with ˜4ml of H₂O and the aqueous layer extracted with 2×4 ml portions of EtOAc. The organics were dried over sodium sulfate and concentrated on the rotary evaporator. The crude material was purified by column chromatography on silica gel using 50% EtOAc in hexanes to give the pure amide.

¹H NMR (CDCl₃): 6.06 (s, 1H), 7.42-7.51 (m, 3H), 7.56 (d, J=7.4 Hz, 1H), 7.66-7.70 (m, 2H), 7.82 (s, 1H), 7.95-8.10 (t, 1H), 8.20 (s, 1H), 9.29 (s, 1H), 9.45 (s, 1H). MS: m/e 360 (M+1)⁺

TABLE 8

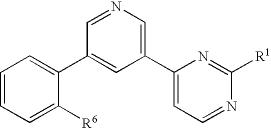

| EXAMPLE # | R⁶ | R¹ | MS (m/e, M + 1) |
|---|---|---|---|
| 232 | OCF₃ | CH₃ | 332 |
| 233 | OCF₃ | COOH | 362 |
| 234 | OCF₃ | COOCH₃ | 376 |
| 235 | OCF₃ | CONH₂ | 361 |

TABLE 9

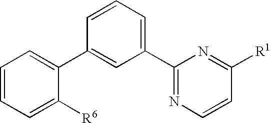

| EXAMPLE # | R⁶ | R¹ | MS (m/e, M + 1) |
|---|---|---|---|
| 236 | OCF₃ | CH₃ | 331 |
| 237 | OCF₃ | COOH | 361 |
| 238 | OCF₃ | CONH₂ | 360 |
| 239 | CF₃ | CH₃ | 315 |
| 240 | CF₃ | COOH | 345 |
| 241 | CF₃ | CONH₂ | 344 |

Further examples of pyrazines compounds prepared are listed below.

TABLE 10

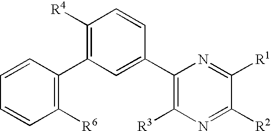

| | | | | | | MS: |
|---|---|---|---|---|---|---|
| EX. | | | | | | m/e |
| # | R⁶ | R⁴ | R³ | R² | R¹ | (M + 1) |
| 242 | OCF₃ | H | H | H | 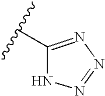 | 385 |
| 243 | OCF₃ | H | H | H | 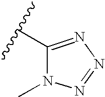 | 399 |
| 244 | OCF₃ | H | H | H | 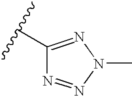 | 399 |
| 245 | OCF₃ | H | H | H | 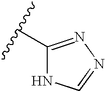 | 384 |
| 246 | OCF₃ | H | H | H | 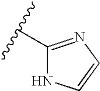 | 383 |
| 247 | OCF₃ | H | H | H | 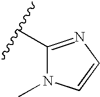 | 397 |
| 248 | OCF₃ | H | H | H | —CH₂CH₂CONH₂ | 388 |
| 249 | OCF₃ | H | H | H | —CH₂CONH₂ | 374 |
| 250 | OCF₃ | H | H | H | —CH₂CN | 356 |
| 251 | OCF₃ | H | H | H | —SO₂NHtBu | 452 |
| 252 | OCF₃ | H | H | H | —SO₂NH₂ | 396 |
| 253 | OCF₃ | H | H | H | —SO₂NHMe | 410 |
| 254 | OCF₃ | H | H | H | —CH₂OH | 347 |
| 255 | OCF₃ | H | H | H | —CH(Me)OH | 361 |
| 256 | OCF₃ | H | H | H | —CH₂NHCOCH₃ | 388 |
| 257 | OCF₃ | H | H | H | —CH₂OSO₂NH₂ | 426 |
| 258 | OCF₃ | H | H | H | —NHCH₃ | 346 |
| 259 | OCF₃ | H | H | H | —NH—CH(CH₃)₂ | 374 |
| 260 | OCF₃ | H | H | H | NH₂ | 332 |
| 261 | OCF₃ | H | H | Cl | CONH₂ | 394 |
| 262 | OCF₃ | H | H | CONH₂ | Cl | 394 |
| 263 | OCF₃ | H | H | H | CONHNH₂ | 375 |
| 264 | OCF₃ | H | H | H | NHSO₂CH₃ | 410 |
| 265 | OCF₃ | H | NH₂ | NH₂ | CONH₂ | 391 |
| 266 | OCF₃ | F | H | H | CONH₂ | 379 |
| 267 | OCF₃ | H | H | CH₃ | OCON(CH₃)₂ | 418 |
| 268 | OCF₃ | H | H | OCON(CH₃)₂ | CH₃ | 418 |
| 269 | OCF₃ | H | H | CONH₂ | OCH₃ | 391 |
| 270 | OCF₃ | H | H | CH₃ | O(CH₂)₂N(CH₃)₂ | 418 |
| 271 | OCF₃ | H | H | O(CH₂)₂N(CH₃)₂ | CH₃ | 418 |
| 272 | OCF₃ | H | H | CH₃ | NHCH₃ | 360 |
| 273 | OCF₃ | H | H | OCH₃ | CONH₂ | 391 |
| 274 | OCF₃ | H | H | Cl | CH₃ | 365 |
| 275 | OCF₃ | H | H | CH₃ | H | 331 |
| 276 | OCF₃ | H | H | H | CH₃ | 331 |
| 277 | OCF₃ | H | H | CONH₂ | H | 360 |
| 278 | OCF₃ | F | H | CONH₂ | H | 378 |
| 279 | OCF₃ | H | H | H | SCH₃ | 363 |
| 280 | OCF₃ | H | H | H | S(O)CH₃ | 379 |
| 281 | OCF₃ | H | H | H | SO₂CH₃ | 395 |
| 282 | OCF₃ | F | H | H | COOH | 379 |
| 283 | OCF₃ | H | H | H | CHO | 345 |
| 284 | OCF₃ | H | H | H | COCH₃ | 359 |
| 285 | OCF₃ | H | H | H | CN | 342 |
| 286 | OCF₃ | H | H | H | H | 316 |
| 287 | OCF₃ | H | H | H | 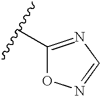 | 385 |
| 288 | OCF₃ | H | H | H | CH(OH)CF₃ | 414 |
| 289 | OCF₃ | H | H | CH(OH)CF₃ | H | 414 |
| 290 | OCF₃ | H | H | CONH₂ | OH | 376 |
| 291 | OCF₃ | H | H | CH₃ | CONH—tBu | 415 |
| 292 | OCF₃ | H | H | H | COCF₃ | 412 |
| 293 | OCF₃ | H | H | H | —OCH₂SO₂NH₂ | 426 |
| 294 | OCF₃ | H | H | H | —CH═CHCO₂CH₃ | 401 |
| 295 | OCF₃ | H | H | H | —CH(NH₂)CH₂CONH₂ | 403 |
| 296 | OCF₃ | H | H | CONH₂ | OCH₃ | 391 |
| 297 | OCF₃ | H | H | H | —CONHCH(CH₃)CONH₂ | 431 |
| 298 | OCF₃ | H | H | H | —CON(CH₃)₂ | 388 |
| 299 | OCF₃ | H | H | H | —O(CH₂)₂N(CH₃)₂ | 404 |
| 300 | OCF₃ | H | H | H | —CH₂NHCOCH₃ | 388 |
| 301 | CF₃ | H | H | H | COOCH₃ | 359 |
| 302 | OCF₃ | H | H | H | S—COCH₃ | 375 |
| 303 | CF₃ | H | H | H | CONH₂ | 344 |
| 304 | OPh | H | H | H | CONH₂ | 368 |
| 305 | OCF₃ | H | H | H | CONHCH₃ | 374 |
| 306 | OCF₃ | H | H | NH₂ | NHCH₃ | 361 |
| 307 | OCF₃ | H | H | NH₂ | COOPr | 403 |
| 308 | Cl | H | H | H | COOCH₃ | 324 |
| 309 | OCF₃ | H | H | NH₂ | CONH₂ | 373 |
| 310 | Cl | H | H | H | CONH₂ | 310 |
| 311 | OCF₃ | H | H | H | CSNH₂ | 376 |
| 312 | OCF₃ | H | H | CH₃ | CONH₂ | 374 |
| 313 | OCF₃ | H | H | OCH₃ | CONH₂ | 390 |
| 314 | OCF₃ | H | H | H | NHCOCH₃ | 374 |
| 315 | OCF₃ | H | H | H | N(COCH₃)₂ | 416 |
| 316 | OCF₃ | H | H | CH₃ | COOH | 375 |
| 317 | OCF₃ | H | H | CONH₂ | CONH₂ | 403 |
| 318 | OCF₃ | H | H | CH(CH₃)₂ | CONH₂ | 402 |
| 319 | OCF₃ | H | H | CONH₂ | CH(CH₃)₂ | 402 |
| 320 | OCF₃ | H | H | CH(CH₃)₂ | CONHC(═NH)NH₂ | 402 |
| 321 | OCF₃ | H | H | CH(CH₃)₂ | CONHOH | 376 |
| 322 | OCF₃ | H | H | H | NHCONH₂ | 374 |
| 323 | OCF₃ | H | CH₃ | H | CONH₂ | 373 |
| 324 | OCF₃ | H | CH₃ | CONH₂ | H | 373 |
| 325 | OCF₃ | H | H | H | NHCH₂CONH₂ | 388 |
| 326 | OCF₃ | H | H | H | NHC(═NH)NH₂ | 374 |
| 327 | OCF₃ | H | H | H | C(═NH)NH₂ | 359 |
| 328 | CF₃ | H | H | H | COOH | 344 |
| 329 | OCF₃ | H | Cl | H | CONH₂ | 394 |
| 330 | OCF₃ | H | CH₃ | COOH | H | 374 |
| 331 | OCF₃ | H | CH₃ | H | COOH | 374 |
| 332 | OCF₃ | H | NH₂ | H | CONH₂ | 375 |
| 333 | OCF₃ | H | NH₂ | H | COOH | 376 |
| 334 | OCF₃ | H | Cl | H | COOH | 395 |
| 335 | OCF₃ | H | NH₂ | CONH₂ | H | 375 |
| 336 | OCF₃ | H | CONH₂ | H | CONH₂ | 403 |
| 337 | OCH₂CF₃ | H | H | CH₃ | Cl | 379 |
| 338 | OCH₂CF₃ | H | H | Cl | CH₃ | 379 |
| 339 | OCH₂CF₃ | H | H | H | CH₃ | 345 |
| 340 | OCH₂CF₃ | H | H | CH₃ | H | 345 |
| 341 | OCH₂CF₃ | H | H | H | CONH₂ | 374 |
| 342 | OCH₂CF₃ | H | H | CONH₂ | H | 374 |
| 343 | OCH₂CF₃ | H | H | H | H | 331 |
| 344 | OCH₂CF₃ | H | H | H | COOH | 375 |
| 345 | 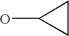 | H | H | H | COOCH₃ | 347 |
| 346 | 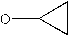 | H | H | H | CONH₂ | 332 |
| 347 | OCF₃ | H | H | H | CONHC(CH₃)₂CONH₂ | 445 |
| 348 | OCF₃ | H | H | H | CH(OH)CH₃ | 361 |
| 349 | OCF₃ | H | H | H | NHSO₂NH₂ | 411 |
| 350 | OCF₃ | H | H | H | N(CH₃)CONH₂ | 389 |
| 351 | OCF₃ | H | H | CH₃ | N(CH₃)CONH₂ | 403 |
| 352 | OCF₃ | H | H | N(CH₃)CONH₂ | CH₃ | 403 |

TABLE 11

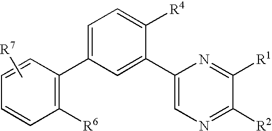

| | | | | | | MS: |
|---|---|---|---|---|---|---|
| EX. | | | | | | m/e |
| # | R⁶ | R⁷ | R⁴ | R² | R¹ | (M + 1) |
| 353 | CF₃ | 5-F | H | H | CONH₂ | 362 |
| 354 | CF₃ | 5-F | H | CONH₂ | H | 362 |
| 355 | CF₃ | 4-CF₃ | H | H | CONH₂ | 412 |
| 356 | CF₃ | 4-CF₃ | H | CONH₂ | H | 412 |
| 357 | OCF₃ | H | F | H | CONH₂ | 378 |
| 358 | OCF₃ | H | F | CONH₂ | H | 378 |
| 359 | CF₃ | 4-CF₃ | H | H | H | 369 |
| 360 | Cl | 3-Cl | H | H | COOCH₃ | 358 |
| 361 | Cl | 4-Cl | H | H | COOCH₃ | 358 |
| 362 | Cl | 3-Cl | H | H | CONH₂ | 344 |
| 363 | Cl | 4-Cl | H | H | CONH₂ | 344 |
| 364 | Cl | 6-Cl | H | H | CONH₂ | 344 |

### EXAMPLE 365

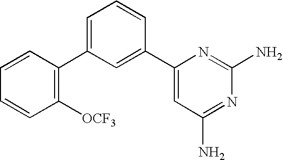

A mixture of 2-trifluoromethoxyphenyl boronic acid obtained from Step 1 of Example 36 (0.41 g, 2 mMol) and 3-bromophenyl boronic acid (0.4 g, 2 mMol) in n-propanol (5 ml) was placed in a microwave reaction tube and stirred at room temperature under N₂ for 15 min. To the resulting solution were then added Ph₃P (0.025 g) and Pd(OAc)₂ (0.005 g) followed by 2M Na₂CO₃ (1.2 mL) and water (0.7 mL). The tube was sealed and the tube was heated in Smith Creator Personal Chemistry Microwave Instrument at 150° C. for 900 sec. The reaction was cooled and diluted with water. The mixture was acidified with 1N HCl and extracted with EtOAc. The organic phase was washed with water, dried and concentrated in vacuo. The LCMS indicated the desired biphenyl boronic acid, which without further purification was dissolved in a mixture of toluene (1.5 mL) and n-propanol (1.5 mL). The solution was placed in a microwave reaction tube and was added Ph₃P (0.050 g) and Pd(OAc)₂ (0.005 g) followed by 2M Na₂CO₃ (1.2 rnL) and water (0.6 mL). The sealed reaction tube was heated in Smith Creator Personal Chemistry Microwave Instrument at 150° C. for 1200 sec. The reaction was cooled diluted with water and extracted with EtOAc. The organic phase was washed with water, dried and concentrated in vacuo. The crude product was purified by radial chromatography using chloroform-methanol-ammonia (10:1:0.1) as the eluent to give the desired product.

¹HNMR (CDCL₃) (δ, ppm): 8.0 (s, 1H), 7.94 (d, J=7.6 Hz ,1H), 7.5-7.6 (m, 3H), 7.36-7.44 (m, 3H), 6.35 (s, 1H). MS (ESI): M/E 347 (M+1)⁺

What is claimed is:

1. A compound represented by Formula (I) or (II): 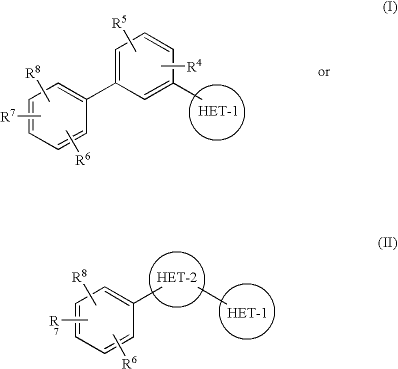
or a pharmaceutically acceptable salt thereof, wherein
HET-1 is one of the following heterocycles: 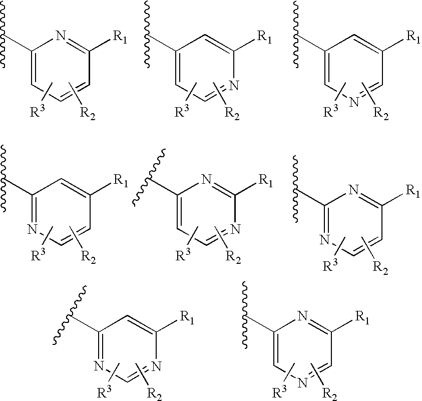
HET-2 is one of the following heterocycles: 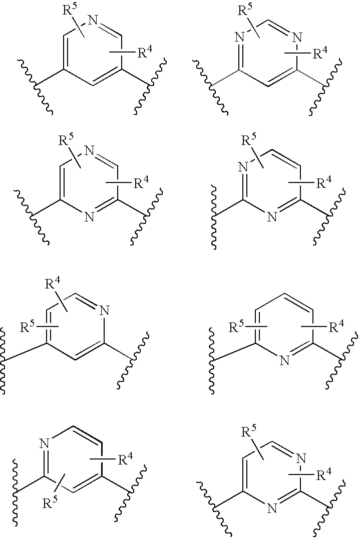
R¹ is:
(a) H;
(b) C₁-C₆-alkyl, C₂-C₄-alkenyl, C₂-C₄-alkynyl, C₁-C₆-cycloalkyl, or C₁-C₄-alkyl-[C₁-C₆-cycloalkyl], any of which is optionally substituted with one or more of the following substituents: F, CF₃, OH, O—(C₁-C₄)alkyl, S(O)₀₋₂—(C₁-C₄)alkyl, O—CONR^{a}R^{b}, NR^{a}R^{b}, N(R^{a})CONR^{a}R^{b}, COO—(C₁-C₄)alkyl, COOH, CN, CONR^{a}R^{b}, SO₂NR^{a}R^{b}, N(R^{a})SO₂NR^{a}R^{b}, —C(═NH)NH₂, tetrazolyl, triazolyl, imidazolyl, oxazolyl, oxadiazolyl, isooxazolyl, thiazolyl, furyl, thienyl, pyrazolyl, pyrrolyl, pyridyl, pyrimidinyl, pyrazinyl, phenyl, piperidinyl, morpholinyl, pyrrolidinyl or piperazinyl;
(c) —O—C₁-C₆-alkyl, —O—C₁-C₆-cycloalkyl, —S—C₁-C₆-alkyl or —S—C₁-C₆-cycloalkyl, any of which is optionally substituted with one or more of the following substituents: F, CF₃, OH, O—(C₁-C₄)alkyl, S(O)₀₋₂—(C₁-C₄)alkyl, O—CONR^{a}R^{b}, NR^{a}R^{b}, N(R^{a})CONR^{a}R^{b}, COO—(C₁-C₄)alkyl, COOH, CN, CONR^{a}R^{b}, SO₂NR^{a}R^{b}, N(R^{a})SO₂NR^{a}R^{b}, —C(═NH)NH₂, tetrazolyl, triazolyl, imidazolyl, oxazolyl, oxadiazolyl, isooxazolyl, thiazolyl, furyl, thienyl, pyrazolyl, pyrrolyl, pyridyl, pyrimidinyl, pyrazinyl, phenyl, piperidinyl, morpholinyl, pyrrolidinyl or piperazinyl;
(d) —CO—C₄-alkyl-C₁-C₄-perfluoroalkyl, or —O—C₀-C₄-alkyl-C₁-C₄-perfluoroalkyl;
(e) —OH;
(f) —O-aryl, or —O—C₁-C₄-alkyl-aryl, wherein aryl is phenyl, pyridyl, pyrimidinyl, furyl, thienyl, pyrrolyl, triazolyl, pyrazolyl, thiazolyl, isoxazolyl, oxazolyl, or oxadiazolyl, any aryl of which is optionally substituted with 1-3 substituents selected from i) F, Cl, Br, I, ii) —CN, iii) —NO₂, iv) —C(═O)(R^{a}), v) —OR^{a}, vi) —NR^{a}R^{b}, vii) —C₀₋₄alkyl—CO—OR^{a}, viii) —(C₀₋₄alkyl)—NH—CO—OR^{a}, ix) —(C₀₋₄alkyl)—CO—N(R^{a})(R^{b}), x) —S(O)₀₋₂R^{a}, xi) —SO₂N(R^{a})(R^{b}), xii) —NR^{a}SO₂R^{a}, xiii) —C₁₋₁₀alkyl, and xiv) —C₁₋₁₀alkyl, wherein one or more of the alkyl carbons can be replaced by a —NR^{a}—, —O—, —S(O)₁₋₂—, —O—C(O)—, —C(O)—O—, —C(O)—N(R^{a})—, —N(R^{a})—C(O)—, —N(R^{a})—C(O)—N(R^{a})—, —C(O)—, —CH(OH)—, —C═C—, or —C≡C—;
(g) —OCON(R^{a})(R^{b}), or —OSO₂N(R^{a})(R^{b});
(h) —SH, or —SCON(R^{a})(R^{b});
(i) NO₂;
(j) NR^{a}R^{b}, —N(COR^{a})R^{b}, —N(SO₂R^{a})R^{b}, —N(R^{a})CON(R^{a})₂, —N(R^{a})CONH₂, —N(OR^{a})CONR^{a}R^{b}, —N(R^{a})CON(R^{a})₂, or —N(R^{a})SO₂N(R^{a})₂;
(k) —CH(OR^{a})R^{a}, —C(OR^{b})CF₃, —CH(NHR^{b})R^{a}, —C(═O)R^{a}, C(═O)CF₃, —SOCH₃, —SO₂CH₃, —N(R^{a})SO₂R^{a}, COOR^{a}, CN, CONR^{a}R^{b}, —COCONR^{a}R^{b}, —SO₂NR^{a}R^{b}, —CH₂O—SO₂NR^{a}R^{b}, SO₂N(R^{a})OR^{a}, —C(═NH)NH₂, —CR^{a}═N—OR^{a}, CH═CHCONR^{a}R^{b}, CONR^{a}, CONHR^{a};
(l) —CONR^{a}(CH₂)₀₋₂C(R^{a})(R^{b})(CH₂)₀₋₂CONR^{a}R^{b};
(m) tetrazolyl, tetrazolinonyl, triazolyl, triazolinonyl, imidazolyl, imidozolonyl, oxazolyl, oxadiazolyl, isooxazolyl, thiazolyl, furyl, thienyl, pyrazolyl, pyrazolonyl, pyrrolyl, pyridyl, pyirimdinyl, pyrazinyl, or phenyl, any of which is optionally substituted with 1-3 independent substituents selected from i) F, Cl, Br, I, ii) —CN, iii) —NO₂, iv) —C(═O)R^{a}, v) C₁-C₆-alkyl, vi) —O—R^{a}, vii) —NR^{a}R^{b}, viii) —C₀-C₄-alkyl —CO—O R^{a}, ix) —(C₀-C₄-alkyl)—NH—CO—OR^{a}, x) —(C₀-C₄-alkyl)—CO—NR^{a}R^{b}, xi) —S(O)₀₋₂R^{a}, xii) —SO₂NR^{a}R^{b}, xiii) —NHSO₂R^{a}, xiv) —C₁-C₄-perfluoroalkyl, and xv) —O—C₁-C₄-perfluoroalkyl;
(n) —C(R^{a})═C(R^{b})—COOR^{a}, or —C(R^{a})═C(R^{b})—CONR^{a}R^{b};
(o) piperidin-1-yl, morpholin-4-yl, pyrrolidin-1-yl, piperazin-1-yl or 4-susbstituted piperazin-1-yl, any of which is optionally substituted with 1-3 substituents selected from i) —CN, ii) 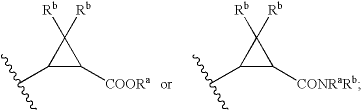
—C(═O)(R^{a}), iii) C₁-C₆-alkyl, iv) —OR^{a}, v) —NR^{a}R^{b}, vi) —C₀-C₄-alkyl-CO—OR^{a}, vii) —(C₀-C₄-alkyl)—NH—CO—OR^{a}, viii) —(C₀-C₄-alkyl)—CON(R^{a})(R^{b}), ix) —SR^{a}, x) —S(O)₀₋₂R^{a}, xi) —SO₂N(R^{a})(R^{b}), xii) —NR^{a}SO₂R^{a} xiii) —C₁-C₄-perfluoroalkyl and xiv) —O—C₁-C₄-perfluoroalkyl;
R^{a} is
(a) H;
(b) C₁-C₄-alkyl, optionally substituted with one or more of the following substituents: F, CF₃, OH, O—(C₁-C₄)alkyl, S(O)₀₋₂—(C₁-C₄)alkyl, —OCONH₂, —OCONH(C₁-C₄alkyl), —OCON(C₁-C₄alkyl)(C₁-C₄alkyl), —OCONH(C₁-C₄alkyl-aryl), —OCON(C₁-C₄alkyl)(C₁-C₄alkyl-aryl), NH₂, NH(C₁-C₄alkyl), N(C₁-C₄alkyl)(C₁-C₄alkyl), NH(C₁-C₄alkyl-aryl), N(C₁-C₄alkyl)(C₁-C₄alkyl-aryl), NHCONH₂, NHCONH(C₁-C₄alkyl), NHCONH(C₁-C₄alkyl-aryl), —NHCON(C₁-C₄alkyl)(C₁-C₄alkyl), NHCON(C₁-C₄alkyl)(C₁-C₄alkyl-aryl), N(C₁-C₄alkyl)CON(C₁-C₄alkyl)(C₁-C₄alkyl), N(C₁-C₄alkyl)CON(C₁-C₄alkyl)(C₁-C₄alkyl-aryl), CO—(C₁-C₄-alkyl), COOH, CN, CONH₂, CONH(C₁-C₄alkyl), CON(C₁-C₄alkyl)(C₁-C₄alkyl), SO₂NH₂, SO₂NH(C₁-C₄alkyl), SO₂NH(C₁-C₄alkyl-aryl), SO₂N(C₁-C₄alkyl)(C₁-C₄alkyl), NHSO₂NH₂, —C(═NH)NH₂, tetrazolyl, triazolyl, imidazolyl, oxazolyl, oxadiazolyl, isooxazolyl, thiazolyl, furyl, thienyl, pyrazolyl, pyrrolyl, pyridyl, pyritridinyl, pyrazinyl, phenyl, piperidinyl, morpholinyl, pyrrolidinyl or piperazinyl;
(c) C₀-C₄-alkyl-(C₁-C₄)-perfluoroalkyl; or
(d) C₁-C₄-alkyl-aryl, wherein aryl is phenyl, pyridyl, pyxrimdinyl, furyl, thienyl, pyrrolyl, triazolyl, pyrazolyl, thiazolyl, isoxazolyl, oxazolyl, or oxadiazolyl, any aryl of which is optionally substituted with 1-3 substituents selected from i) F, Cl, Br, I, ii) —CN, iii) —NO₂, iv) —C(═O)(C₁-C₄-alkyl), v) —O(C₁-C₄-alkyl), vi) —N(C₁-C₄-alkyl)(C₁-C₄-alkyl), vii) —C₁₋₁₀alkyl, and viii) —C₁₋₁₀alkyl, wherein one or more of the alkyl carbons can be replaced by a, —O—, —S(O)₁₋₂—, —O—C(O)—, —C(O)—O—, —C(O)—, —CH(OH)—, —C═C—, or —C≡C—;
R^{b} is
(a) H; or
(b) C₁-C₆-alkyl, optionally substituted with one or more of the following substituents: F, CF₃, OH, O—(C₁-C₄)alkyl, S(O)₀₋₂—(C₁-C₄)alkyl, —OCONH₂, —OCONH(C₁-C₄alkyl), NH₂, NH, NH(C₁-C₄alkyl), N(C₁-C₄alkyl), N(C₁-C₄alkyl)(C₁-C₄alkyl), NHCONH₂, NHCONH(C₁-C₄alkyl), —NHCON(C₁-C₄alkyl)(C₁-C₄alkyl), COO—(C₁-C₄-alkyl), COOH, CN, pyridyl, piperidinyl, pyriridinyl, piperazinyl, CONH₂ or (C₁-C₄alkyl)CONH₂; or
R^{a} and R^{b}, together with the N to which they are attached, can form a 5- or 6-membered ring which optionally contains a heteroatom selected from N, O, and S, and wherein said ring is optionally substituted with 1-3 substituents selected from i) F, Cl, Br, I, ii) —CN, iii) —NO₂, iv) —C(═O)(Ra), v) —OR^{a}, vi) —NR^{a}R^{b}, vii) —C₀₋₄alkyl-CO—OR^{a}, viii) —(C₀₋₄alkyl)—NH—CO—OR^{a}, ix) —(C₀₋₄alkyl)—CO—N(R^{a})(R^{b}), x) —S(O)₀₋₂R^{a}, xi) —SO₂N(R^{a})(R^{b}), xii) —NR^{a}SO₂R^{a}, xiii) —C₁₋₁₀alkyl, and xiv) —O—;
R² and R³ each independently is:
(a) H;
(b) —C₁-C₄-alkyl, or —O—C₁-C₄-alkyl;
(c) —C₀-C₄-alkyl-C₁-C₄-perfluoroalkyl, or —O—C₀-C₄-alkyl-C₁-C₄-perfluoroalkyl; or
(d) CN, N R^{a} R^{b}, NO₂, F, Cl, Br, I, OH, OCONR^{a} R^{b}, O(C₁-C₄-alkyl)CONR^{a} R^{b}, —OSO₂NR^{a}R^{b}, COOR^{a}, or CONR^{a}R^{b};
R⁴ and R⁵ each independently is:
(a) H;
(b) —C₁-C₆-alkyl, —C₂-C₆-alkenyl, —C₂-C₆-alkynyl or —C₁-C₆-cycloalkyl, any of which is optionally substituted with one or more of the following substituents: F, CF₃, —O—(C₁-C₄)alkyl, CN, —N(R^{a})(R^{b}), —N(R^{a})CO—(C₁-C₄)alkyl, COOR^{b}, CON^{a})(R^{b}) and phenyl;
(c) —O—C₀—C₆-alkyl, —O-aryl, or —O—C₁-C₄-alkyl-aryl, wherein aryl is phenyl, pyridyl, pyrimidinyl, furyl, thienyl, pyrrolyl, triazolyl, pyrazolyl, thiazolyl, isoxazolyl, oxazolyl, or oxadiazolyl, any aryl of which is optionally substituted with 1-3 substituents selected from i) F, Cl, Br, I, ii) —CN, iii) —NO₂, iv) —C(═O)(R^{a}), v) —OR^{a}, vi) —NR^{a}R^{b}, vii) —C₀₋₄alkyl-CO—OR^{a}, viii) —(C₀₋₄alkyl)—NH—CO—OR^{a}, ix) —(CO—₄alkyl)—CO—N(R^{a})(R^{b}), x) —S(O)₀₋₂R^{a}, xi) —SO₂N(R^{a})(R^{b}), xii) —NR^{a}SO₂R^{a}, xiii) —C₁₋₁₀alkyl, and xiv) —C₁₋₁₀alkyl, wherein one or more of the alkyl carbons can be replaced by a —NR^{a}, —O—, —S(O)₁₋₂—, —O—C(O)—, —C(O)—O—, —C(O)—N(R^{a})—, N(R^{a})—C(O)—, N(R^{a}) C(O)—N(R^{a})—, —C(O)—, —CH(OH)—, —C═C—, or —C≡C—;
(d) —C₀-C₄-alkyl-C₁-C₄-perfluoroalkyl, or —O—CO—C₄-alkyl-C₁-C₄-perfluoroalkyl; or
(e) CN, NH₂, NO₂, F, Cl, Br, I, OH, OCON(R^{a})(R^{b}) O(C₁-C₄-alkyl)CONR^{a}R^{b}, —OSO₂N(R^{a})(R^{b}), COOR^{b}, CON(R^{a})(R^{b}), or aryl, wherein aryl is phenyl, pyridyl, pyrimidinyl, furyl, thienyl, pyrrolyl, triazolyl, pyrazolyl, thiazolyl, isoxazolyl, oxazolyl, or oxadiazolyl, any aryl of which is optionally substituted with 1-3 substituents selected from i) F, Cl, Br, I, ii) —CN, iii) —NO₂, iv) —C(═O)(R^{a}), v) —OR^{a}, vi) —NR^{a}R^{b}, vii) —C₀₋₄alkyl-CO—OR^{a}, viii) —(C₀₋₄alkyl)—NH—CO—OR^{a}, ix) —(C₀₋₄alkyl)—CO—N(R^{a})(R^{b}), x) —S(O)₀₋₂R^{a}, xi) —SO₂N(R^{a})(R^{b}), xii) —NR^{a}SO₂R^{a}, xiii) —C₁₋₁₀alkyl, and xiv) —C₁₋₁₀alkyl, wherein one or more of the alkyl carbons can be replaced by a —NR^{a}—, —O—, —S(O)₁₋₂—, —O—C(O)—, —C(O)—O—, —C(O)—N(R^{a})—, —N(R^{a})—C(O)—, —N(R^{a})—C(O)—N(R^{a})—, —C(O)—, —CH(OH)—, —C═C—, or —C≡C; and
R⁶, R⁷ and R⁸ each independently is:
(a) H;
(b) C₁-C₆-alkyl, C₂-C₄-alkenyl, C₂-C₄-alkynyl or C₁-C₆-cycloalkyl, any of which is optionally substituted with one or more of the following substituents: F, CF₃, OH, O—(C₁-C₄)alkyl, OCON(R^{a})(R^{b}), NR^{a}R^{b}, COOR^{a}, CN, CONR^{a}R^{b}, N(R^{a})CONR^{a}R^{b}, N(R^{a})SO₂NR^{a}R^{b}, SO₂NR^{a}R^{b}, S(O)₀₋₂(C₁-C₄-alkyl), —C(═NH)NH₂, tetrazolyl, triazolyl, imidazolyl, oxazolyl, oxadiazolyl, isooxazolyl, thiazolyl, furyl, thienyl, pyrazolyl, pyrrolyl, pyridyl, pyrimidinyl, pyrazinyl, phenyl, piperidinyl, morpholinyl, pyrrolidinyl, and piperazinyl;
(c) —O—C₁-C₆-alkyl, —O—C₁-C₆-cycloalkyl, —S—C₁-C₆-alkyl or —S—C₁-C₆-cycloalkyl, any of which is optionally substituted with one or more of the following substituents: F, CF₃, OH, O—(C₁-C₄)alkyl, NH₂, NH(C₁-C₄-alkyl), N(C₁-C₄-alkyl)₂, COOH, CN, CONH₂, CONH(C₁-C₄-alkyl), CONH(C₁-C₄-alkyl)₂, SO₂NH₂, SO₂NH(C₁-C₄-alkyl), tetrazolyl, triazolyl, imidazolyl, oxazolyl, oxadiazolyl, isooxazolyl, thiazolyl, furyl, thienyl, pyrazolyl, pyrrolyl, pyridyl, pyrimidinyl, pyrazinyl, phenyl, piperidinyl, morpholinyl, pyrrolidinyl, or piperazinyl;
(d) —C₀-C₄-alkyl-C₁-C₄-perfluoroalkyl, or —O—C₀-C₄-alkyl-C₁-C₄-perfluoroalkyl;
(e) —O-aryl, or —O—C₁-C₄-alkyl-aryl, wherein aryl is phenyl, pyridyl, pyrimidinyl, furyl, thienyl, pyrrolyl, triazolyl, pyrazolyl, thiazolyl, isoxazolyl, oxazolyl, or oxadiazolyl, any aryl of which is optionally substituted with 1-3 substituents selected from i) F, Cl, Br, I, ii) —CN, iii) —NO₂, iv) —C(═O)(R^{a}), v) —OR^{a}, vi) —NR^{a}R^{b}, vii) —C₀₋₄alkyl-CO—OR^{a}, viii) —(C₀₋₄alkyl)—NH—CO—OR^{a}, ix) —(C₀₋₄alkyl)—CO—N(R^{a})(R^{b}), x) —S(O)₀₋₂R^{a}, xi) —SO₂N(R^{a})(R^{b}), xii) —NR^{a}SO₂R^{a}, xiii) —C₁₋₁₀alkyl, and xiv) —C₁₋₁₀alkyl, wherein one or more of the alkyl carbons can be replaced by a —NR^{a}—, —O—, —S(O)₁₋₂—, —O—C(O)—, —C(O)—O—, —C(O)—N(R^{a})—, —N(R^{a})—C(O)—, —N(R^{a})—C(O)—N(R^{a})—, —C(O)—, —CH(OH)—, —C═C—, or —C≡C; or
(f) CN, N(R^{a})(R^{b}), NO₂, F, Cl, Br, I, —OR^{a}, —SR^{a}, —OCON(R^{a})(R^{b}), —OSO₂N(R^{a})(R^{b}), COOR^{b}, CON(R^{a})(R^{b}), —N(R^{a})CON(R^{a})(R^{b}), —N(R^{a})SO₂N(R^{a})(R^{b}), —C(OR^{b})R^{a}, C(OR^{a})CF₃, —C(NHR^{a})CF₃, —C(═O)R^{a}, C(═O)CF₃, —SOCH₃, —SO₂CH₃, —NHSO₂(C₁₋₆-alkyl), —NHSO₂-aryl, SO₂N(R^{a})(R^{b}), —CH₂OSO₂N(R^{a})(R^{b}), SO₂N(R^{b})—OR^{a}, —C(═NH)NH₂, —CRₐ═N—ORₐ, CH═CH or aryl, wherein aryl is phenyl, pyridyl, pyrimidinyl, furyl, thienyl, pyrrolyl, triazolyl, pyrazolyl, thiazolyl, isoxazolyl, oxazolyl, or oxadiazolyl, any aryl of which is optionally substituted with 1-3 substituents selected from i) F, Cl, Br, I, ii) —CN, iii) —NO₂, iv) —C(═O)(R^{a}), v) —OR^{a}, vi) —NR^{a}R^{b}, vii) —C₀₋₄alkyl-CO—OR^{a}, viii) —(C₀₋₄alkyl)—NH—CO—OR^{a}, ix) —(C₀₋₄alkyl)—CO—N(R^{a})(R^{b}), x) —S(O)₀₋₂R^{a}, xi) —SO₂N(R^{a})(R^{b}), xii) —NR^{a}SO₂R^{a}, xiii) —C₁₋₁₀alkyl, and xiv) —C₁₋₁₀alkyl, wherein one or more of the alkyl carbons can be replaced by a —NR^{a}—, —O—, —S(O)₁₋₂—, —O—C(O)—, —C(O)—O—, —C(O)—N(R^{a})—, —N(R^{a})—C(O)—, —N(R^{a})—C(O)—N(R^{a})—, —C(O)—, —CH(OH)—, —C═C—, or —C≡C; or when R⁶ and R⁷ are present on adjacent carbon atoms, R⁶ and R⁷, together with the benzene ring to which they are attached, can form a bicyclic aromatic ring selected from naphthyl, indolyl, quinolinyl, isoquinolinyl, quinoxalinyl. benzofuryl, benzothienyl, benzoxazolyl, benzothiazolyl, and benzimidazolyl, any of which is optionally substituted with 1-4 independent substituents selected from i) halogen, ii) —CN, iii) —NO₂, iv) —CHO, v) —O—C₁₋₄alkyl, vi) —N(C₀₋₄alkyl)(C₀₋₄alkyl), vii) —C₀₋₄alkyl-CO—O(C₀₋₄alkyl), viii) —(C₀₋₄alkyl)—NH—CO—O(C₀₋₄alkyl), ix) —(C₀₋₄alkyl)—CO—N(C₀₋₄alkyl)(C₀₋₄alkyl), x) —S(C₀₋₄alkyl), xi) —S(O)(C₁₋₄alkyl), xii) —SO₂(C₀₋₄alkyl), xiii) —SO₂N(C₀₋₄alkyl)(C₀₋₄alkyl), xiv) —NHSO₂(C₀₋₄alkyl)(C₀₋₄alkyl), xv) —C₁₋₁₀alkyl and xvi) —C₁₋₁₀alkyl in which one or more of the carbons can be replaced by a —N(C₀₋₆alkyl)—, —O—, —S(O)₁₋₂—, —O—C(O)—, —C(O)—O—, —C(O)—N(C₀₋₆alkyl)—, —N(C₀₋₆alkyl)—C(O)—, —N(C₀₋₆alkyl)—C(O)—N(C₀₋₆alkyl)—, —C(O)—, —CH(OH), —C═C—, or —C≡C—.

2. The compound according to claim 1 represented by Formula (I), or a pharmaceutically acceptable salt thereof.

3. The compound according to claim 2, or a pharmaceutically acceptable salt thereof, wherein
HET-1 is

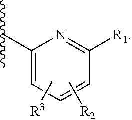

4. The compound according to claim 2, or a pharmaceutically acceptable salt thereof, wherein
HET-1 is

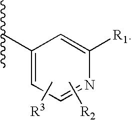

5. The compound according to claim 2, or a pharmaceutically acceptable salt thereof, wherein
HET-1 is

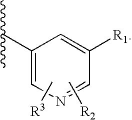

6. The compound according to claim 2, or a pharmaceutically acceptable salt thereof, wherein
HET-1 is

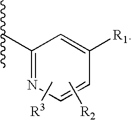

7. The compound according to claim 2, or a pharmaceutically acceptable salt thereof, wherein
HET-1 is

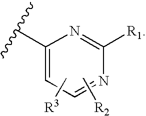

8. The compound according to claim 2, or a pharmaceutically acceptable salt thereof, wherein
HET-1 is

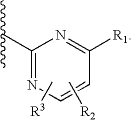

9. The compound according to claim 2, or a pharmaceutically acceptable salt thereof, wherein
HET-1 is

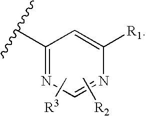

10. The compound according to claim 2, or a pharmaceutically acceptable salt thereof, wherein
HET-1 is

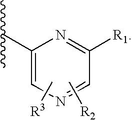

11. The compound according to claim 2, or a pharmaceutically acceptable salt thereof, wherein
R6 is other than H and is attached at the ortho position.

12. The compound according to claim 1 represented by Formula (II), or a pharmaceutically acceptable salt thereof.

13. The compound according to claim 12, or a pharmaceutically acceptable salt thereof, wherein
HET-2 is

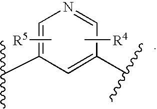

14. The compound according to claim 12, or a pharmaceutically acceptable salt thereof, wherein
HET-2 is

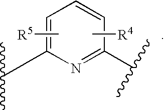

15. The compound according to claim 12, or a pharmaceutically acceptable salt thereof, wherein
HET-2 is

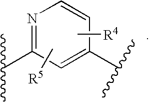

16. The compound according to claim 12, or a pharmaceutically acceptable salt thereof, wherein
HET-2 is

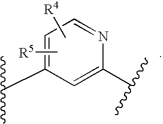

17. The compound according to claim 12, or a pharmaceutically acceptable salt thereof, wherein
HET-2 is

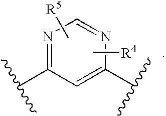

18. The compound according to claim 12, or a pharmaceutically acceptable salt thereof, wherein
HET-2 is

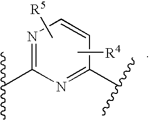

19. The compound according to claim 12, or a pharmaceutically acceptable salt thereof, wherein
HET-2 is

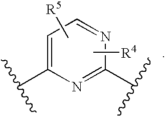

20. The compound according to claim 12, or a pharmaceutically acceptable salt thereof, wherein
HET-2 is

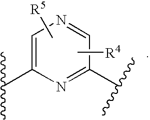

21. The compound according to claim 12, or a pharmaceutically acceptable salt thereof, wherein
HET-1 is 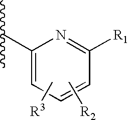
and
HET-2 is

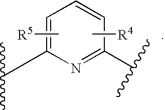

22. The compound according to claim 12, or a pharmaceutically acceptable salt thereof, wherein
HET-1 is 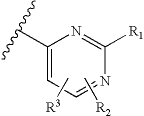
and
HET-2 is

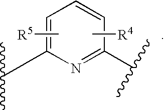

23. The compound according to claim 12, or a pharmaceutically acceptable salt thereof, wherein
HET-1 is 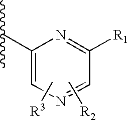
and
HET-2 is

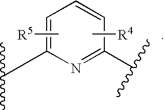

24. The compound according to claim 12, or a pharmaceutically acceptable salt thereof, wherein
HET-1 is

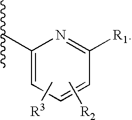

25. The compound according to claim 12, or a pharmaceutically acceptable salt thereof, wherein
HET-1 is

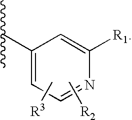

26. The compound according to claim 12, or a pharmaceutically acceptable salt thereof, wherein
HET-1 is

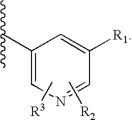

27. The compound according to claim 12, or a pharmaceutically acceptable salt thereof, wherein
HET-1 is

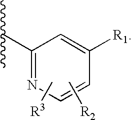

28. The compound according to claim 12, or a pharmaceutically acceptable salt thereof, wherein
HET-1 is

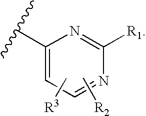

29. The compound according to claim 12, or a pharmaceutically acceptable salt thereof, wherein
HET-1 is

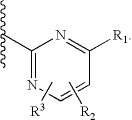

30. The compound according to claim 12, or a pharmaceutically acceptable salt thereof, wherein
HET-1 is

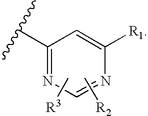

31. The compound according to claim 12, or a pharmaceutically acceptable salt thereof, wherein
HET-1 is

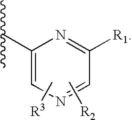

32. A compound represented by 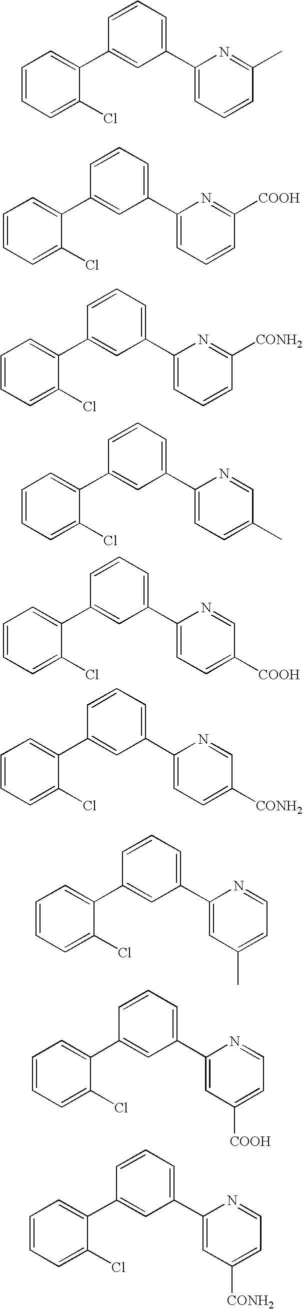 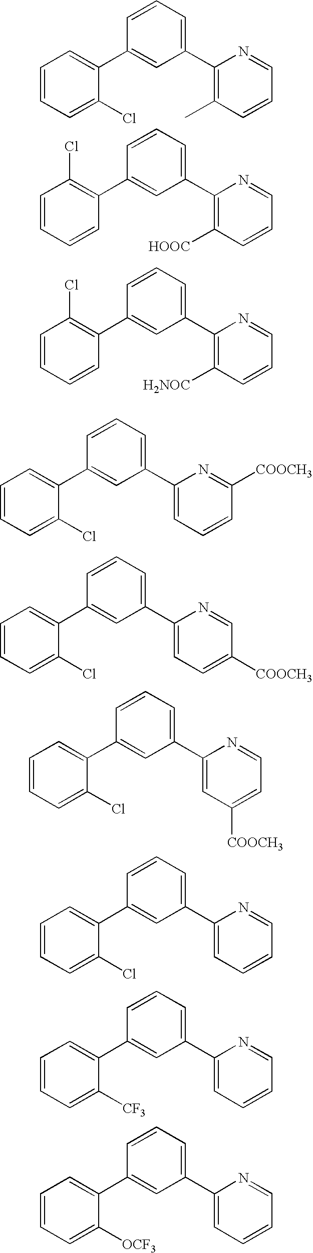 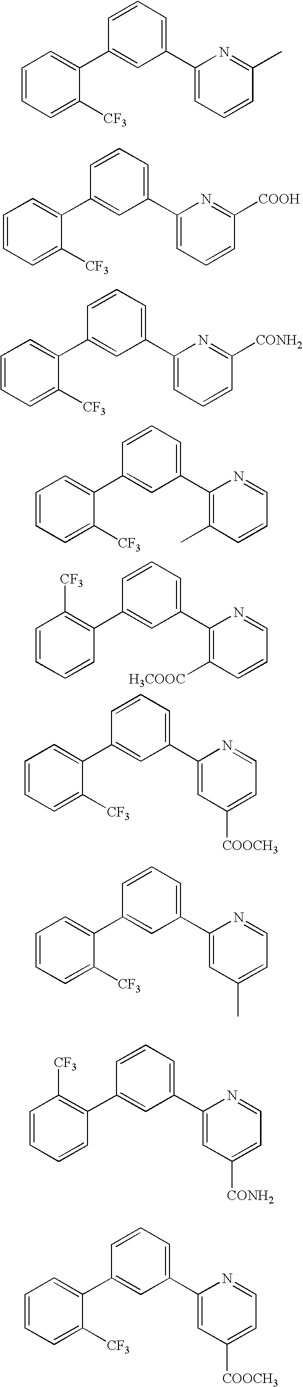 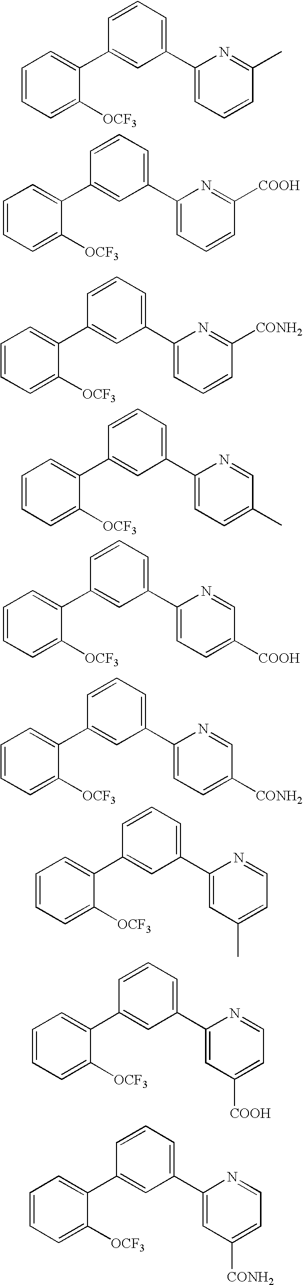 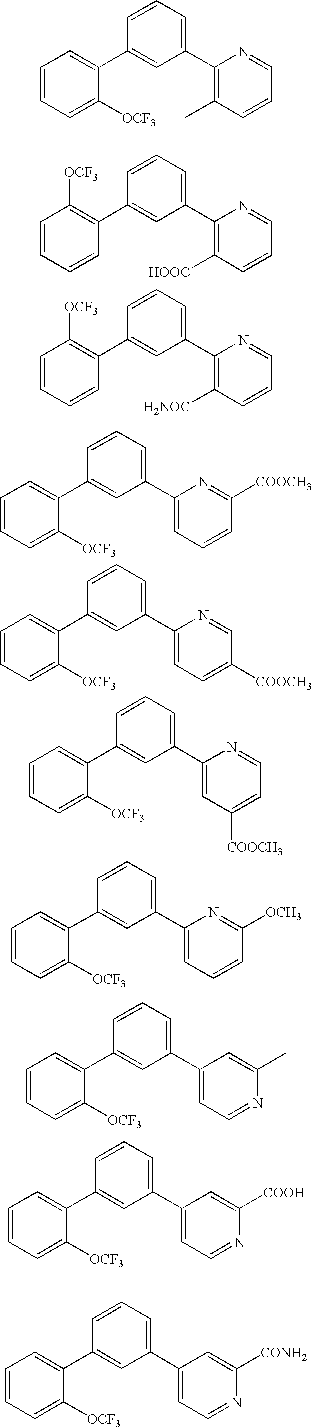 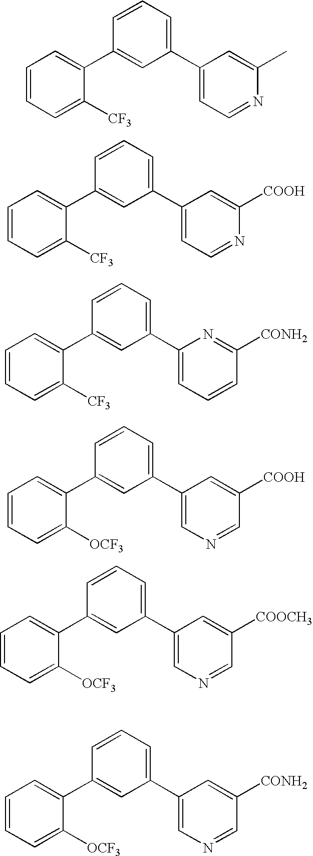
or a pharmaceutically acceptable salt thereof.

33. A compound represented by 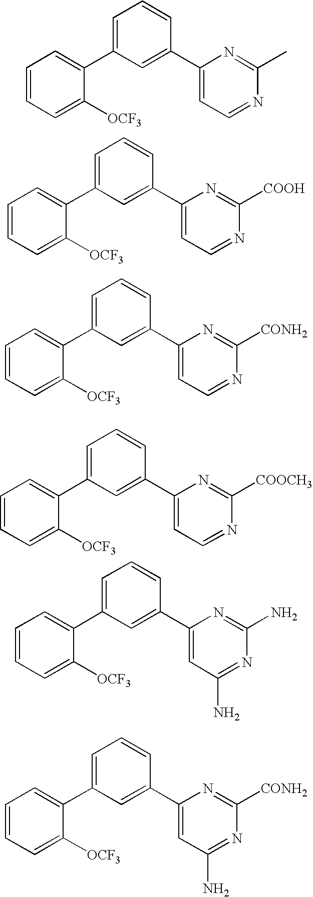
or a pharmaceutically acceptable salt thereof.

34. The compound of claim 1 represented by

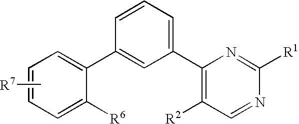

| R⁶ | R⁷ | R² | R¹ |
|---|---|---|---|
| OCF₃ | H | H | H |
| OCF₃ | H | H | 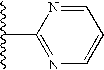 |
| OCF₃ | H | H | —SCH₃ |
| OCF₃ | H | H | —SO₂CH₃ |
| OCF₃ | H | H | —SOCH₃ |
| OCF₃ | H | H | NH₂ |
| OCF₃ | H | H | NHSO₂CH₃ |
| OCF₃ | H | H | N(SO₂CH₃)₂ |
| OCF₃ | H | H | NHCO(CH₃)₃ |
| OCF₃ | H | H | CON(CH₃)OCH₃ |
| OCF₃ | H | H | 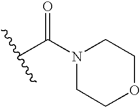 |
| OCF₃ | H | H | CH₃CO |
| OCF₃ | H | H | CONHC(CH₃)₂COOCH₃ |
| OCF₃ | H | H | CONHCH₂CH₂CN |
| OCF₃ | H | H | CONHC(CH₃)₂COOH |
| OCF₃ | H | H | CONHC(CH₃)₂CONH₂ |
| OCF₃ | H | H | CON(CH₂CH₂)₂NH |
| OCF₃ | H | H | 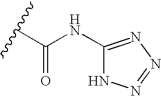 |
| OCH₃ | H | H | CONHC(CH₂)₂COOCH₃ |
| OCF₃ | H | H | CONHC(CH₂)₂COOH |
| OCF₃ | H | H | CONHC(CH₂)₂CONH₂ |
| OCF₃ | H | H | CON(CH₂)₂N(CH₃)₂ |
| OCF₃ | H | H | CONHCH₃ |
| OCF₃ | H | H | CON(CH₃)₂ |
| OCF₃ | H | H | COOCH₃ |
| OCF₃ | H | H | CONHCH(CH₃)CONH₂(S) |
| OCF₃ | H | H | 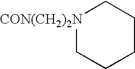 |
| OCF₃ | H | H | CONHC(CH₃)₃ |
| OCF₃ | H | H | CON(CH₃)₂CH₂OH |
| OCF₃ | H | H | CONHCH(CH₃)CONH₂(R) |
| OCF₃ | H | H | 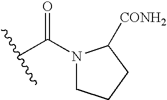 |
| OCF₃ | H | CH₃ | CH₃ |
| OCF₃ | H | CH₃ | COOH |
| OCF₃ | H | CH₃ | CONH₂ |
| OCF₃ | H | H | CONHCH₂CONH₂ |
| OCF₃ | H | Cl | CH₃ |
| OCF₃ | H | Cl | CONH₂ |
| OCF₃ | H | H | NHCONH₂ |
| CF₃ | H | H | CH₃ |
| CF₃ | H | H | H |
| CF₃ | H | H | COOH |
| CF₃ | H | H | CONH₂ |
| CF₃ | H | H | 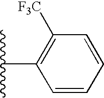 |
| CF₃ | H | H | SH |
| CF₃ | H | H | S—COCH₃ |
| CF₃ | H | H | Cl |
| CF₃ | H | H | CN |
| CF₃ | H | H | 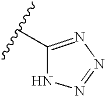 |
| CF₃ | 5-F | H | CH₃ |
| CF₃ | 5-F | H | COOH |
| CF₃ | 5-F | H | CONH₂ |
| CF₃ | 4-F | H | CONH₂ |
| CF₃ | 4-Cl | H | CONH₂ |
| Cl | 6-Cl | H | CONH₂ |
| CF₃ | 6-CF₃ | H | COOH |
| CF₃ | 6-CF₃ | H | CONH₂ |
| CF₃ | 4-CF₃ | H | CH₃ |
| CF₃ | 4-CF₃ | H | COOH |
| CF₃ | 4-CF₃ | H | CONH₂ |
| CF₃ | H | H | 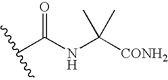 |
| O-Ph | H | H | CH₃ |
| O-Ph | H | H | COOH |
| O-Ph | H | H | CONH₂ |
| H | O-Ph | H | CONH₂ |
| Cl | H | H | CH₃ |
| H | 3-Cl | H | CH₃ |
| —SO₂NH—tBu | H | H | CH₃ |
| —SO₂NH₂ | H | H | CH₃ |
| —CONH—tBu | H | H | CH₃ |
| —CONH2. | H | H | CH₃ |
| —CONH—tBu | H | H | COOH |
| —CONH—tBu | H | H | CONH₂ |
| Cl | 3-Cl | H | COOH |
| Cl | 3-Cl | H | CONH₂ |
| Cl | 3-Cl | H | COOCH₃ |
| —SO₂NH—tBu | H | H | COOH |
| —SO₂NH₂ | H | H | COOH |
| —SO₂NH—tBu | H | H | CONH₂ |
| —SO₂NH₂ | H | H | CONH₂ |
| OtBu | H | H | CH₃ |
| OtBu | H | H | COOH |
| OtBu | H | H | CONH₂ |
| 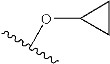 | H | H | CH₃ |
| 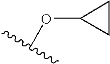 | H | H | COOH |
| 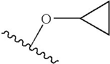 | H | H | CONH₂ |
| OCH₂CF₃ | H | H | CH₃ |
| OCH₂CF₃ | H | H | COOH |
| OCH₂CF₃ | H | H | CONH₂ |
| CHO | H | H | CONH₂ |
| H | 3-CF₃ | H | CONH₂ |
| H | 4-CF₃ | H | CONH₂ |
| H | 3-F | H | CONH₂ |
| H | 4-Cl | H | CONH₂ |
| H | 4-F | H | CONH₂ |
| 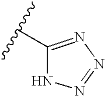 | H | H | CONH₂ |
| OCH₃ | 3-OCH₃ | H | CONH₂ |
| OCH₃ | 5-Cl | H | CONH₂ |
| CH₃ | H | H | CONH₂ |
| CH₃ | 3-F | H | CONH₂ |
| 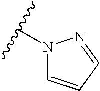 | H | H | CONH₂ |
| H | 4-(CH₂OH) | H | CONH₂ |
| H | 3-Cl | H | CONH₂ |
| H | 3-OEt | H | CONH₂ |
| H | 4-OEt | H | CONH₂ |
| F | H | H | CONH₂ |
| CH₃ | 6-CH₃ | H | CONH₂ |
| H | 4-tBu | H | CONH₂ |
| H | 4-OCF₃ | H | CONH₂ |
| H | 4-COCH₃ | H | CONH₂ |
| H | 3-COCH₃ | H | CONH₂ |
| H | 3-(CH₂OH) | H | CONH₂ |
| H | 4-GN | H | CONH₂ |
| H | 3-OCF₃ | H | CONH₂ |
| F | 4-F | H | CONH₂ |
| H | H | H | CONH₂ |
| OCF₃ | 4-N(Me)SO₂Me | H | CH₃ |
| OCF₃ | 4-N(Me)SO₂Me | H | CONH₂ |
| OCF₃ | 4-NHCO—tBu | H | CH₃ |
| OCF₃ | 4-NHCO—tBu | H | COOH |
| OCF₃ | 4-NHCO—tBu | H | CONH₂ |
| OCF₃ | H | H | 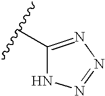 |
| OCF₃ | H | H | 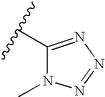 |
| OCF₃ | H | H | 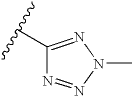 |
| OCF₃ | H | H | 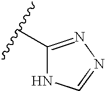 |
| OCF₃ | H | H | —CH₂CONH₂ |
| OCF₃ | H | H | —CH₂CN |
| OCF₃ | H | H | —SO₂NHtBu |
| OCF₃ | H | H | —SO₂NH₂ |
| OCF₃ | H | H | —SO₂NHMe |
| OCF₃ | H | H | —CH₂OH |
| OCF₃ | H | H | —CH(Me)OH |
| OCF₃ | H | H | —CH₂NHCOCH₃ |
| OCF₃ | H | H | —CH₂OSO₂NH₂ |
| OCF₃ | H | H | —NHCH₃ |
| OCF₃ | H | H | —NH—CH(CH₃)₂ |
| OCF₃ | H | H | 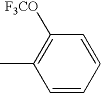 |

or a pharmaceutically acceptable salt thereof.

35. The compound of claim 1 represented by

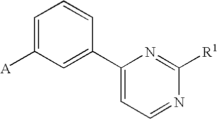

| A | R¹ |
|---|---|
| 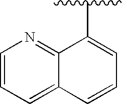 | CONH₂ |
| 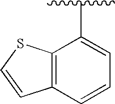 | CONH₂ |
| 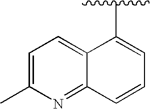 | CONH₂ |
| 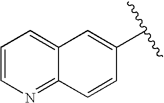 | CONH₂ |
| 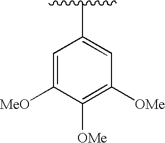 | CONH₂ |
| 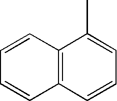 | CONH₂ |
| 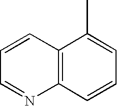 | CONH₂ |
| 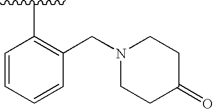 | CONH₂ |
| 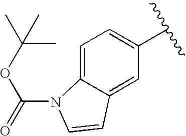 | CONH₂ |

or a pharmaceutically acceptable salt thereof.

36. The compound of claim 1 represented by

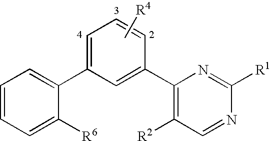

| R⁶ | R⁴ | R² | R¹ |
|---|---|---|---|
| OCF₃ | 4-F | H | CH₃ |
| OCF₃ | 4-F | H | COOH |
| OCF₃ | 4-F | H | COOCH₃ |
| OCF₃ | 4-F | H | CONH₂ |
| CF₃ | 4-F | H | COOCH₃ |
| CF₃ | 4-F | H | CONH₂ |
| CF₃ | 4-F | H | CH₃ |
| OCF₃ | 2-OCH₂Ph | H | CH₃ |
| OCF₃ | 2-OH | H | CH₃ |
| OCF₃ | 4-NHAc | H | CH₃ |
| OCF₃ | 4-NHAc | H | COOCH₃ |
| OCF₃ | 4-NHAc | H | CONH₂ |
| OCF₃ | 2-F | H | CH₃ |
| OCF₃ | 2-F | H | COOCH₃ |
| OCF₃ | 2-F | H | CONH₂ |
| OCF₃ | 4-Br | H | CH₃ |
| OCF₃ | 4-Br | H | COOCH₃ |
| OCF₃ | 4-Br | H | CONH₂ |
| OCF₃ | 4-Br | H | COOH |
| OCF₃ | 4-Ph | H | CH₃ |
| OCF₃ | 4-Ph | H | COOCH₃ |
| OCF₃ | 4-Ph | H | CONH₂ |
| OCF₃ | 4-Cl | H | CH₃ |
| OCF₃ | 4-Cl | H | COOCH₃ |
| OCF₃ | 4-Cl | H | COOH |
| OCF₃ | 4-Cl | H | CONH₂ |
| OCF₃ | 2-Cl | H | CH₃ |
| OCF₃ | 2-Cl | H | COOCH₃ |
| OCF₃ | 2-Cl | H | CONH₂ |
| OCH₂CF₃ | 4-F | H | CH₃ |
| OCH₂CF₃ | 4-F | H | COOCH₃ |
| OCH₂CF₃ | 4-F | H | COOH |
| OCH₂CF₃ | 4-F | H | CONH₂ |
| H | 4-OCH₂CF₃ | H | CONH₂ |
| OCF₃ | 4-F | CH₃ | CH₃ |
| OCF₃ | 4-F | CH₃ | COOCH₃ |
| OCF₃ | 4-F | CH₃ | CONH₂ |
| F | 4-OCH₂CF₃ | H | CONH₂ |

or a pharmaceutically acceptable salt thereof.

37. The compound of claim 1 represented by

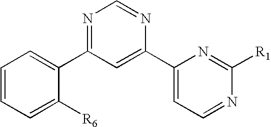

| R⁶ | R² |
|---|---|
| CF₃ | CH₃ |
| CF₃ | COOH |
| CF₃ | CONH₂ |
| OCF₃ | CH₃ |
| OCF₃ | COOH |
| OCF₃ | CONH₂ |

or a pharmaceutically acceptable salt thereof.

38. A compound represented by 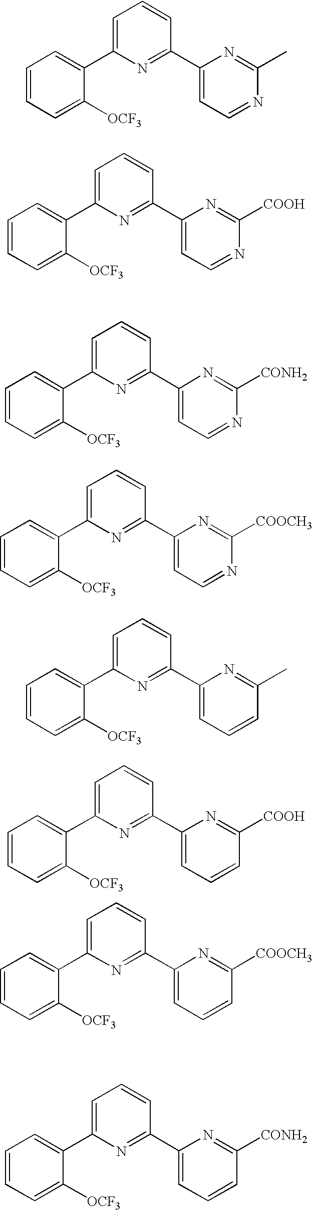
or a pharmaceutically acceptable salt thereof.

39. The compound of claim 1 represented by

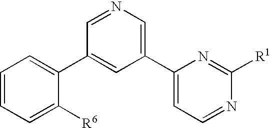

| R⁶ | R² |
|---|---|
| OCF₃ | CH₃ |
| OCF₃ | COOH |
| OCF₃ | COOCH₃ |
| OCF₃ | CONH₂ |

or a pharmaceutically acceptable salt thereof.

40. The compound of claim 1 represented by

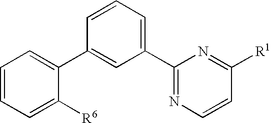

| R⁶ | R² |
|---|---|
| OCF₃ | COOH |
| OCF₃ | CONH₂ |
| CF₃ | CH₃ |
| CF₃ | COOH |
| CF₃ | CONH₂ |

or a pharmaceutically acceptable salt thereof.

41. The compound of claim 1 represented by

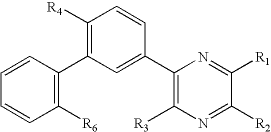

| R⁶ | R⁴ | R³ | R² | R¹ |
|---|---|---|---|---|
| OCF₃ | H | H | H | CH₃ |
| OCF₃ | H | H | H | COOH |
| OCF₃ | H | H | H | CONH₂ |
| OCF₃ | H | H | H | COOCH₃ |
| CF₃ | H | H | H | COOH |
| CF₃ | H | H | H | CONH₂ |
| Cl | H | H | H | CONH₂ |
| OCF₃ | H | H | H | CONHC(CH₃)₂CONH₂ |
| OCF₃ | H | H | H | COCH₃ |
| OCF₃ | H | H | H | CH(OH)CH₃ |
| OCF₃ | H | H | H | COCF₃ |
| OCF₃ | H | H | H | CH(OH)CF₃ |
| OCF₃ | H | H | H | SOCH₃ |
| OCF₃ | H | H | H | SO₂CH₃ |
| OCF₃ | H | H | H | NHSO₂CH₃ |
| OCF₃ | H | H | CH₃ | NHSO₂CH₃ |
| OCF₃ | H | H | H | NHCO₂CH₃ |
| OCF₃ | H | H | H | NHCOCH₃ |
| OCF₃ | H | H | H | NHCONH₂ |
| OCF₃ | H | H | H | NHSO₂NH₂ |
| OCF₃ | H | H | H | N(CH₃)CONH₂ |
| OCF₃ | H | H | CH₃ | N(CH₃)CONH₂ |
| OCF₃ | H | H | N(CH₃)CONH₂ | CH₃ |
| OCF₃ | H | H | H | 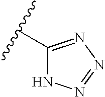 |
| OCF₃ | H | H | H | 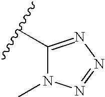 |
| OCF₃ | H | H | H | 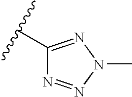 |
| OCF₃ | H | H | H | 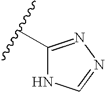 |
| OCF₃ | H | H | H | 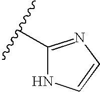 |
| OCF₃ | H | H | H | 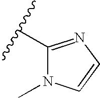 |
| OCF₃ | H | H | H | —CH₂CH₂CONH₂ |
| OCF₃ | H | H | H | —CH₂CONH₂ |
| OCF₃ | H | H | H | —CH₂CN |
| OCF₃ | H | H | H | —SO₂NH-tBu |
| OCF₃ | H | H | H | —SO₂NH₂ |
| OCF₃ | H | H | H | —SO₂NHMe |
| OCF₃ | H | H | H | —CH₂OH |
| OCF₃ | H | H | H | —CH(Me)OH |
| OCF₃ | H | H | H | —CH₂NHCOCH₃ |
| OCF₃ | H | H | H | —CH₂OSO₂NH₂ |
| OCF₃ | H | H | H | —NHCH₃ |
| OCF₃ | H | H | H | —NH—CH(CH₃)₂ |
| OCF₃ | H | H | H | NH₂ |
| OCF₃ | H | H | CH₃ | OCH₃ |
| OCF₃ | H | H | OCH₃ | CH₃ |
| OCF₃ | H | H | CH₃ | OH |
| OCF₃ | H | H | OH | CH₃ |
| OCF₃ | H | NH₂ | NH₂ | CONH₂ |
| OCF₃ | F | H | H | CONH₂ |
| OCF₃ | H | H | CH₃ | OCON(CH₃)₂ |
| OCF₃ | H | H | OCON(CH₃)₂ | CH₃ |
| OCF₃ | H | H | CONH₂ | OCH₃ |
| OCF₃ | H | H | CH₃ | O(CH₂)₂N(CH₃)₂ |
| OCF₃ | H | H | O(CH₂)₂N(CH₃)₂ | CH₃ |
| OCF₃ | H | H | OCH₃ | CONH₂ |
| OCF₃ | H | H | CH₃ | NHCH₃ |
| OCF₃ | H | H | Cl | CH₃ |
| OCF₃ | H | H | CH₃ | H |
| OCF₃ | H | H | H | CH₃ |
| OCF₃ | H | H | CONH₂ | H |
| OCF₃ | F | H | CONH₂ | H |
| OCF₃ | H | H | H | SCH₃ |
| OCF₃ | H | H | H | S(O)CH₃ |
| OCF₃ | H | H | H | SO₂CH₃ |
| OCF₃ | F | H | H | COOH |
| OCF₃ | H | H | H | CHO |
| OCF₃ | H | H | H | COCH₃ |
| OCF₃ | H | H | H | CN |
| OCF₃ | H | H | H | H |
| OCF₃ | H | H | H | 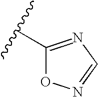 |
| OCF₃ | H | H | H | CH(OH)CF₃ |
| OCF₃ | H | H | CH(OH)CF₃ | H |
| OCF₃ | H | H | CONH₂ | OH |
| OCF₃ | H | H | CH₃ | CONH-tBu |
| OCF₃ | H | H | H | COCF₃ |
| OCF₃ | H | H | H | —OCH₂SO₂NH₂ |
| OCF₃ | H | H | H | —CH═CHCO₂CH₃ |
| OCF₃ | H | H | H | —CH(NH₂)CH₂CONH₂ |
| OCF₃ | H | H | CONH₂ | OCH₃ |
| OCF₃ | H | H | H | —CONHCH(CH₃)CONH₂ |
| OCF₃ | H | H | H | —CON(CH₃)₂ |
| OCF₃ | H | H | H | —O(CH₂)₂N(CH₃)₂ |
| OCF₃ | H | H | H | —CH₂NHCOCH₃ |
| CF₃ | H | H | H | COOCH₃ |
| OCF₃ | H | H | H | S—COCH₃ |
| CF₃ | H | H | H | CONH₂ |
| OPh | H | H | H | CONH₂ |
| OCF₃ | H | H | H | CONHCH₃ |
| OCF₃ | H | H | NH₂ | NHCH₃ |
| OCF₃ | H | H | NH₂ | COOPr |
| Cl | H | H | H | COOCH₃ |
| OCF₃ | H | H | NH₂ | CONH₂ |
| Cl | H | H | H | CONH₂ |
| OCF₃ | H | H | H | CSNH₂ |
| OCF₃ | H | H | CH₃ | CONH₂ |
| OCF₃ | H | H | OCH₃ | CONH₂ |
| OCF₃ | H | H | H | NHCOCH₃ |
| OCF₃ | H | H | H | N(COCH₃)₂ |
| OCF₃ | H | H | CH₃ | COOH |
| OCF₃ | H | H | CONH₂ | CONH₂ |
| OCF₃ | H | H | CH(CH₃)₂ | CONH₂ |
| OCF₃ | H | H | CONH₂ | CH(CH₃)₂ |
| OCF₃ | H | H | CH(CH₃)₂ | CONHC(═NH)NH₂ |
| OCF₃ | H | H | CH(CH₃)₂ | CONHOH |
| OCF₃ | H | H | H | NHCONH₂ |
| OCF₃ | H | CH₃ | H | CONH₂ |
| OCF₃ | H | CH₃ | CONH₂ | H |
| OCF₃ | H | H | H | NHCH₂CONH₂ |
| OCF₃ | H | H | H | NHC(═NH)NH₂ |
| OCF₃ | H | H | H | C(═NH)NH₂ |
| CF₃ | H | H | H | COOH |
| OCF₃ | H | Cl | H | CONH₂ |
| OCF₃ | H | CH₃ | COOH | H |
| OCF₃ | H | CH₃ | H | COOH |
| OCF₃ | H | NH₂ | H | CONH₂ |
| OCF₃ | H | NH₂ | H | COOH |
| OCF₃ | H | Cl | H | COOH |
| OCF₃ | H | NH₂ | CONH₂ | H |
| OCF₃ | H | CONH₂ | H | CONH₂ |
| OCH₂CF₃ | H | H | H | CONH₂ |
| OCH₂CF₃ | H | H | CONH₂ | H |
| OCH₂CF₃ | H | H | H | H |
| OCH₂CF₃ | H | H | H | COOH |
| 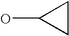 | H | H | H | COOCH₃ |
| 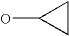 | H | H | H | CONH₂ |
| OCF₃ | H | H | H | CONHC(CH₃)₂CONH₂ |
| OCF₃ | H | H | H | CH(OH)CH₃ |
| OCF₃ | H | H | H | NHSO₂NH₂ |
| OCF₃ | H | H | H | N(CH₃)CONH₂ |
| OCF₃ | H | H | CH₃ | N(CH₃)CONH₂ |
| OCF₃ | H | H | N(CH₃)CONH₂ | CH₃ |

or a pharmaceutically acceptable salt thereof.

42. The compound of claim 1 represented by

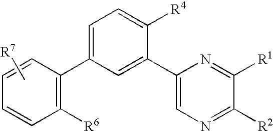

| R⁶ | R⁷ | R⁴ | R² | R¹ |
|---|---|---|---|---|
| CF₃ | 5-F | H | H | CONH₂ |
| CF₃ | 5-F | H | CONH₂ | H |
| CF₃ | 4-CF₃ | H | H | CONH₂ |
| OCF₃ | H | F | H | CONH₂ |
| OCF₃ | H | F | CONH₂ | H |
| CF₃ | 4-CF₃ | H | CONH₂ | H |
| CF₃ | 4-CF₃ | H | H | H |
| Cl | 3-Cl | H | H | COOCH₃ |
| Cl | 4-Cl | H | H | COOCH₃ |
| Cl | 3-Cl | H | H | CONH₂ |
| Cl | 4-Cl | H | H | CONH₂ |
| Cl | 6-Cl | H | H | CONH₂ |

or a pharmaceutically acceptable salt thereof.

43. A compound represented by 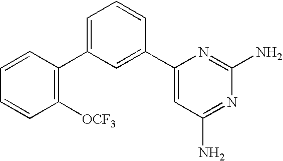
or a pharmaceutically acceptable salt thereof.

44. A pharmaceutical composition comprising a therapeutically effective amount of the compound according to claim 1, or a pharmaceutically acceptable salt thereof; and a pharmaceutically acceptable carrier.

45. The pharmaceutical composition according to claim 42, further comprising a second therapeutic agent selected from the group consisting of: i) opiate agonists, ii) opiate antagonists, iii) calcium channel antagonists, iv) 5HT receptor agonists, v) 5HT receptor antagonists vi) sodium channel antagonists, vii) NMDA receptor agonists, viii) NMDA receptor antagonists, ix) COX-2 selective inhibitors, x) NK1 antagonists, xi) non-steroidal anti-inflammatory drugs, xii) selective serotonin reuptake inhibitors, xiii) selective serotonin and norepinephrine reuptake inhibitors, xiv) tricyclic antidepressant drugs, xv) norepinephrine modulators, xvi) lithium, xvii) valproate, and xviii) neurontin.

46. A method of treatment or prevention of pain comprising the step of administering to a patient in need thereof a therapeutically effective amount, or a prophylactically effective amount, of the compound according to claim 1, or a pharmaceutically acceptable salt thereof.

47. A method of treatment of chronic, visceral, inflammatory or neuropathic pain syndromes comprising the step of administering to a patient in need thereof a therapeutically effective amount, or a prophylactically effective amount, of the compound according to claim 1, or a pharmaceutically acceptable salt thereof.

48. A method of treatment of pain resulting from, or associated with, traumatic nerve injury, nerve compression or entrapment, postherpetic neuralgia, trigeminal neuralgia, diabetic neuropathy, cancer or chemotherapy, comprising the step of administering to a patient in need thereof a therapeutically effective amount, or a prophylactically effective amount, of the compound according to claim 1, or a pharmaceutically acceptable salt thereof.

49. A method of treatment of chronic lower back pain comprising the step of administering to a patient in need thereof a therapeutically effective amount, or a prophylactically effective amount, of the compound according to claim 1, or a pharmaceutically acceptable salt thereof.

50. A method of treatment of phantom limb pain comprising the step of administering to a patient in need thereof a therapeutically effective amount, or a prophylactically effective amount, of the compound according to claim 1, or a pharmaceutically acceptable salt thereof.

51. A method of treatment of HIV- and HIV treatment-induced neuropathy, chronic pelvic pain, neuroma pain, complex regional pain syndrome, chronic arthritic pain or related neuralgias comprising the step of administering to a patient in need thereof a therapeutically effective amount, or a prophylactically effective amount, of the compound according to claim 1, or a pharmaceutically acceptable salt thereof.

52. A method of administering local anesthesia comprising the step of administering to a patient in need thereof a therapeutically effective amount, or a prophylactically effective amount, of the compound according to claim 1, or a pharmaceutically acceptable salt thereof.

53. A method of treatment of irritable bowel syndrome or Crohns disease comprising the step of administering to a patient in need thereof a therapeutically effective amount, or a prophylactically effective amount, of the compound according to claim 1, or a pharmaceutically acceptable salt thereof.

54. A method of treatment of epilepsy or partial and generalized tonic seizures comprising the step of administering to a patient in need thereof a therapeutically effective amount, or a prophylactically effective amount, of the compound according to claim 1, or a pharmaceutically acceptable salt thereof.

55. A method for neuroprotection under ischaemic conditions caused by stroke or neural trauma comprising the step of administering to a patient in need thereof a therapeutically effective amount, or a prophylactically effective amount, of the compound according to claim 1, or a pharmaceutically acceptable salt thereof.

56. A method of treatment of multiple sclerosis comprising the step of administering to a patient in need thereof a therapeutically effective amount, or a prophylactically effective amount, of the compound according to claim 1, or a pharmaceutically acceptable salt thereof.

57. A method of treatment of bipolar disorder comprising the step of administering to a patient in need thereof a therapeutically effective amount, or a prophylactically effective amount, of the compound according to claim 1, or a pharmaceutically acceptable salt thereof.

58. A method of treatment of tachy-arrhythmias comprising the step of administering to a patient in need thereof a therapeutically effective amount, or a prophylactically effective amount, of the compound according to claim 1, or a pharmaceutically acceptable salt thereof.
